(19) United States
(12) Patent Application Publication
(10) Pub. No.: US 2014/0228543 A1
(43) Pub. Date: Aug. 14, 2014

(54) MODIFIED AND DERIVATIZED ß-GLUCAN COMPOUNDS, COMPOSITIONS, AND METHODS

(71) Applicant: Biothera, Inc., Eagan, MN (US)

(72) Inventors: Nandita Bose, Plymouth, MN (US); William J. Grossman, Third Lake, IL (US); Michael Danielson, St. Paul, MN (US); Andrew Magee, Maynard, MA (US); Paul Will, Marlborough, MA (US); Kyle S. Michel, Eagan, MN (US); Vanessa A. Iiams, Bloomington, MN (US); Xiaohong Qiu, Edina, MN (US); Mary Antonysamy, Woodbury, MN (US)

(21) Appl. No.: 14/115,824

(22) PCT Filed: May 7, 2012
(86) PCT No.: PCT/US2012/036795

§ 371 Date: Apr. 28, 2014

Related U.S. Application Data
(60) Provisional application No. 61/483,353, filed on May 6, 2011.

(51) Int. Cl. C07K 17/10

(52) U.S. Cl. CPC C07K 17/10
USPC 530/363; 530/387.3

(57) ABSTRACT

Modified and/or derivativized β-glucans with an enhanced capacity to modulate human immune response as compared to the parent, unmodified and/or underivatized β-glucans are demonstrated.

### BACKGROUND

Pathogen-associated molecular patterns (PAMPs) are molecules unique to pathogens that are recognized by innate immune cells. Recognition of these PAMPs by the innate immune cells through evolutionarily ancient, germline-encoded pattern recognition receptors (PRRs) enables them to orchestrate a non-specific immune response against the pathogen. PRRs can be broadly classified based on their location, a) serum or tissue fluid and b) membrane or cytoplasmic. Complement proteins in the serum, specifically C3 and C1q, are examples of serum PRR that recognize pathogens. Toll-like receptors (TLRs) are one of the classes of PRRs present on the cell membrane or in the cytoplasm of leukocytes.

Yeast-derived β-glucans are fungal PAMPs that have been extensively evaluated for their immunomodulatory properties. Structurally, yeast β-glucans are composed of glucose monomers organized as a β-(1-3)-linked glucopyranose backbone with periodic β-(1-3) glucopyranose branches linked to the backbone via β-(1-6)glycosidic linkages. The mechanism(s) through which the yeast β-glucans exert their immunomodulatory effects has largely been influenced by the most basic and simple structural difference, such as its particulate or soluble forms. Particulate glucans were shown to induce a number of innate and adaptive immune functions including phagocytosis, oxidative burst, induction of a variety of cytokines and chemokines, and inhibition of tumor growth (5, 6). Soluble glucans have been extensively evaluated for their anti-tumor activities. Low molecular weight soluble glucans (<40 kDa) were shown to prime murine or human NK cells, neutrophils and macrophages for cytotoxicity against tumor cells (7-12). Medium molecular weight glucans (˜120-205 kDa) that include Imprime PGG®, in combination with tumor-specific monoclonal antibodies (MAbs), were shown to inhibit tumor growth and enhance long-term survival over MAbs or β-glucan alone in multiple tumor models (10, 13, 14). Other immunobiological activities of β-glucans that have been reported include, enhancing wound healing and eliciting anti-inflammatory responses (5).

### SUMMARY OF THE INVENTION

Modified and/or derivativized β-glucans with an enhanced capacity to modulate human immune response as compared to the parent, unmodified and/or underivatized β-glucans are demonstrated. The modified and derivativized β-glucans show increased ability to be recognized by PRRs by demonstrating, a) enhanced activation of complement and induction of greater opsonization as measured by increased staining of iC3b on the β-glucan-bound cell, and b) enhanced binding to human neutrophils as detected by increased staining of BfD IV, the monoclonal antibody specific to β-1,3/1,6 glucans and c) enhanced activation of immune functions, such as oxidative burst measured by spectrophotometric assay of cytochrome c reduction by reactive oxygen intermediates.

### BRIEF DESCRIPTION OF THE FIGURES

FIG. 1 shows various chemical approaches for modified and/or derivatized β-glucan synthesis.

FIG. 2 shows migration of a β-glucan analog.

FIG. 3 shows detection of iC3b on human neutrophils with the parent and derivatized MMW yeast β-glucan.

FIG. 4 shows the comparison of induction of oxidative burst in human peripheral blood mononuclear cells by parent and derivatized MMW yeast β-glucans.

FIG. 5 shows the comparison of induction of oxidative burst in human PBMCs by scleroglucan, modified scleroglucan, and MMW yeast β-glucan.

### DETAILED DESCRIPTION OF ILLUSTRATIVE EMBODIMENTS

Examples of advantages of modified and/or derivatives of β-glucans include enhanced ability to activate complement pathways and become opsonized, enhanced recognition by a repertoire of immune cells expressing complement receptors (eg, B cells, neutrophils, and macrophages) and enhancement of the known β-glucan-induced immune functions, such as phagocytosis, oxidative burst, modulation of immune receptors/pathways, cytokines and chemokines expression, reduction of tumor cell burden, increased wound healing and increased infectious organism clearance.

Enhancement of modulation of immune function in disease states where β-glucans can be used in combination with other therapeutic agents, such as: cancer chemotherapy or monoclonal antibodies, tumor vaccines, antibiotics, and infectious disease vaccines.

Different forms of β-glucan exist. A β-glucan polysaccharide can exist in at least four distinct conformations: single disordered chains, single helix, single triple helix, and triple helix aggregates. As used herein, the term “single triple helix” refers to a β-glucan conformation in which three single chains are joined together to form a triple helix structure. In a single triple helix conformation, there is no higher ordering of the triple helices—i.e., there is substantially no aggregation of triple helices. As used herein, the term “triple helix aggregate” refers to a β-glucan conformation in which two or more triple helices are joined together via non-covalent interactions. A β-glucan composition can include one or more of these forms, depending upon such conditions as pH and temperature. Saccharomyces contain β-1,3/1,6 glucan, which is the glucan form of poly-(1-6)-β-D-glucopyranosyl-(1-3)-β-D-glucopyranose (PGG, Biothera, Eagan, Minn.), making it an attractive antigenic target for fungal vaccine development. The β-glucan moiety can alternatively be provided in the form of neutral soluble glucan. As used herein, “neutral soluble β-glucan” refers to an aqueous soluble β-glucan having a unique triple helical conformation that results from the denaturation and re-annealing of aqueous soluble glucan.

In some embodiments, the β-glucan moiety can include PGG, containing β(1-3) and β(1-6) linkages in varying ratios depending on the organism from which it is obtained and the processing conditions employed. PGG glucan preparations can contain neutral glucans, which have not been modified by substitution with functional (e.g., charged) groups or other covalent attachments. The biological activity of PGG glucan can be controlled by varying the average molecular weight and the ratio of β(1-6) to β(1-3) linkages of the glucan molecules. The average molecular weight of soluble glucans generally can be from about 10,000 daltons to about 500,000 daltons. In some embodiments, the average molecular weight of soluble glucans can be from about 30,000 daltons to about 50,000 daltons.

More generally, the β-glucan moiety can include a polymer of glucose monomers organized as a β(1-3) linked glucopyranose backbone with periodic branching via β(1-6)glycosidic linkages. In some embodiments, the β-glucan is substantially unsubstituted with functional (e.g., charged) groups or other covalent attachments. One form of the β-glucan is produced by dissociating the native glucan conformations and then re-annealing and purifying the resulting triple helical conformation. The triple helical conformation of the β-glucan moiety contributes to the β-glucan's ability to selectively activate the immune system without stimulating the production of detrimental biochemical mediators.

Soluble forms of β-glucans can be prepared from insoluble glucan particles such as, for example, insoluble glucan particles derived from yeast organisms as described herein. However, in some embodiments, a particulate form of β-glucan may be used. Other strains of yeast from which insoluble glucan particles can be obtained include, for example, Saccharomyces delbrueckii, Saccharomyces rosei, Saccharomyces microellipsodes, Saccharomyces carlsbergensis, Schizosaccharomyces pombe, Kluyveromyces lactis, Kluyveromyces fragilis, Kluyveromyces polysporus, Candida albicans, Candida cloacae, Candida tropicalis, Candida utilis, Hansenula wingeri, Hansenula arni, Hansenula henricii, Hansenula americana.

As described above, certain conformations of β-glucan can include aggregates of single chains such as, for example, a single triple helix (an aggregate of three single helices) or a triple helix aggregate (an aggregate of triple helices). The “aggregate number” of a β-glucan conformation is the number of single chains which are joined together in that conformation. For example, the aggregate number of a single helix is 1, the aggregate number of a single triple helix is 3, and the aggregate number of a triple helix aggregate is greater than 3. For example, a triple helix aggregate consisting of two triple helices joined together has an aggregate number of 6.

The aggregate number of a β-glucan sample under a specified set of conditions can be determined by determining the average molecular weight of the polymer under those conditions. The β-glucan is then denatured, that is, subjected to conditions which separate any aggregates into their component single polymer chains. The average molecular weight of the denatured polymer is then determined. The ratio of the molecular weights of the aggregated and denatured forms of the polymer is the aggregate number. A typical β-glucan composition includes molecules having a range of chain lengths, conformations and molecular weights. Thus, the measured aggregate number of a β-glucan composition is the mass average aggregate number across the entire range of β-glucan molecules within the composition. It is to be understood that any reference herein to the aggregate number of a β-glucan composition refers to the mass average aggregate number of the composition under the specified conditions. The aggregate number of a composition indicates which conformation is predominant within the composition. For example, a measured aggregate number of about 6 or more is characteristic of a composition in which the β-glucan is substantially in the triple helix aggregate conformation.

The conformation of a PGG-glucan preparation can be temperature dependent; PGG-glucan can be predominantly in a triple helix aggregate conformation at 25° C., but can be a mixture of triple helix aggregates and the single triple helix conformation 37° C.

In certain embodiments, soluble β-glucan can be substantially in a triple helix aggregate conformation under physiological conditions—e.g., physiological pH, about pH 7, and physiological temperature, about 37° C. In one particular embodiment, the β-glucan consists essentially of β-glucan chains in one or more triple helix aggregate conformations under physiological conditions.

In one embodiment, soluble β-glucan composition can be characterized by an aggregate number under physiological conditions of greater than about 6 such as, for example, an aggregate number of the β-glucan composition under physiological conditions of at least about 7, at least about 8, or at least about 9.

In some embodiments, the β-glucan moiety can posses a specified molecular weight. As used herein, the “molecular weight” of a β-glucan moiety can refer to either the weight average molecular weight (Mw) or the number average molecular weight (Mn). These values may be determined using size exclusion chromatography (SEC) with universal calibration or multi angle light scattering (MALS) detection. The conditions under which molecular weight is measured such as, for example, temperature and the type of elution buffer can influence the aggregation state of the β-glucan and thus also influence the reported value for molecular weight. Unless otherwise specifically indicated, β-glucan molecular weight values are reported as weight average molecular weight values obtained via SEC in a pH=7 buffer at 18° C. with MALS detection.

Thus, the β-glucan moiety can have a weight average molecular weight of, for example, no greater than 4,000,000 daltons, no greater than 3,000,000 daltons, no greater than 2,000,000 daltons, no greater than 1,000,000 daltons, no greater than 500,000 daltons, no greater than 250,000 daltons, no greater than 200,000 daltons, no greater than 150,000 daltons, no greater than 100,000 daltons, no greater than 95,000 daltons, no greater than 90,000 daltons, no greater than 85,000 daltons, no greater than 80,000 daltons, no greater than 75,000 daltons, no greater than 70,000 daltons, no greater than 65,000 daltons, no greater than 60,000 daltons, no greater than 55,000 daltons, no greater than 50,000 daltons, no greater than 45,000 daltons, no greater than 40,000 daltons, no greater than 35,000 daltons, no greater than 30,000 daltons, no greater than 25,000 daltons, no greater than 20,000 daltons, or no greater than 15,000 daltons. The β-glucan moiety can have a molecular weight of at least 500 daltons, at least 1,000 daltons, at least 5,000 daltons, 10,000 daltons, at least 15,000 daltons, at least 20,000 daltons, at least 25,000 daltons, at least 30,000 daltons, at least 35,000 daltons, at least 40,000 daltons, at least 45,000 daltons, at least 50,000 daltons, at least 55,000 daltons, at least 60,000 daltons, at least 65,000 daltons, at least 70,000 daltons, or at least 75,000 daltons. Moreover, the β-glucan moiety can have a molecular weight within a range defined by any of the minimum molecular weights recited herein paired with any of the recited maximum molecular weights recited herein. Thus, for example, the β-glucan moiety can have a molecular weight of, for example, at least 1,000 daltons and no greater than 50,000 daltons, at least 5,000 daltons and no greater than 100,000 daltons, at least 10,000 daltons and no greater than 500,000, at least 25,000 daltons and no greater than 1,000,000 daltons, or at least 50,000 daltons and no greater than 4,000,000 daltons. In some embodiments the β-glucan moiety can have a weight average molecular weight of 150,000 daltons. In some embodiments the β-glucan moiety can have a weight average molecular weight of 390,000 daltons.

In some embodiments the β-glucan moiety may be derived from a yeast such as, for example, S. cerevisiae, in both soluble and particulate form. The β-glucan is a polymer of glucose, which is mostly in the β-1,3 linkage, but also contains one or more side chains linked to the backbone β-1,3 glucose polymer in a β-1,6 linkage. As used herein, a β-1,6 linkage refers to the linkage of the side chain to the β-1,3 glucose polymer regardless of the manner in which units of the side chain are linked to one another. In some embodiments, the β-glucan moiety can include one or more β-1,6-linked side chains that include two or more saccharide units.

In some embodiments, the side chain saccharide units can include, for example, a pyranose such as, for example, glucose, mannose, galactose, or fructose; a furanose such as, for example, ribose; or any combination thereof. In some embodiments, the β-1,6-linked side chain can includes at least two saccharide units, at least three saccharide units, at least four saccharide units, at least five saccharide units, at least six saccharide units, at least seven saccharide units, or at least eight saccharide units. In some embodiments, the β-1,6-linked side chain can include no more than two saccharide units, no more than three saccharide units, no more than four saccharide units, no more than five saccharide units, no more than six saccharide units, no more than seven saccharide units, or no more than eight saccharide units. Moreover, the β-1,6-linked side chain can include a number of saccharide units within a range defined by any of the minimum number of saccharide units recited herein paired with any of the recited maximum number of saccharide units recited herein.

Yeast β-glucans can be produced as an insoluble particle (whole glucan particle; WGP) or as a soluble form. WGP is a highly purified, β-1,3/1,6 glucan particle from Saccharomyces cell walls following a proprietary extraction process. Soluble glucan (β-(1,6)-[poly-1,3)-D-glucopyranosyl]-poly-b(1,3)-D-glucopyranose) is a highly purified glucose polymer prepared by acid hydrolysis of WGP β-glucan. Other soluble glucan fractions of varying molecular weight, aggregation state, and branching are possible as well. Thus, in some embodiments, the β-glucan moiety may be, or be derived from, that include, for example PGG (poly-(1-6)-β-D-glucopyranosyl-(1-3)-β-D-glucopyranose), soluble β-glucan (including, e.g., neutral soluble β-glucan), triple helical β-glucan (BETAFECTIN, Biothera, Eagan, Minn.) or β-glucans of various aggregate numbers. The above-mentioned species of β-glucans may be administered separately or in various combinations. The β-glucan moiety may be prepared from insoluble glucan particles.

The β-glucan may be formed from starting material that includes glucan particles such as, for example, whole glucan particles described by U.S. Pat. No. 4,810,646, U.S. Pat. No. 4,992,540, U.S. Pat. No. 5,082,936 and/or U.S. Pat. No. 5,028,703. The source of the whole glucan particles can be the broad spectrum of glucan-containing fungal organisms that contain β-glucans in their cell walls. Suitable sources for the whole glucan particles include, for example, Saccharomyces cerevisiae R4 (deposit made in connection with U.S. Pat. No. 4,810,646; (Agricultural Research Service No. NRRL Y-15903) and R4 Ad (American Type Culture Collection, Manassas, Va., ATCC No. 74181). The structurally modified glucans hereinafter referred to as “modified glucans” derived from S. cerevisiae R4 can be potent immune system activators (U.S. Pat. No. 5,504,079).

The whole glucan particles from which the β-glucan may be prepared can be in the form of a dried powder. In order to prepare the β-glucan, however, it is not necessary to perform the final organic extraction and wash steps described in one or more of these patent documents.

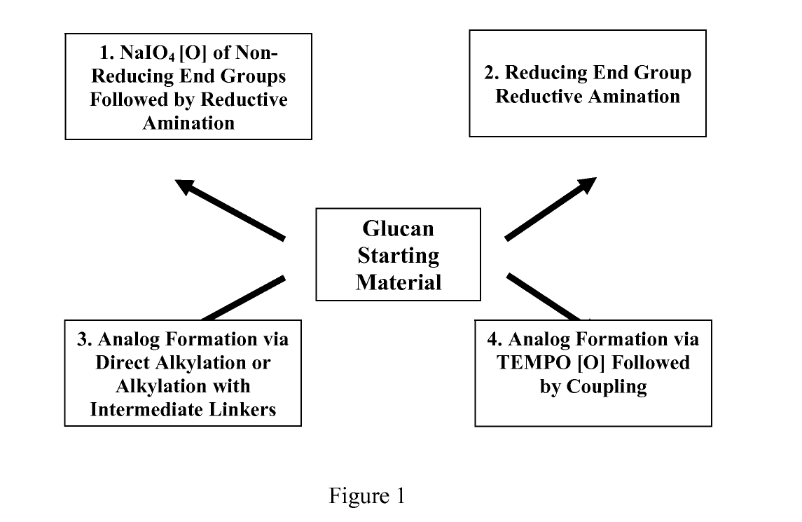

Several approaches for synthesizing β-glucan analogs are broadly summarized in FIG. 1. These approaches are discussed in further detail in the following sections. The starting glucan can be, for example, glucan derived from fungal yeast sources, for example, Saccharomyces cerevisiae, Torula (candida utilis), Candida albicans, and Pichia stipitis, or any other yeast source; glucan derived from other other fungal sources, for example, scleroglucan from Sclerotium rofsii or any other non-yeast fungal sources; glucan from algal sources, for example, laminarin or phycarine from Laminaria digitata or any other algal source; glucan from bacterial sources, for example, curdlan from Alcaligenes faecalis or any other bacterial source; glucan from mushroom sources, for example, schizophyllan from Schizophyllan commune, lentinan from Lentinan edodes, grifolan from Grifola frondosa, ganoderan from Ganoderma lucidum, krestin from Coriolus versicolor, pachyman from Poria cocos Wolf, or any other mushroom source; glucan derived from cereal grain sources, for example, oat glucan, barley glucan, or any other cereal grain source; glucan derived from lichen sources, for example, pustulan, from Umbilicaris pustulata, lichenan from Cetraria islandica, or any other lichenan source. The form of glucan used to make these conjugates can be either soluble or insoluble in water.

The first approach utilizes a periodate oxidation of the non-reducing termini residues of the β-glucan main- and side-chains to introduce reactive aldehyde moieties that will undergo a reductive amination reaction with an amine-containing molecule, for example benzyl amine.

Below shows the NaIO₄ oxidation of non-reducing terminal residues followed by coupling with benzyl amine, one example of an amine-containing molecule.

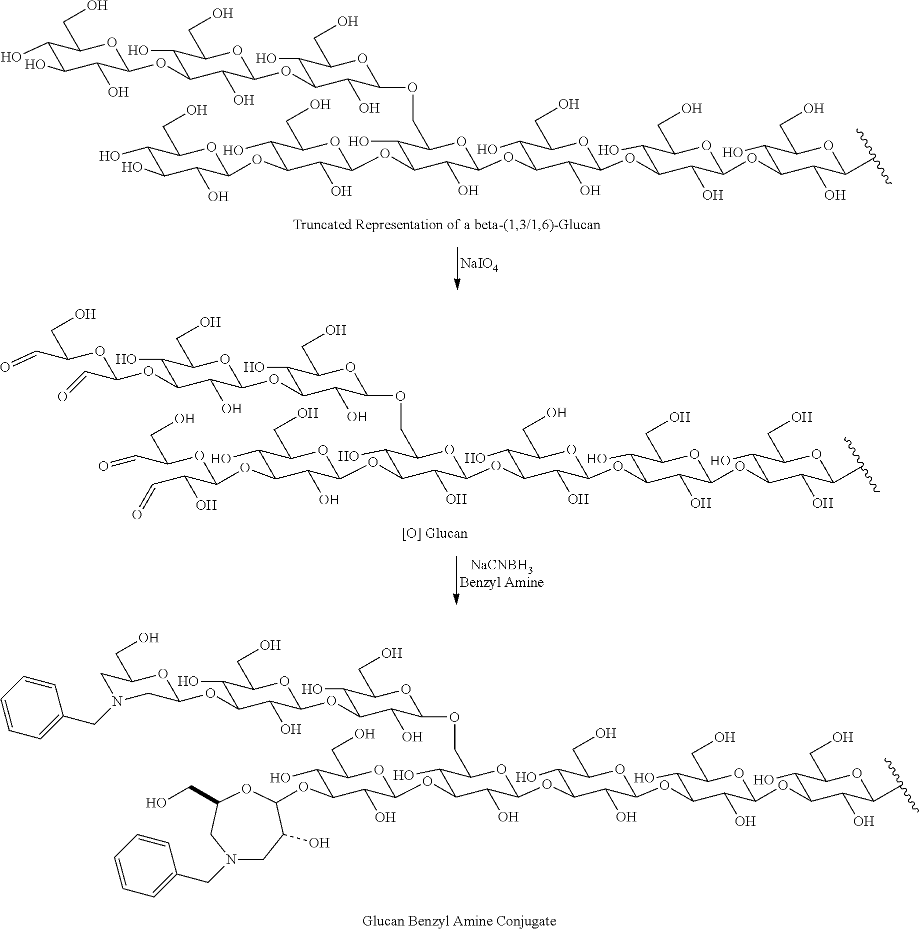

The first step of this approach is a NaIO₄ mediated oxidation of vicinal diols, which when dealing with 1,3/1,6 β-glucan, only exist on the single reducing termini residue and the non-reducing termini residues of the β-glucan main- and side-chains. This translates to a situation where the precise location of each newly formed di-aldehyde is known. Also, by varying the amount of oxidant, one can control the extent of the oxidation, thereby controlling the amount of amine-containing compound that is eventually incorporated. There are three types of di-aldehydes that can form on the non-reducing glucose residues based on the fact that a triol is present. If only one equivalent of sodium periodate cleaves one of the two possible diols then the resulting di-aldehyde can either be on the −2 and −3 ring carbons (2,3) or on the −3 and −4 ring carbons (3,4). Each of these di-aldehydes will lead to a different 7-membered ring [1,4]-oxazepane. If a second equivalent of sodium periodate further cleaves either the (2,3) or (3,4)di-aldehyde then the same product will be formed in both cases and the di-aldehydes will be on carbon −2 and −4 (2,4). This situation leads to a six membered ring morpholine derivative. The NaIO₄ treatment can be performed by treating an aqueous solution of β-glucan with NaIO₄ in the dark for 18-48 hours. The resulting solution is either quenched with ethylene glycol and dialyzed against water or carried into the subsequent step without further purification.

With the di-aldehyde functionality established, an amine-containing molecule may be attached by one of at least three approaches. The approach shown above involves a direct reductive amination between the di-aldehyde and the amine of interest. This example shows how a mixture of both 6- and 7-membered ring compounds are obtained. The direct reductive amination can be accomplished by any suitable method. For the exemplary approach shown, the reductive amination is performed by first reacting an aqueous solution of the di-aldehyde and the amine of interest, for example, benzyl amine, for example, 40-50° C. for, for example, 18-42 hours; and then treating the reaction mixture with sodium cyanoborohydride at, for example, 40-50° C., for example, 18-42 hours. The resulting solution can be neutralized and dialyzed against, for example, phosphate buffered saline (PBS) or sterile water over an appropriately sized centrifugal filter to separate the analog from unreacted amine. Many different types of amine containing molecules could be added. Any of the 20 L or D alpha amino acids or other amino acids including those with beta or gamma spacing could be added. Benzyl amine or benzyl amine derivatives with substitution on the aromatic ring or the benzylic carbon or with more than a one carbon spacer between the amine and the aromatic ring, or with heteroatoms substituted into the carbon spacer separating the amine and the aromatic ring could be added. Heterocyclic benzyl amines, for example, furan, thiophene, pyrrole, imidazole, oxazole, isoxazole, thioazole, isothiazole, triazole, oxadiazole, thiadiazole, pyrazole, tetrazole, pyridine, diazine, triazine, tetrazine; or fused versions of any of the aromatic and heterocyclic rings, for example, benzothiophene, indole, isoindole, quinoline, or isoquinoline; or heterocyclic benzyl amine derivatives with substitution on the aromatic ring or the benzylic carbon or with more than a one carbon spacer between the amine and the aromatic ring; or with heteroatoms substituted into the carbon spacer separating the amine and the aromatic ring could be added. Aromatic amines, for example, aniline or derivatives of aniline, quinoline, or derivatives of quinoline, for example 2-aminoquinoline, 2-amino-8-hydroxy quinoline, nucleotides, for example, guanine, cytosine, adenine, or thymine, or nucleotide derivatives, for example, 2-amino-7H-purine-6-thiol could be added. Amine containing sugars, oligosaccharides, polysaccharides, or glucans could be added. Alkyl substituted amines, for example, molecules containing isobutyl amine, isopropyl amine, propyl amine, butyl amine, morpholine, piperidine, or piperazine could be added. Peglyated amines could also be added. Hydrazine and hydrazide containing molecules could also be added.

The approach shown below involves the installation of linker groups that will eventually be reacted with the molecule of interest to form analogs. Any suitable linker can be used. The exemplary approach shown below, for example, 1,3-diaminopropane followed by reacting the amino-derivatized glucan with an alkyl-linked bis N-hydroxy succinate (NHS) ester that will eventually be reacted with the amine-containing molecule. Diaminoalkane linkers of any other length could also be employed. Other alkyl-linked bis-electrophiles could also be used including, for example, sulfo-NHS esters, imidoesters, or maleimides. Non alkyl-linked coupling reagents could also be used including, for example, cyanogen bromide, cyanuric chloride, methyl phosphines, squarate esters, or 1,5-difluoro-2,4-dinitrobenzene. Briefly, this approach involves reacting the amino-derivatized glucan with an excess of an adipic acid derivative such as, for example, bis-NHS diester, in, for example, DMSO. The resulting activated β-glucan NHS ester can be isolated by precipitation with, for example, dioxane and further reacted with a basic aqueous solution of the amine-containing molecule. The final product can be once again isolated by dialysis against, for example, sterile PBS using an appropriately sized centrifugal filtration unit. One advantage of using the approach shown below involves the ability to use linkers of different lengths, thereby varying the distance between the glucan and the amine-containing molecule.

Below shows the coupling of NaIO₄ oxidized glucan with diaminopropane, activation and subsequent coupling with benzyl amine, one example of an amine-containing molecule.

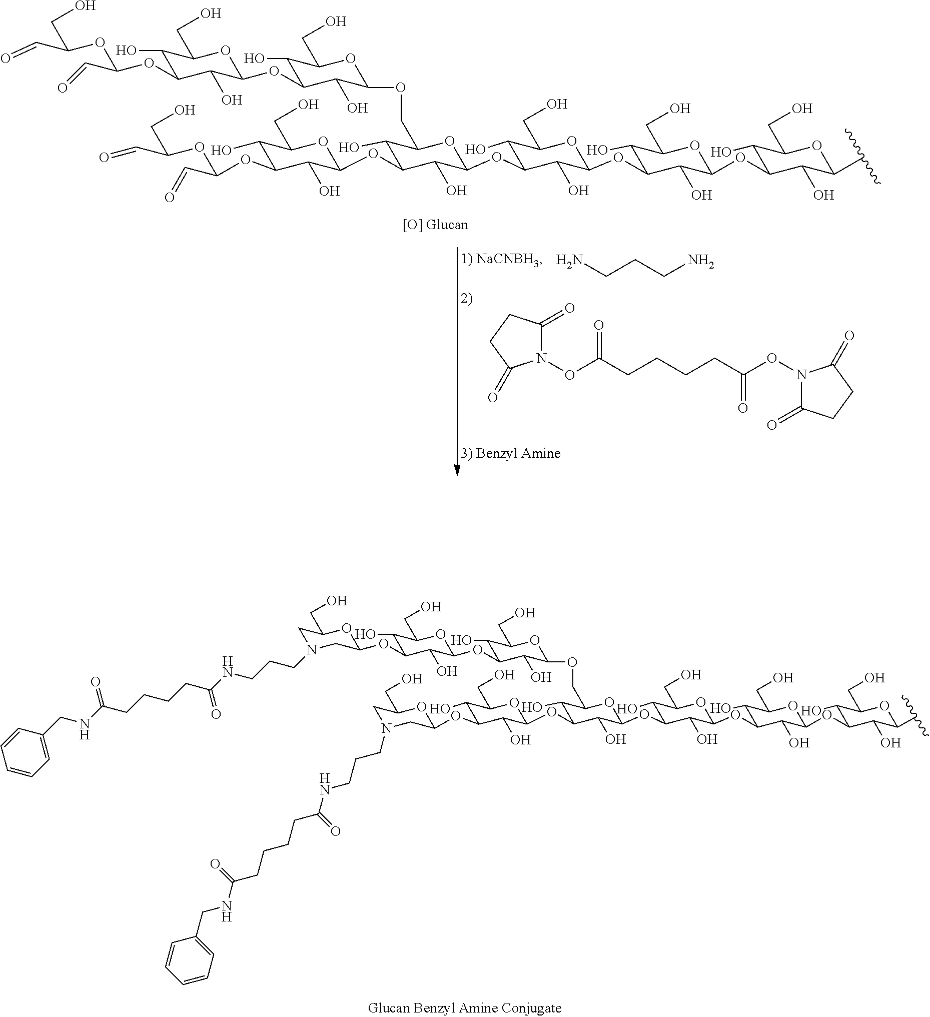 [SMILES: *C1OC(CO)C(O)C(OC2OC(CO)C(O)C(OC3OC(CO)C(O)C(OC4OC(COC5OC(CO)C(O)C(OC6OC(CO)C(O)C(OC(C=O)OC(C=O)CO)C6O)C5O)C(O)C(OC5OC(CO)C(O)C(OC(C=O)OC(C=O)CO)C5O)C4O)C3O)C2O)C1O ; NCCCN ; O=C(CCCCC(=O)ON1C(=O)CCC1=O)ON1C(=O)CCC1=O ; *C1OC(CO)C(O)C(OC2OC(CO)C(O)C(OC3OC(CO)C(O)C(OC4OC(COC5OC(CO)C(O)C(OC6OC(CO)C(O)C(OC7CN(CCCNC(=O)CCCCC(=O)NCc8ccccc8)CC(CO)O7)C6O)C5O)C(O)C(OC5OC(CO)C(O)C(OC6CN(CCCNC(=O)CCCCC(=O)NCc7ccccc7)CC(CO)O6)C5O)C4O)C3O)C2O)C1O]

The approach shown below utilizes the newly installed amine functionality resulting from the addition of diaminopropane to the di-aldehyde. Many types of amine reactive electrophiles can then be added, for example, reducing sugars, aldehydes, or ketones to form imines, which can then be reduced to form alkyl amines. Examples of reducing sugars that could be added include glucose to provide glucitol analogs, mannose to provide mannitol analogs, or galactose to provide galactitol analogs. Other reducing sugar containing molecules that could be added include allose, altrose, gulose, idose, and talose. In addition to monosaccharides; oligosaccharides, polysaccharides or glucans that contain these monosaccharide groups could also be added. Aldehydes bound to carbon based or heterocyclic based aromatic rings, for example, benzaldehyde or aldehyde substituted pyridine, imidazole, pyrazole, quinoline, isoquinoline or indole could also be added. Aldehydes bound to simple alkyl substituents could also be added, for example, methyl, ethyl, n-propyl, n-butyl, n-pentyl, cyclobutyl, cyclopentyl, cyclohexyl; or isomers of these. Aldehydes bound to hydroxyl or ether containing alkyl chains or rings could also be added. Other electrophiles could also be added that don't require reductive amination, for example, carboxylic acids, acid chlorides, or anhydrides to form amides; sulfonyl chlorides to form sulfonamides; alkyl or phenyl isocyanates to form ureas; and alkyl or phenyl thioisocyanates to form thioureas. Examples of carboxylic acids and acid chlorides include those attached to a phenyl, substituted phenyl, methyl, ethyl, propyl, pentyl, hexyl, cyclobutyl, cyclopentyl, cyclohexyl, oxygen or nitrogen containing alkyl, aromatic heterocycle, or non-aromatic heterocycle. Examples of sulfonyl chlorides include those attached to phenyl, substituted phenyl, methyl, ethyl, propyl, pentyl, hexyl, cyclobutyl, cyclopentyl, cyclohexyl, oxygen or nitrogen containing alkyl, an aromatic heterocycle, or a non-aromatic heterocycle.

Below shows the coupling of NaIO₄ oxidized glucan with diaminopropane and subsequent coupling with mannose, one example of a reducing sugar containing molecule.

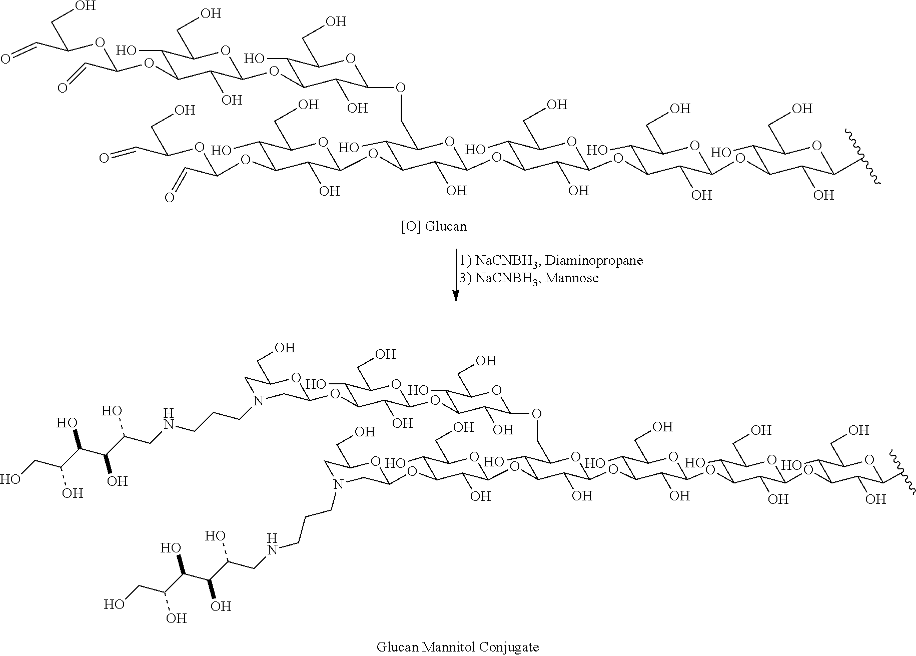

A second approach, also utilizing reductive amination, takes advantage of the reactive aldehyde functionality already present in the reducing end group. It is known that amines can be incorporated into this functionality. The reaction can be performed by reacting the glucan with the amine-containing molecule in the presence of sodium cyanoborohydride in either DMSO or an aqueous solution, as shown below. Similar to the first approach described immediately above, one can also form analogs in a sequential manner involving first coupling a linker amine, activating, and then coupling with the amine-containing molecule; or reacting the newly formed linker amine with electrophiles, for example, reducing sugars, aldehyde containing molecules, acid chlorides, anhydrides, sulfonyl chlorides, alkyl or phenyl isocyanates, or alkyl or phenyl thioisocyanates to form thioureas.

Below shows the reducing-end reductive amination of glucan with diaminopropane, one example of an amine-containing molecule.

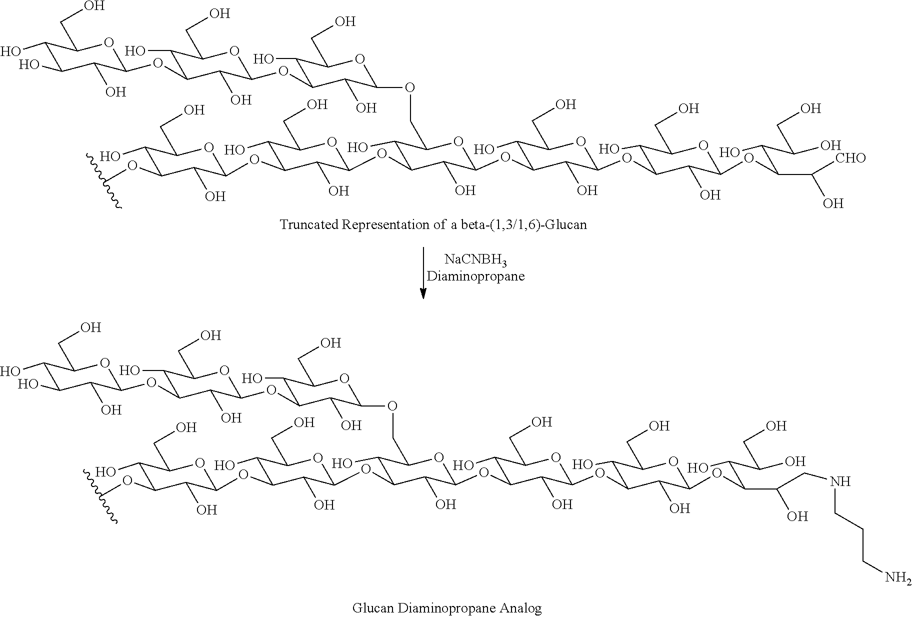 [SMILES: *OC1C(O)C(CO)OC(OC2C(O)C(CO)OC(OC3C(O)C(COC4OC(CO)C(O)C(OC5OC(CO)C(O)C(OC6OC(CO)C(O)C(O)C6O)C5O)C4O)OC(OC4C(O)C(CO)OC(OC5C(O)C(CO)OC(OC(C(O)C=O)C(O)C(O)CO)C5O)C4O)C3O)C2O)C1O ; *OC1C(O)C(CO)OC(OC2C(O)C(CO)OC(OC3C(O)C(COC4OC(CO)C(O)C(OC5OC(CO)C(O)C(OC6OC(CO)C(O)C(O)C6O)C5O)C4O)OC(OC4C(O)C(CO)OC(OC5C(O)C(CO)OC(OC(C(O)CNCCCN)C(O)C(O)CO)C5O)C4O)C3O)C2O)C1O]

A third approach is general and encompasses many potential ways to make an analog from β-glucan. This approach involves an initial alkylation of the hydroxyl residues located along the backbone or side chains of the β-glucan. Any suitable electrophile may be used for the alkylation reaction. The electrophile that is used can influence the types of subsequent coupling reactions that can be performed. Additionally, the alkylation product could also be an analog of interest. Examples of electrophiles include, for example, epichlorohydrin, chloroacetic acid, a halogen containing protected aldehyde, for example, dimethyl chloroacetal (2-chloro-1,1-dimethoxyethane), or a halogen-containing aminoalkane, for example the hydrobromide salt of 1-bromo-3-aminopropane. The use of chloroacetic acid, shown above, results in the installation of a reactive carboxylic acid moiety that can be directly reacted with an amine-containing molecule or an amine-containing linker such as those described above in connection with other conjugation approaches.

Below shows the alkylation of β-glucan with chloroacetic acid and subsequent coupling with diaminopropane.

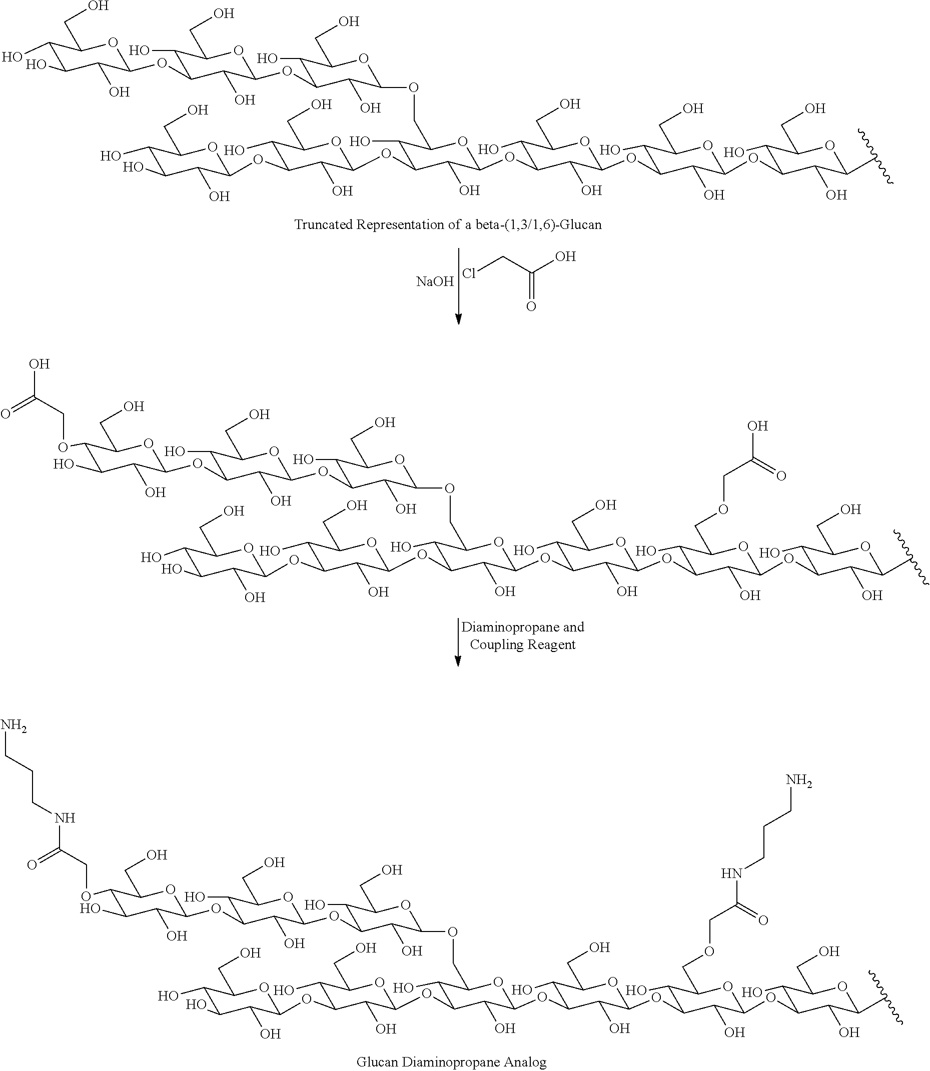

The use of halogen containing aminopropane, shown above, can feed into the same types of activation and coupling reactions using either the bis NHS ester linking groups and subsequent reaction with amine-containing molecules or into the reactions with different electrophile containing molecules, for example, reducing sugars, aldehyde containing molecules, acid chlorides, anhydrides, sulfonyl chlorides, alkyl or phenyl isocyanates, or alkyl or phenyl thioisocyanates.

Below shows the alkylation of β-glucan with 1-bromo-3-aminopropane.

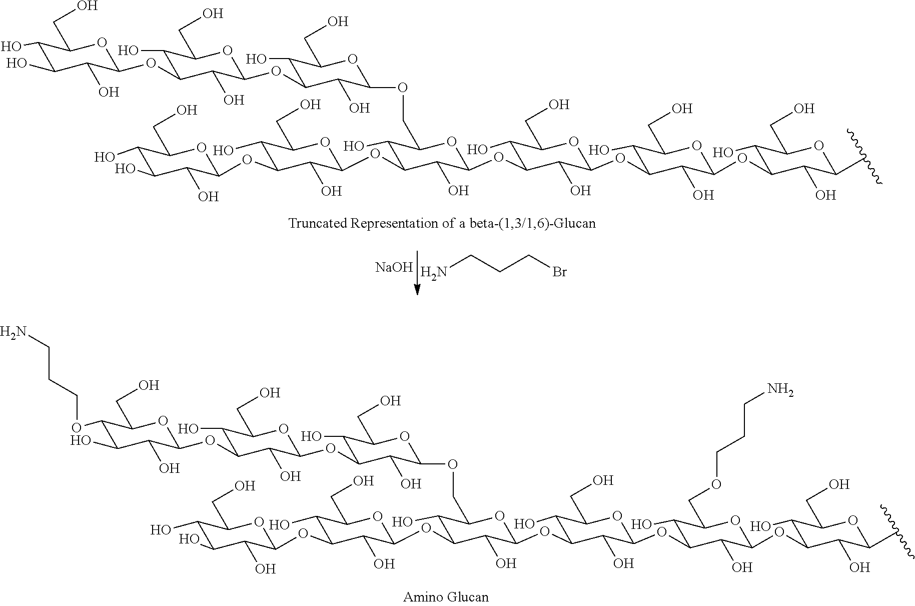 [SMILES: *C1OC(CO)C(O)C(OC2OC(CO)C(O)C(OC3OC(CO)C(O)C(OC4OC(COC5OC(CO)C(O)C(OC6OC(CO)C(O)C(OC7OC(CO)C(O)C(O)C7O)C6O)C5O)C(O)C(OC5OC(CO)C(O)C(OC6OC(CO)C(O)C(O)C6O)C5O)C4O)C3O)C2O)C1O ; "NaOH" ; NCCCBr ; *C1OC(CO)C(O)C(OC2OC(COCCCN)C(O)C(OC3OC(CO)C(O)C(OC4OC(COC5OC(CO)C(O)C(OC6OC(CO)C(O)C(OC7OC(CO)C(OCCCN)C(O)C7O)C6O)C5O)C(O)C(OC5OC(CO)C(O)C(OC6OC(CO)C(O)C(O)C6O)C5O)C4O)C3O)C2O)C1O]

A fourth approach uses a similar strategy to the carboxylic acid installation discussed immediately above. The primary hydroxyl group of glucose residues, including those found in β-glucan, can be readily oxidized to carboxylic acids when exposed to 2,2,6,6,-tetramethyl-1-piperidinyloxy (TEMPO) and sodium hypochlorite, resulting in the formation of glucoronic acid moieties, as shown below. The carboxylic acid can then be reacted as discussed above, either directly with an amine-containing molecule or first with an amino linker and then subsequently with an amine-containing molecules or with electrophile containing molecules.

Below shows the TEMPO-mediated oxidation of β-glucan to afford glucoronic acid functionalized glucan.

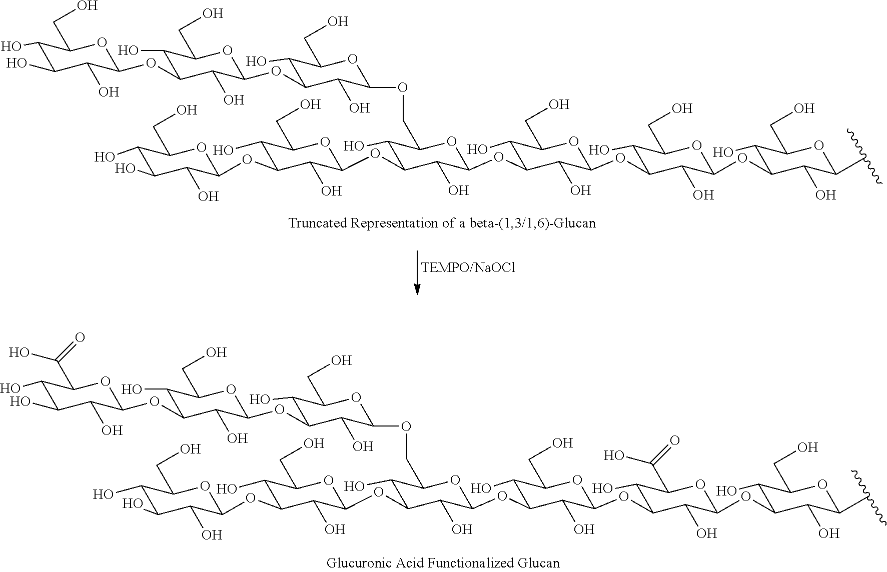 [SMILES: *C1OC(CO)C(O)C(OC2OC(CO)C(O)C(OC3OC(CO)C(O)C(OC4OC(COC5OC(CO)C(O)C(OC6OC(CO)C(O)C(OC7OC(CO)C(O)C(O)C7O)C6O)C5O)C(O)C(OC5OC(CO)C(O)C(OC6OC(CO)C(O)C(O)C6O)C5O)C4O)C3O)C2O)C1O ; "TEMPO/NaOCl" ; *C1OC(CO)C(O)C(OC2OC(C(=O)O)C(O)C(OC3OC(CO)C(O)C(OC4OC(COC5OC(CO)C(O)C(OC6OC(CO)C(O)C(OC7OC(C(=O)O)C(O)C(O)C7O)C6O)C5O)C(O)C(OC5OC(CO)C(O)C(OC6OC(CO)C(O)C(O)C6O)C5O)C4O)C3O)C2O)C1O]

Additional sources of glucan starting materials from which analogs can be derived include chemically modified yeast, mushroom, fungal, bacterial, or algal derived beta glucan. One example of how yeast beta glucan can be modified involves a Smith Degradation procedure which results in the shortening of the side chains by one glucose unit. The first step of the procedure involves a sodium periodate oxidation of the non-reducing terminal residues similar to the method described above. The second and third step of the procedure involves the reduction of the di-aldehyde moiety followed by hydrolysis with acid to afford a structure that has been truncated by one side chain residue.

Below shows the removal of single residues from the side chains of beta glucan, one example of a chemically modified glucan that can serve as the starting material for analog formation.

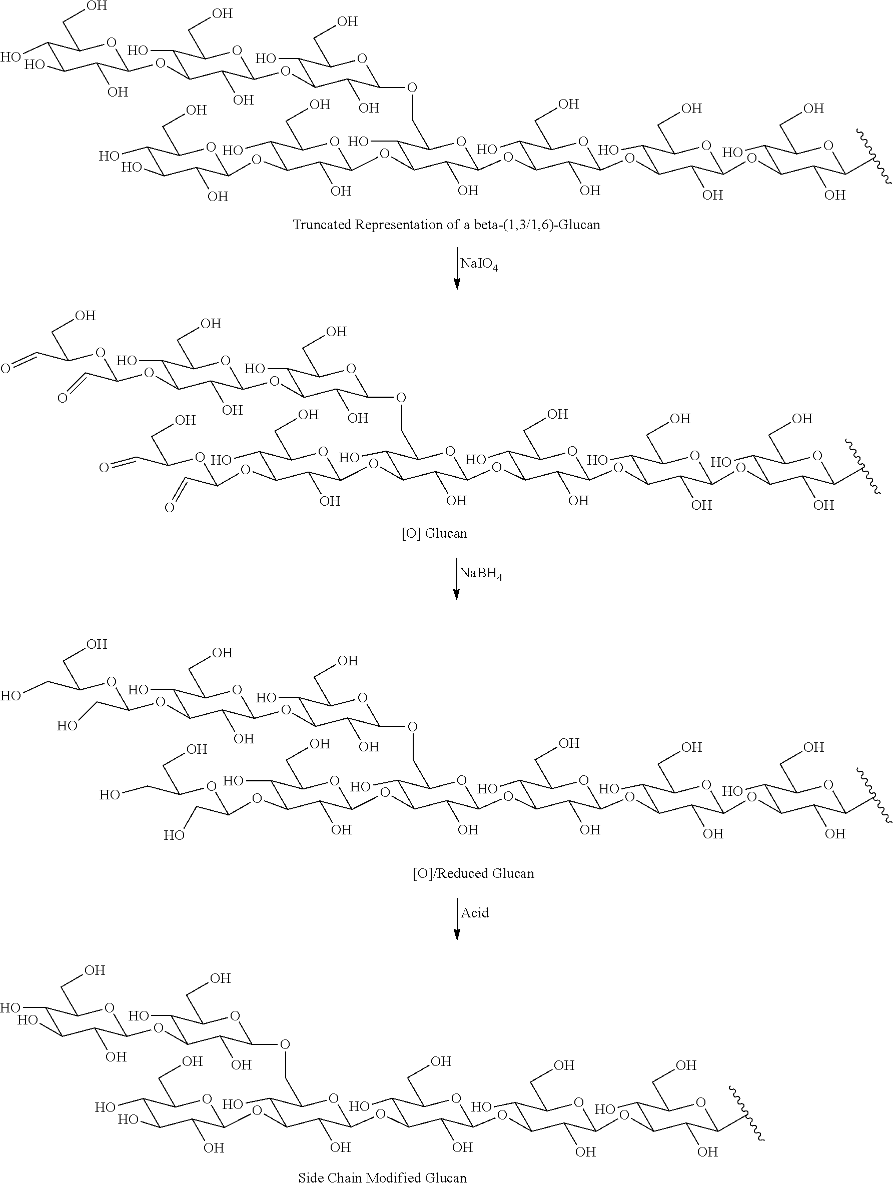

A second example involves the chemical modification of a beta glucan that normally exists as only a triple helix so that after being modified; it forms higher order aggregates of triple helices. The result of this modification is that one can take a glucan that exists only as an aggregate of 3 single chains, for example, scleroglucan, and reduce the frequency of branching such that it can now form aggregates with more than 3 single chains; similar to, for example, yeast glucan derived from Saccharomyces cerevisiae. The procedure involves a Smith Degradation procedure and is identical to that shown above. The result of this procedure is the removal of single branch point residues resulting in the production of material with a lower frequency of branching and an aggregation state higher than that exhibited by the starting glucan.

A third example of a chemically modified glucan that will be used as the starting material for analogs involves the isolation of an intermediate, labeled as [O]/Reduced Glucan, shown above. The specific compound is isolated following oxidation with NaIO₄ and reduction with NaBH₄. The result of this modification is the production of a glucan with glycol capped side chains and reducing ends.

The present invention is illustrated by the following examples. It is to be understood that the particular examples, materials, amounts, and procedures are to be interpreted broadly in accordance with the scope and spirit of the invention as set forth herein.

### EXAMPLES

All manipulations were performed in a sterile cell culture hood. Stock solutions of different Mw fractions of Saccharomyces cerevisiae R4 yeast glucan, either high molecular weight (HMW, weight average molecular weight (Mw)=360,000 daltons), medium molecular weight (MMW, Mw=150,000 daltons) or low molecular weight (LMW, Mw=10,000 daltons) in 0.011 M sodium citrate buffered 0.14 M saline were obtained from Biothera (Eagan, Minn.). The glucans were concentrated in multiple 15 mL capacity 3K or 10K molecular weight cut off (MWCO) AMICON centrifugal filters (Millipore Corp., Billerica, Mass.) or 10K MWCO MINIMATE tangential flow filtration devices (Pall Life Sciences, Ann Arbor, Mich.). The concentrates were brought into sterile water by performing three complete exchanges. Insoluble whole glucan particles (WGP) and soluble very high molecular weight (VHMW, Mw=900,000 daltons) from Saccharomyces cerevisiae were also obtained from Biothera. Commercially available glucans, including scleroglucan, dextran, laminarin, and barley glucan were dissolved in sterile water. The concentrations of the glucans were established by integration of the differential refractive index using high performance size exclusion chromatography or an anthrone assay. (reference: Bailey R W. The Reaction of Pentoses with Anthrone. Journal of Biological Chemistry 68:669-672, 1958)

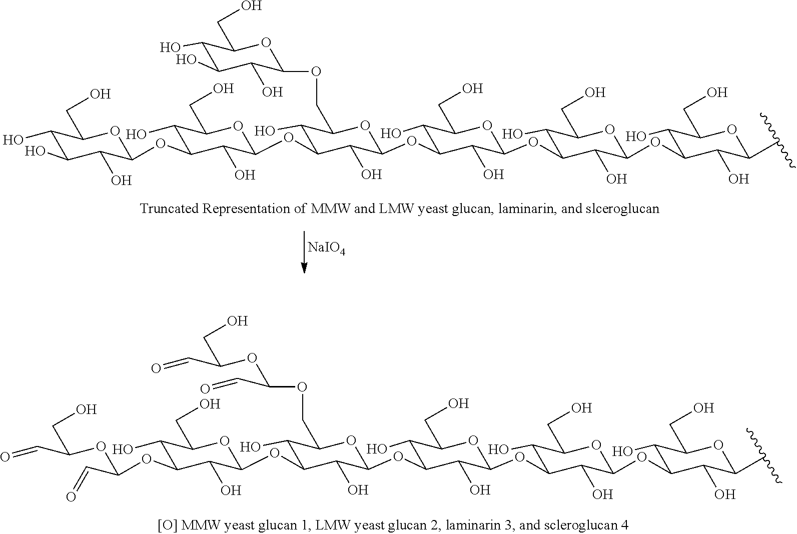

### Oxidized MMW Yeast Glucan 1, LMW Yeast Glucan 2, Scleroglucan 3, and Laminarin 4

### Oxidized MMW Yeast Glucan 1-L and 1-H

To an aqueous solution of MMW yeast glucan of known concentration was added an aqueous 25 mg/mL NaIO₄ solution to obtain a ratio of either 0.08 mg or 0.12 mg of NaIO₄ per mg of glucan. The final reaction concentration, with respect to glucan, was adjusted to 10 mg/mL with sterile water. The reactions were swirled and mixed to dissolve all reagent and the reactions was placed in the dark for 20 hours. The oxidized products, 1-L and 1-H, were carried directly into the reductive amination step without purification and correspond to 0.08 mg and 0.12 mg of NaIO₄ per mg of glucan respectively.

### Oxidized LMW Yeast Glucan 2-L and 2-H

To an aqueous solution of LMW yeast glucan of known concentration was added an aqueous 25 mg/mL NaIO₄ solution to obtain a ratio of either 0.12 or 0.16 mg of NaIO₄ per mg of glucan. The final reaction concentrations, with respect to glucan, were 4.5 mg/mL. The reactions were swirled and mixed to dissolve all reagent and the reactions were placed in the dark for 20 hours. The oxidized products, 2-L and 2-H, were carried directly into the reductive amination step without purification and correspond to 0.12 mg and 0.16 mg of NaIO₄ per mg of glucan respectively.

### Oxidized Laminarin 3-L, 3-M, and 3-H

To an aqueous solution of Laminarin of known concentration was added an aqueous 25 mg/mL NaIO₄ solution to obtain a ratio of either 0.12, 0.24, or 0.47 mg of NaIO₄ per mg of glucan. The final reaction concentrations, with respect to glucan, were adjusted to 10 mg/mL with sterile water. The reactions were swirled and mixed to dissolve all reagent and the reactions was placed in the dark for 20 hours. The oxidized products, 3-L, 3-M, and 3-H, were carried directly into the reductive amination step without purification and correspond to 0.12 mg, 0.24 mg, and 0.47 mg of NaIO₄ per mg of glucan respectively.

### Oxidized Scleroglucan 4

### Solubilization of Insoluble Scleroglucan

Insoluble sleroglucan (1.0 g), formic acid (99%, 84 mL), and a stir bar were combined in a threaded round bottom pressure vessel (150 mL). To the resulting solution, while stirring, was added triflouroacetic anhydride dropwise at 0° C. The flask was capped and the mixture was warmed to 65° C. and stirred at this temperature for 6 h and room temperature overnight. The resulting solution was then cooled to 0° C. and EtOH (160 mL) was added, at which point a white precipitate formed. The resulting suspension was warmed to room temperature over 2 h and stirred for 5 h at room temperature before dividing evenly into 50 mL centrifuge tubes. The suspension was centrifuged and the supernatant was discarded. The resulting pellet was washed twice more with an additional 50 mL EtOH each time. The final pellets were combined in a plastic bottle, water (150 mL) was added, and the pH of the solution was raised to 13 with NaOH (50%). The solution was swirled over 30 min at which point the pH was still at 13. The pH was then lowered to 5 with HCl. The slightly cloudy solution was divided evenly into 50 mL centrifuge tubes. The solution was centrifuged and the supernatant was 0.45 μm filtered. This material was then dialyzed into sterile water using 10K centrifugal filtration devices. The final retentates were sterile filtered through a 0.2 μm filter to provide soluble scleroglucan.

### Oxidation of Soluble Scleroglucan

To an aqueous solution of soluble scleroglucan of known concentration was added an aqueous 25 mg/mL NaIO₄ solution to obtain a ratio of 0.14 mg of NaIO₄ per mg of glucan. The final reaction concentration, with respect to glucan, was 11 mg/mL. The reaction was swirled and mixed to dissolve all reagent and the reaction was placed in the dark for 20 hours. The oxidized product was carried directly into the reductive amination step without purification.

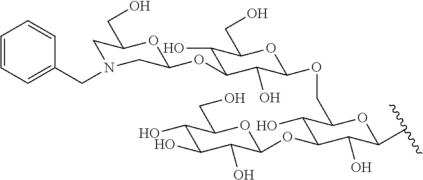 [SMILES: *C1OC(COC2OC(CO)C(O)C(OC3CN(Cc4ccccc4)CC(CO)O3)C2O)C(O)C(OC2OC(CO)C(O)C(O)C2O)C1O]

### Benzyl Amine MMW Yeast Glucan Analog BT-1222

To a 15 mL tube containing [O] MMW yeast glucan 1-H (5 mL of a 10 mg/mL aqueous solution) was added benzyl amine (0.05 mL). The reaction was sealed and placed in a 40° C. water bath for 18 hours. The reaction was removed from the water bath and cooled to 0° C. To the resulting solution was added NaCNBH₃ (100 mg in 1.0 mL of dPBS) and the reaction was swirled to mix, sealed, and placed in a 40° C. water bath for 18 hours. The reaction mixture was next dialyzed into sterile water using 3K centrifugal filtration units. The concentrated material was diluted into water and sterile filtered through a 0.2 μm syringe filter to afford an aqueous solution containing 20 mg of BT-1222. Elemental analysis by combustion on lyophilized material gave a % N of 0.39%. This translates to 4.6 mol % of benzyl amine with respect to the individual glucose monomers of the glucan.

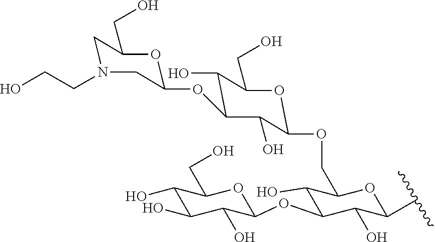 [SMILES: *C1OC(COC2OC(CO)C(O)C(OC3CN(CCO)CC(CO)O3)C2O)C(O)C(OC2OC(CO)C(O)C(O)C2O)C1O]

### Ethanolamine MMW Yeast Glucan Analog BT-1202

The procedure used to synthesize BT-1222 was repeated using ethanolamine (0.05 mL) and [O] MMW yeast glucan 1-H (50 mg) to afford 38 mg of BT-1202. Elemental analysis by combustion gave a % N of 0.34%, which translates to 3.5 mol % ethanolamine incorporation.

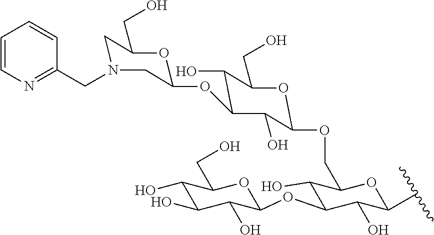 [SMILES: *C1OC(COC2OC(CO)C(O)C(OC3CN(Cc4ccccn4)CC(CO)O3)C2O)C(O)C(OC2OC(CO)C(O)C(O)C2O)C1O]

### 2-Aminomethyl Pyridine MMW Yeast Glucan Analog BT-1204

The procedure used to synthesize BT-1222 was repeated using 2-aminomethyl pyridine (50 mg) and [O] MMW yeast glucan 1-H (50 mg) to afford 37 mg of BT-1204. Elemental analysis by combustion gave a % N of 0.54%, which translates to 3.2 mol % 2-aminomethyl pyridine incorporation.

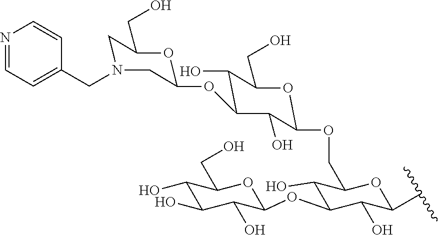 [SMILES: *C1OC(COC2OC(CO)C(O)C(OC3CN(Cc4ccncc4)CC(CO)O3)C2O)C(O)C(OC2OC(CO)C(O)C(O)C2O)C1O]

### 4-Aminomethyl pyridine MMW Yeast Glucan Analog BT-1205

The procedure used to synthesize BT-1222 was repeated using 4-aminomethyl pyridine (50 mg) and [O] MMW yeast glucan 1-H (50 mg) to afford 38 mg of BT-1205. Elemental analysis by combustion gave a % N of 0.64%, which translates to 3.8 mol % 4-aminomehtyl pyridine incorporation.

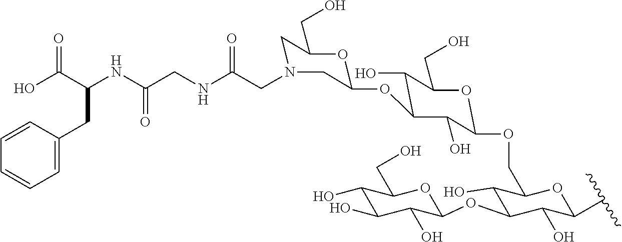 [SMILES: *C1OC(COC2OC(CO)C(O)C(OC3CN(CC(=O)NCC(=O)N[C@@H](Cc4ccccc4)C(=O)O)CC(CO)O3)C2O)C(O)C(OC2OC(CO)C(O)C(O)C2O)C1O]

### PheGlyGly MMW Yeast Glucan Analog BT-1206

The procedure used to synthesize BT-1222 was repeated using the tripeptide PheGlyGly (50 mg) and [O] MMW yeast glucan 1-H (50 mg) to afford 42 mg of BT-1206. Elemental analysis by combustion gave a % N of 0.97%, which translates to 4.2 mol % PheGlyGly incorporation.

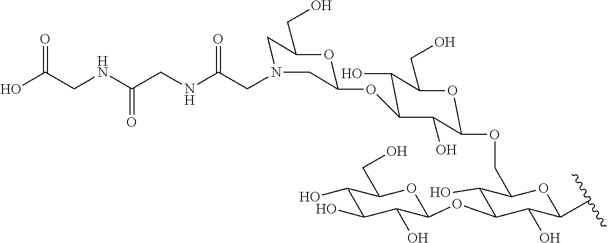 [SMILES: *C1OC(COC2OC(CO)C(O)C(OC3CN(CC(=O)NCC(=O)NCC(=O)O)CC(CO)O3)C2O)C(O)C(OC2OC(CO)C(O)C(O)C2O)C1O]

### GlyGlyGly MMW Yeast Glucan Analog BT-1207

The procedure used to synthesize BT-1222 was repeated using the tripeptide GlyGlyGly (50 mg) and [O] MMW yeast glucan 1-H (50 mg) to afford 44 mg of BT-1207. Elemental analysis by combustion gave a % N of 1.02%, which translates to 3.9 mol % GlyGlyGly incorporation.

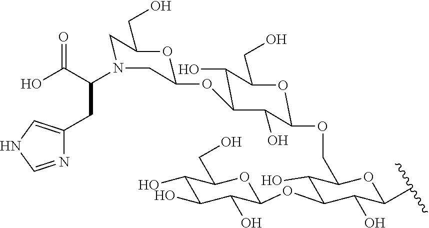 [SMILES: *C1OC(COC2OC(CO)C(O)C(OC3CN([C@@H](Cc4c[nH]cn4)C(=O)O)CC(CO)O3)C2O)C(O)C(OC2OC(CO)C(O)C(O)C2O)C1O]

### Histidine MMW Yeast Glucan Analog BT-1218

The procedure used to synthesize BT-1222 was repeated using histidine (50 mg) and [O] MMW yeast glucan 1-H (50 mg) to afford 42 mg of BT-1218. Elemental analysis by combustion gave a % N of 0.69%, which translates to 2.7 mol % histidine incorporation.

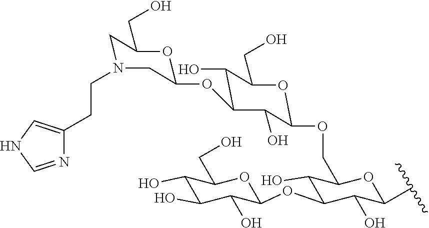 [SMILES: *C1OC(COC2OC(CO)C(O)C(OC3CN(CCc4c[nH]cn4)CC(CO)O3)C2O)C(O)C(OC2OC(CO)C(O)C(O)C2O)C1O]

### Histamine MMW Yeast Glucan Analog BT-1219

The procedure used to synthesize BT-1222 was repeated using histamine (50 mg) and [O] MMW yeast glucan 1-H (50 mg) to afford 32 mg of BT-1219. Elemental analysis by combustion gave a % N of 1.31%, which translates to 5.2 mol % histamine incorporation.

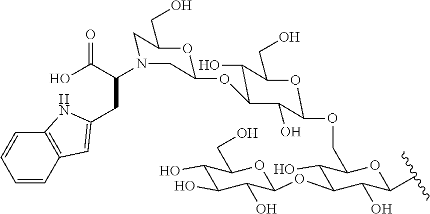 [SMILES: *C1OC(COC2OC(CO)C(O)C(OC3CN([C@@H](Cc4cc5ccccc5[nH]4)C(=O)O)CC(CO)O3)C2O)C(O)C(OC2OC(CO)C(O)C(O)C2O)C1O]

### Tryptophan MMW Yeast Glucan Analog BT-1220

The procedure used to synthesize BT-1222 was repeated using tryptophan (50 mg) and [O] MMW yeast glucan 1-H (50 mg) to afford 26 mg of BT-1220. Elemental analysis by combustion gave a % N of 0.95%, which translates to 5.8 mol % tryptophan incorporation.

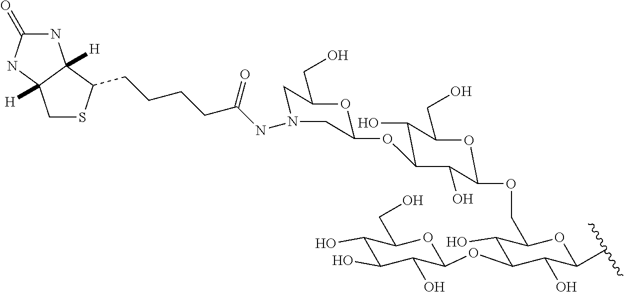

### Biotin MMW Yeast Glucan Analog BT-1221

The procedure used to synthesize BT-1222 was repeated using biotin hydrazide (50 mg) and [O] MMW yeast glucan 1-H (50 mg) to afford 31 mg of BT-1221. Elemental analysis by combustion gave a % N of 1.88%, which translates to 5.9 mol % biotin incorporation.

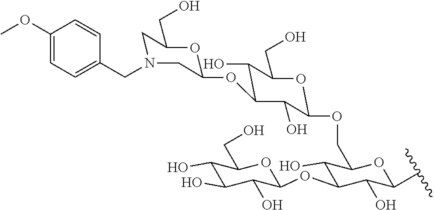 [SMILES: *C1OC(COC2OC(CO)C(O)C(OC3CN(Cc4ccc(OC)cc4)CC(CO)O3)C2O)C(O)C(OC2OC(CO)C(O)C(O)C2O)C1O]

### 4-Methoxy Benzyl Amine MMW Yeast Glucan Analog BT-1223

The procedure used to synthesize BT-1222 was repeated using 4-methoxy benzyl amine (50 mg) and [O] MMW yeast glucan 1-H (50 mg) to afford 12 mg of BT-1223. Elemental analysis by combustion gave a % N of 0.38%, which translates to 4.5 mol % 4-methoxy benzyl amine incorporation.

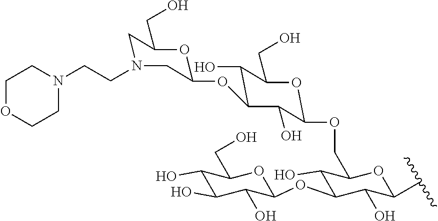 [SMILES: *C1OC(COC2OC(CO)C(O)C(OC3CN(CCN4CCOCC4)CC(CO)O3)C2O)C(O)C(OC2OC(CO)C(O)C(O)C2O)C1O]

### 4-(2-Aminoethyl)-Morpholine MMW Yeast Glucan Analog BT-1236

The procedure used to synthesize BT-1222 was repeated using 4-(2-aminoethyl)-morpholine (50 mg) and [O] MMW yeast glucan 1-H (50 mg) to afford 34 mg of BT-1236. Elemental analysis by combustion gave a % N of 0.52%, which translates to 3.1 mol % 4-(2-aminoethyl)-morpholine incorporation.

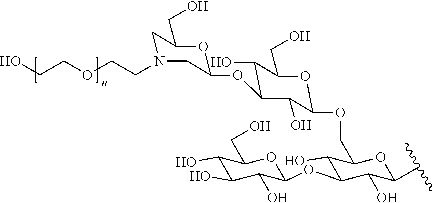

### Polyethylene Glycol (PEG) MMW Yeast Glucan Analog BT-1237

The procedure used to synthesize BT-1222 was repeated using 5,000 Mw amine functionalized PEG (300 mg) and [O] MMW yeast glucan 1-H (50 mg) to afford 31 mg of BT-1237. Elemental analysis by combustion gave a % N of 0.2%, which translates to 7.5 mol % amino-PEG incorporation.

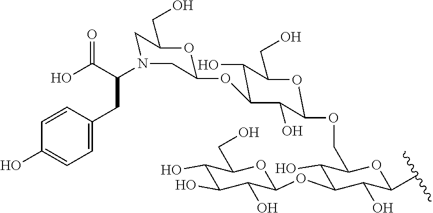 [SMILES: *C1OC(COC2OC(CO)C(O)C(OC3CN([C@@H](Cc4ccc(O)cc4)C(=O)O)CC(CO)O3)C2O)C(O)C(OC2OC(CO)C(O)C(O)C2O)C1O]

### Tyrosine MMW Yeast Glucan Analog BT-1297

The procedure used to synthesize BT-1222 was repeated using tyrosine (50 mg) and [O] MMW yeast glucan 1-H (50 mg) to afford 39 mg of BT-1297. Elemental analysis by combustion gave a % N of 0.28%, which translates to 1.6 mol % tyrosine incorporation.

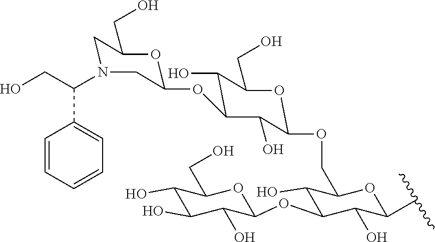 [SMILES: *C1OC(COC2OC(CO)C(O)C(OC3CN([C@@H](CO)c4ccccc4)CC(CO)O3)C2O)C(O)C(OC2OC(CO)C(O)C(O)C2O)C1O]

### R-(−)-2-Phenyl Glycinol MMW Yeast Glucan Analog BT-1298

The procedure used to synthesize BT-1222 was repeated using R-(−)-2-phenyl glycinol (50 mg) and [O] MMW yeast glucan 1-H (50 mg) to afford 14 mg of BT-1298. Elemental analysis by combustion gave a % N of 0.39%, which translates to 4.6 mol % R-(−)-2-phenyl glycinol incorporation.

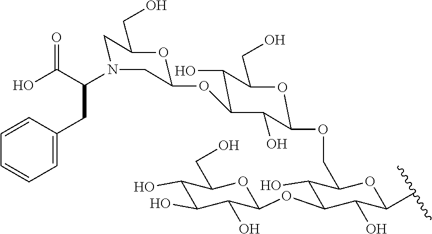 [SMILES: *C1OC(COC2OC(CO)C(O)C(OC3CN([C@@H](Cc4ccccc4)C(=O)O)CC(CO)O3)C2O)C(O)C(OC2OC(CO)C(O)C(O)C2O)C1O]

### Phenylalanine MMW Yeast Glucan Analog BT-1299

The procedure used to synthesize BT-1222 was repeated using phenylalanine (50 mg) and [O] MMW yeast glucan 1-H (50 mg) to afford 42 mg of BT-1299. Elemental analysis by combustion gave a % N of 0.31%, which translates to 3.7 mol % phenylalanine incorporation.

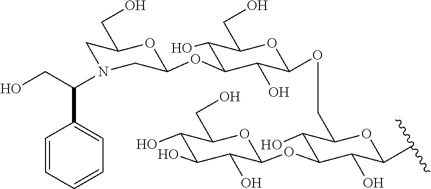

### S-(+)-2-Phenyl Glycinol MMW Yeast Glucan Analog BT-1300

The procedure used to synthesize BT-1222 was repeated using S-(+)-2-phenyl glycinol (50 mg) and [O] MMW yeast glucan 1-H (50 mg) to afford 22 mg of BT-1300. Elemental analysis by combustion gave a % N of 0.41%, which translates to 4.9 mol % R-(−)-2-phenyl glycinol incorporation.

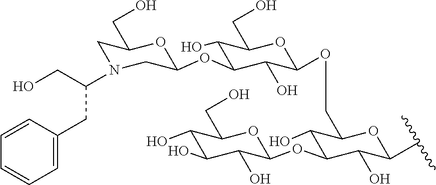

### D-(+)-2-Amino-3-Phenyl-1-Propanol MMW Yeast Glucan Analog BT-1301

The procedure used to synthesize BT-1222 was repeated using D-(+)-2-amino-3-phenyl-1-propanol (50 mg) and [O] MMW yeast glucan 1-H (50 mg) to afford 29 mg of BT-1301. Elemental analysis by combustion gave a % N of 0.3%, which translates to 3.6 mol % D-(+)-2-amino-3-phenyl-1-propanol incorporation.

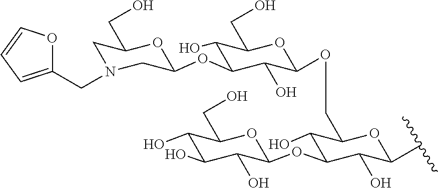 [SMILES: *C1OC(COC2OC(CO)C(O)C(OC3CN(Cc4ccco4)CC(CO)O3)C2O)C(O)C(OC2OC(CO)C(O)C(O)C2O)C1O]

### Furfurylamine MMW Yeast Glucan Analog BT-1302

The procedure used to synthesize BT-1222 was repeated using furfurylamine (50 mg) and [O] MMW yeast glucan 1-H (50 mg) to afford 31 mg of BT-1302. Elemental analysis by combustion gave a % N of 0.47%, which translates to 5.5 mol % furfurylamine incorporation.

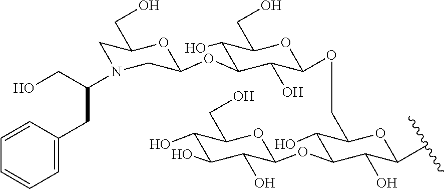

### L-(−)-2-Amino-3-Phenyl-1-Propanol MMW Yeast Glucan Analog BT-1303

The procedure used to synthesize BT-1222 was repeated using L-(−)-2-amino-3-phenyl-1-propanol (50 mg) and [O] MMW yeast glucan 1-H (50 mg) to afford 29 mg of BT-1303. Elemental analysis by combustion gave a % N of 0.32%, which translates to 3.8 mol % L-(−)-2-amino-3-phenyl-1-propanol incorporation.

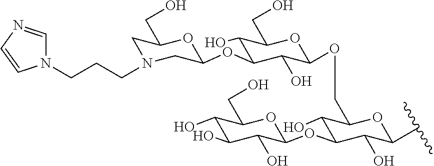 [SMILES: *C1OC(COC2OC(CO)C(O)C(OC3CN(CCCn4ccnc4)CC(CO)O3)C2O)C(O)C(OC2OC(CO)C(O)C(O)C2O)C1O]

### 3-Aminopropyl Imidazole MMW Yeast Glucan Analog BT-1304

The procedure used to synthesize BT-1222 was repeated using 3-aminopropyl imidazole (50 mg) and [O] MMW yeast glucan 1-H (50 mg) to afford 33 mg of BT-1304. Elemental analysis by combustion gave a % N of 1.13%, which translates to 4.5 mol % 3-aminopropyl imidazole incorporation.

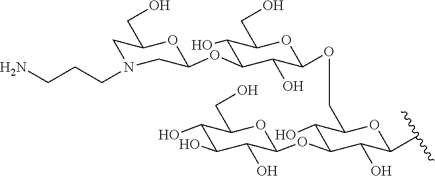 [SMILES: *C1OC(COC2OC(CO)C(O)C(OC3CN(CCCN)CC(CO)O3)C2O)C(O)C(OC2OC(CO)C(O)C(O)C2O)C1O]

### Diaminopropane MMW Yeast Glucan Analog BT-1253

The procedure used to synthesize BT-1222 was repeated using 1,3-diaminopropane and [O] MMW yeast glucan 1-H (400 mg) to afford 200 mg of BT-1253. Elemental analysis by combustion gave a % N of 0.81%, which translates to 4.7 mol % 1,3-diaminopropane incorporation. The presence of the primary amine was confirmed by the Thermo Scientific Pierce Fluoraldehyde Protein/Peptide (OPA) assay and was found to contain 3.5 mol % primary amine. This procedure was repeated with a second sample of MMW Yeast Glucan (325 mg) to afford an additional sample of BT-1253 with 0.69 mol % nitrogen.

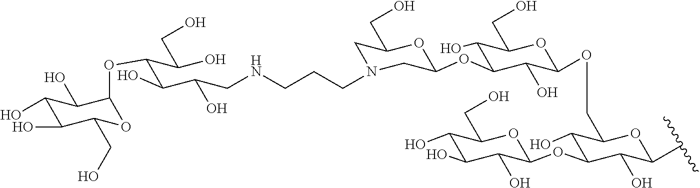 [SMILES: *C1OC(COC2OC(CO)C(O)C(OC3CN(CCCNCC(O)C(O)C(OC4OC(CO)C(O)C(O)C4O)C(O)CO)CC(CO)O3)C2O)C(O)C(OC2OC(CO)C(O)C(O)C2O)C1O]

### Maltose 1,3-Diaminopropane MMW Yeast Glucan Analog BT-1238

To a solution of diaminopropane MMW yeast glucan analog BT-1253 (40 mg) in sterile water (3.3 mL) and methanol (1.0 mL) was added maltose (78 mg) followed by borane pyridine complex (0.15 mL, 8 M in THF). The resulting solution was vortexed and placed at 50° C. for three days. After cooling to rt, the reactions mixture was washed with dichloromethane in a separatory funnel and the water layer was dialyzed into sterile water over 3K centrifugal filtration devices to afford 40 mg of BT-1238. The loss of primary amine and proof of reductive amination was confirmed by an OPA assay. BT-1238 contained 0.3 mol % primary amine, indicating a 92% conversion from the 3.5 mol % primary amine present in the starting material, BT-1253. Elemental analysis by combustion gave a % N of 0.56% for BT-1238, which translates to 3.5 mol % incorporation.

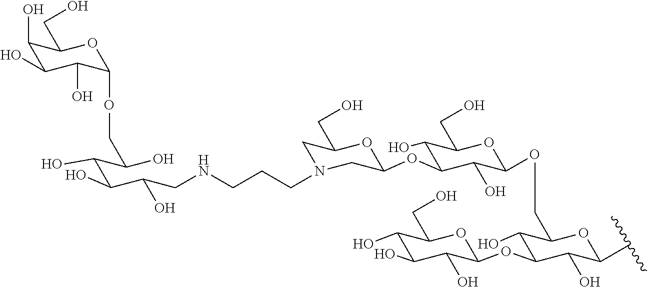 [SMILES: *C1OC(COC2OC(CO)C(O)C(OC3CN(CCCNCC(O)C(O)C(O)C(O)COC4OC(CO)C(O)C(O)C4O)CC(CO)O3)C2O)C(O)C(OC2OC(CO)C(O)C(O)C2O)C1O]

### Melbiose 1,3-Diaminopropane MMW Yeast Glucan Analog BT-1239

The procedure used to synthesize BT-1238 was repeated using melbiose (78 mg) and diaminopropane MMW yeast glucan analog BT-1253 (40 mg) to afford 35 mg of BT-1239. The loss of primary amine and proof of reductive amination was confirmed by an OPA assay. BT-1239 contained less than 0.1 mol % primary amine, indicating a complete conversion from the 3.5 mol % primary amine present in the starting material, BT-1253. Elemental analysis by combustion gave a % N of 0.71% for BT-1239, which translates to 4.5 mol % incorporation.

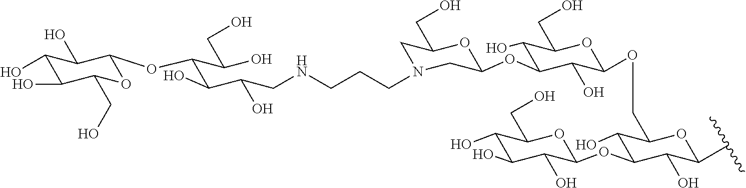 [SMILES: *C1OC(COC2OC(CO)C(O)C(OC3CN(CCCNCC(O)C(O)C(OC4OC(CO)C(O)C(O)C4O)C(O)CO)CC(CO)O3)C2O)C(O)C(OC2OC(CO)C(O)C(O)C2O)C1O]

### Cellobiose 1,3-Diaminopropane MMW Yeast Glucan Analog BT-1240

The procedure used to synthesize BT-1238 was repeated using cellobiose (78 mg) and diaminopropane MMW yeast glucan analog BT-1253 (40 mg). The workup procedure was altered and instead of washing with dichloromethane, the product was precipitated from the reaction mixture with ice cold ethanol and recovered by centrifugation. The pellet was washed with additional ethanol and dialyzed into sterile water over 3K centrifugal filtration devices to afford 40 mg of BT-1240. The loss of primary amine and proof of reductive amination was confirmed by an OPA assay. BT-1240 contained 0.27 mol % primary amine, indicating a 92% conversion from the 3.5 mol % primary amine present in the starting material, BT-1253. Elemental analysis by combustion gave a % N of 0.78% for BT-1240, which translates to 5.0 mol % incorporation.

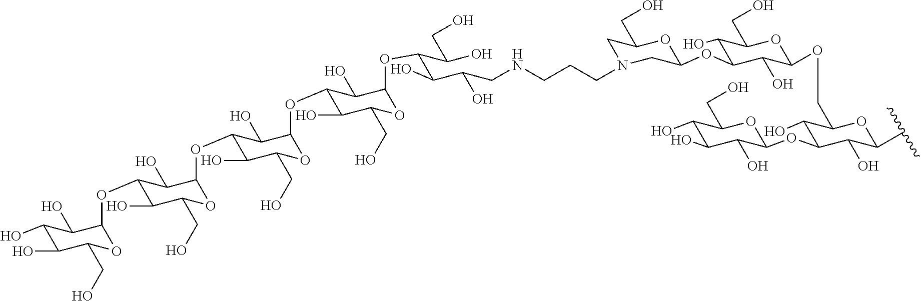 [SMILES: *C1OC(COC2OC(CO)C(O)C(OC3CN(CCCNCC(O)C(O)C(OC4OC(CO)C(O)C(OC5OC(CO)C(O)C(OC6OC(CO)C(O)C(OC7OC(CO)C(O)C(O)C7O)C6O)C5O)C4O)C(O)CO)CC(CO)O3)C2O)C(O)C(OC2OC(CO)C(O)C(O)C2O)C1O]

### Maltopentaose 1,3-Diaminopropane MMW Yeast Glucan Analog BT-1241

The procedure used to synthesize BT-1240 was repeated using maltopentaose (108 mg) and diaminopropane MMW yeast glucan analog BT-1253 (40 mg) to afford 40 mg of BT-1241. The loss of primary amine and proof of reductive amination was confirmed by an OPA assay. BT-1241 contained 0.21 mol % primary amine, indicating a 94% conversion from the 3.5 mol % primary amine present in the starting material, BT-1253. Elemental analysis by combustion gave a % N of 0.51% for BT-1241, which translates to 3.5 mol % incorporation.

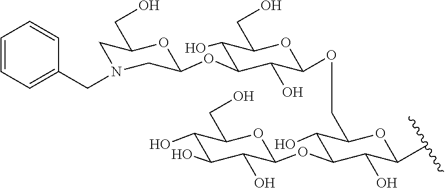 [SMILES: *C1OC(COC2OC(CO)C(O)C(OC3CN(Cc4ccccc4)CC(CO)O3)C2O)C(O)C(OC2OC(CO)C(O)C(O)C2O)C1O]

### Benzyl Amine LMW Yeast Glucan Analogs BT-1273 and BT-1274

The procedure used to synthesize BT-1222 was repeated using benzyl amine and [O] LMW yeast glucan 2-L (40 mg) and 2-H (40 mg) to afford 26 mg of BT-1273 and 14 mg of BT-1274 respectively. Elemental analysis by combustion gave a % N of 0.18% and 0.37% respectively, which translates to 2.1 mol % benzyl amine for BT-1273 and 4.4 mol % benzyl amine for BT-1274.

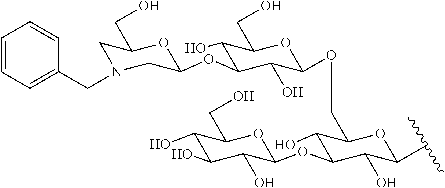 [SMILES: *C1OC(COC2OC(CO)C(O)C(OC3CN(Cc4ccccc4)CC(CO)O3)C2O)C(O)C(OC2OC(CO)C(O)C(O)C2O)C1O]

### Benzyl Amine Laminarin Analogs BT-1228, BT-1231, and BT-1234

The procedure used to synthesize BT-1222 was repeated using benzyl amine (50 mg) and [O] laminarin 3-L (50 mg), 3-M (50 mg), and 3-H (50 mg) to afford 29 mg of BT-1228, 20 mg of BT-1231, and 7 mg of BT-1234 respectively. Elemental analysis by combustion gave a % N of 0.21%, 0.59% and 0.92% respectively, which translates to 2.5 mol % benzyl amine for BT-1228, 7.0 mol % benzyl amine for BT-1231, and 11.1 mol % benzyl amine for BT-1234.

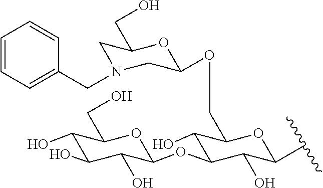 [SMILES: *C1OC(COC2CN(Cc3ccccc3)CC(CO)O2)C(O)C(OC2OC(CO)C(O)C(O)C2O)C1O]

### Benzyl Amine Scleroglucan Analog BT-1272

The procedure used to synthesize BT-1222 was repeated using benzyl amine and [O] scleroglucan 4 (50 mg) to afford 8 mg of BT-1272. Elemental analysis by combustion gave a % N of 0.5%, which translates to 5.9 mol % benzyl amine incorporation.

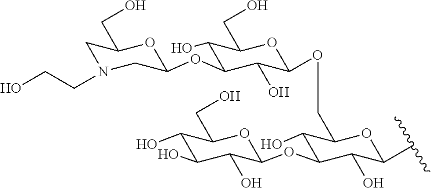 [SMILES: *C1OC(COC2OC(CO)C(O)C(OC3CN(CCO)CC(CO)O3)C2O)C(O)C(OC2OC(CO)C(O)C(O)C2O)C1O]

### Ethanolamine Laminarin Analogs BT-1227, BT-1230, and BT-1233

The procedure used to synthesize BT-1222 was repeated using ethanolamine (50 mg) and [O] laminarin 3-L (50 mg), 3-M (50 mg), and 3-H (50 mg) to afford 19 mg of BT-1227, 17 mg of BT-1230, and 21 mg of BT-1233 respectively. Elemental analysis by combustion gave a % N of 0.21%, 0.53% and 1.31% respectively, which translates to 2.4 mol % ethanolamine incorporation for BT-1227, 7.0 mol % ethanolamine incorporation for BT-1230, and 15.4 mol % ethanolamine incorporation for BT-1233.

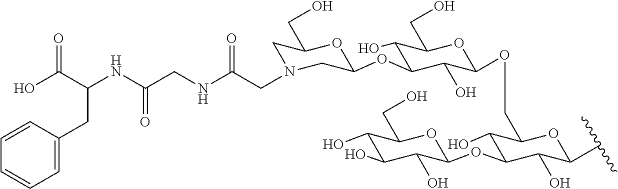 [SMILES: *C1OC(COC2OC(CO)C(O)C(OC3CN(CC(=O)NCC(=O)NC(Cc4ccccc4)C(=O)O)CC(CO)O3)C2O)C(O)C(OC2OC(CO)C(O)C(O)C2O)C1O]

### Tripeptide Laminarin Analogs BT-1229, BT-1232, and BT-1235

The procedure used to synthesize BT-1222 was repeated using the tripeptide PhePheGly and [O] laminarin 3-L (50 mg), 3-M (50 mg), and 3-H (50 mg) to afford 21 mg of BT-1229, 24 mg of BT-1232, and 24 mg of BT-1235 respectively. Elemental analysis by combustion gave a % N of 0.68%, 1.31% and 1.54% respectively, which translates to 2.7 mol % PhePheGly incorporation for BT-1229, 5.4 mol % PhePheGly incorporation for BT-1232, and 6.5 mol % PhePheGly incorporation for BT-1235.

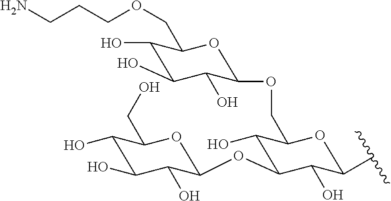 [SMILES: *C1OC(COC2OC(COCCCN)C(O)C(O)C2O)C(O)C(OC2OC(CO)C(O)C(O)C2O)C1O]

### Propyl Amine MMW Yeast Glucan Analog BT-1267 and BT-1268

To a solution of MMW yeast glucan (1 g) in sterile water (12 mL) at 0° C. with stirring was added aqueous NaOH (8 mL, 6.17 M). After stirring 20 min at 0° C., 1-bromo-3-aminopropane hydrobromide (1.4 g) was added and the resulting solution was stirred at rt for 3 h. The reaction was neutralized with HCl and the product was precipitated with ice cold ethanol and recovered by centrifugation. The pellet was washed with additional ethanol and dialyzed into sterile water over 3K centrifugal filtration devices to afford 0.87 g of BT-1267. The presence of the primary amine was measured by an OPA assay and was found to contain 3.5 mol % primary amine. This procedure was repeated on MMW yeast glucan (2 g) at twice the scale to afford 1.3 g of BT-1268. Elemental analysis by combustion gave a % N of 0.28%, which translates to 3.3 mol % propyl amine incorporation for BT-1268. The presence of the primary amine was confirmed by an OPA assay and was found to contain 3.1 mol % primary amine.

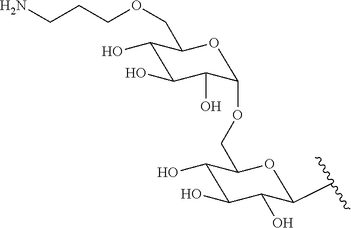 [SMILES: *C1OC(COC2OC(COCCCN)C(O)C(O)C2O)C(O)C(O)C1O]

### Propyl Amine Dextran Analog BT-1266

The procedure used to synthesize BT-1268 was repeated using dextran to afford BT-1266. Elemental analysis by combustion gave a % N of 0.14%, which translates to 1.6 mol % propyl amine incorporation for BT-1266. The presence of the primary amine was confirmed by an OPA assay and was found to contain 2.6 mol % primary amine.

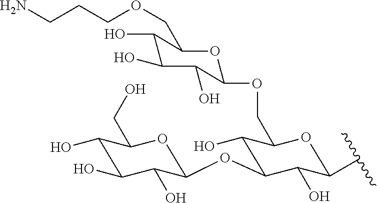 [SMILES: *C1OC(COC2OC(COCCCN)C(O)C(O)C2O)C(O)C(OC2OC(CO)C(O)C(O)C2O)C1O]

### Propyl Amine LMW Yeast Glucan Analog BT-1276

The procedure used to synthesize BT-1268 was repeated using LMW yeast glucan (256 mg) to afford 180 mg of BT-1276. Elemental analysis by combustion gave a % N of 0.27%, which translates to 3.2 mol % propyl amine incorporation for BT-1276. The presence of the primary amine was confirmed by an OPA assay and was found to contain 3.3 mol % primary amine.

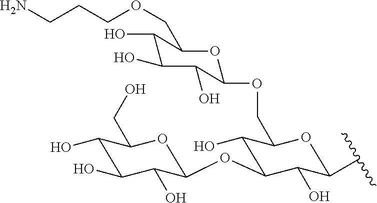 [SMILES: *C1OC(COC2OC(COCCCN)C(O)C(O)C2O)C(O)C(OC2OC(CO)C(O)C(O)C2O)C1O]

### Propyl Amine Laminarin Analog BT-1279

The procedure used to synthesize BT-1268 was repeated using laminarin (2 g) to afford 1.3 g of BT-1279. Elemental analysis by combustion gave a % N of 0.19%, which translates to 2.2 mol % propyl amine incorporation for BT-1279. The presence of the primary amine was confirmed by an OPA assay and was found to contain 2.6 mol % primary amine.

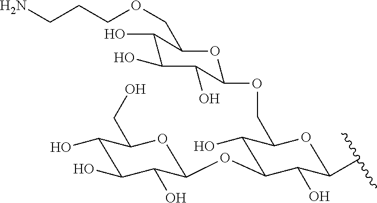 [SMILES: *C1OC(COC2OC(COCCCN)C(O)C(O)C2O)C(O)C(OC2OC(CO)C(O)C(O)C2O)C1O]

### Propyl Amine Scleroglucan Analog BT-1289

The procedure used to synthesize BT-1268 was repeated using solubilized scleroglucan (300 mg), see above for the preparation of soluble scleroglucan within the procedure for making Oxidized Scleroglucan 4, to afford 220 mg of BT-1289. Elemental analysis by combustion gave a % N of 0.19%, which translates to 2.2 mol % propyl amine incorporation for BT-1289. The presence of the primary amine was confirmed by an OPA assay and was found to contain 3.2 mol % primary amine.

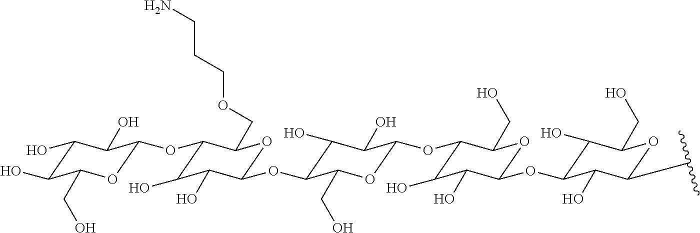 [SMILES: *C1OC(CO)C(O)C(OC2OC(CO)C(OC3OC(CO)C(OC4OC(COCCCN)C(OC5OC(CO)C(O)C(O)C5O)C(O)C4O)C(O)C3O)C(O)C2O)C1O]

### Propyl Amine Barley Glucan Analog BT-1292

The procedure used to synthesize BT-1268 was repeated using barley glucan to afford BT-1292. The presence of the primary amine was measured by an OPA assay and was found to contain 3.7 mol % primary amine.

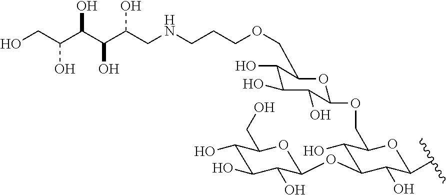 [SMILES: *C1OC(COC2OC(COCCCNC[C@@H](O)[C@@H](O)[C@H](O)[C@H](O)CO)C(O)C(O)C2O)C(O)C(OC2OC(CO)C(O)C(O)C2O)C1O]

### Mannose Propyl Amine MMW Yeast Glucan Analog BT-1243

To a solution of propyl amine MMW yeast glucan analog BT-1268 (60 mg) in sterile water (2.3 mL) and methanol (1.5 mL) was added mannose (33 mg) followed by borane pyridine complex (0.12 mL, 8.0 M in THF). The resulting solution was vortexed and placed at 50° C. for three days. After cooling to rt, the product was precipitated from the reaction mixture with ice cold ethanol and recovered by centrifugation. The pellet was washed with additional ethanol and dialyzed into sterile water over 3K centrifugal filtration devices to afford 59 mg of BT-1243. The loss of primary amine and proof of reductive amination was confirmed by an OPA assay. BT-1243 contained less than 0.1 mol % primary amine, indicating a complete conversion from the 3.1 mol % primary amine present in the starting material, BT-1268.

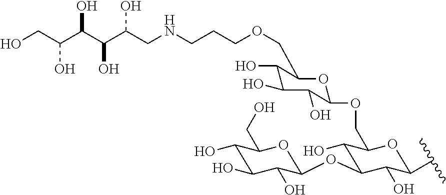 [SMILES: *C1OC(COC2OC(COCCCNC[C@@H](O)[C@@H](O)[C@H](O)[C@H](O)CO)C(O)C(O)C2O)C(O)C(OC2OC(CO)C(O)C(O)C2O)C1O]

### Galactose Propyl Amine MMW Yeast Glucan Analog BT-1245

The procedure used to synthesize BT-1243 was repeated using galactose (33 mg) and propyl amine MMW yeast glucan analog BT-1268 (60 mg) to afford 47 mg of BT-1245. The loss of primary amine and proof of reductive amination was confirmed by an OPA assay. BT-1245 contained 0.7 mol % primary amine, indicating a 77% conversion from the 3.1 mol % primary amine present in the starting material, BT-1268.

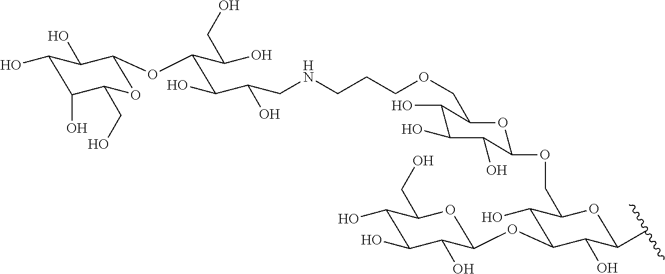 [SMILES: *C1OC(COC2OC(COCCCNCC(O)C(O)C(OC3OC(CO)C(O)C(O)C3O)C(O)CO)C(O)C(O)C2O)C(O)C(OC2OC(CO)C(O)C(O)C2O)C1O]

### Lactose Propyl Amine MMW Yeast Glucan Analog BT-1244

The procedure used to synthesize BT-1243 was repeated using lactose (63 mg) and propyl amine MMW yeast glucan analog BT-1268 (60 mg) to afford 54 mg of BT-1244. The loss of primary amine and proof of reductive amination was confirmed by an OPA assay. BT-1244 contained 0.5 mol % primary amine, indicating an 84% conversion from the 3.1 mol % primary amine present in the starting material, BT-1268.

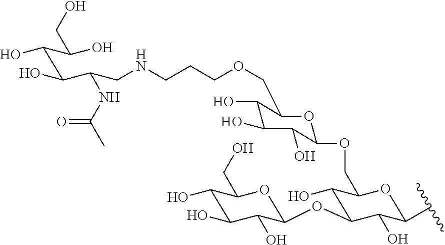 [SMILES: *C1OC(COC2OC(COCCCNCC(NC(C)=O)C(O)C(O)C(O)CO)C(O)C(O)C2O)C(O)C(OC2OC(CO)C(O)C(O)C2O)C1O]

### N-Acetyl Glucosamine Propyl Amine MMW Yeast Glucan Analog BT-1242

The procedure used to synthesize BT-1243 was repeated using N-acetyl glucosamine (41 mg) and propyl amine MMW yeast glucan analog BT-1268 (60 mg) to afford 26 mg of BT-1242. The loss of primary amine and proof of reductive amination was confirmed by an OPA assay. BT-1242 contained 1.1 mol % primary amine, indicating a 65% conversion from the 3.1 mol % primary amine present in the starting material, BT-1268.

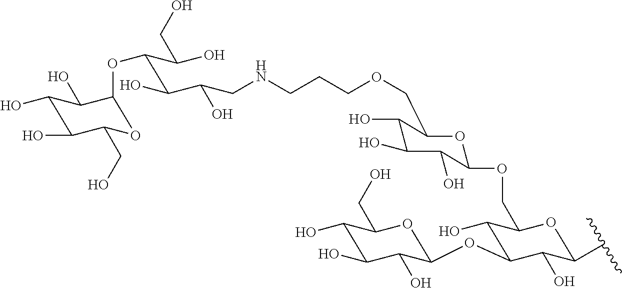 [SMILES: *C1OC(COC2OC(COCCCNCC(O)C(O)C(OC3OC(CO)C(O)C(O)C3O)C(O)CO)C(O)C(O)C2O)C(O)C(OC2OC(CO)C(O)C(O)C2O)C1O]

### Maltose Propyl Amine MMW Yeast Glucan Analog BT-1248

The procedure used to synthesize BT-1243 was repeated using maltose (63 mg) and propyl amine MMW yeast glucan analog BT-1268 (60 mg) to afford 60 mg of BT-1248. The loss of primary amine and proof of reductive amination was confirmed by an OPA assay. BT-1248 contained 0.3 mol % primary amine, indicating a 90% conversion from the 3.1 mol % primary amine present in the starting material, BT-1268.

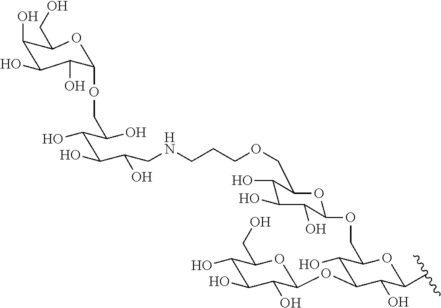 [SMILES: *C1OC(COC2OC(COCCCNCC(O)C(O)C(O)C(O)COC3OC(CO)C(O)C(O)C3O)C(O)C(O)C2O)C(O)C(OC2OC(CO)C(O)C(O)C2O)C1O]

### Melbiose Propyl Amine MMW Yeast Glucan Analog BT-1249

The procedure used to synthesize BT-1243 was repeated using melbiose (63 mg) and propyl amine MMW yeast glucan analog BT-1268 (60 mg) to afford 60 mg of BT-1249. The loss of primary amine and proof of reductive amination was confirmed by an OPA assay. BT-1249 contained 0.1 mol % primary amine, indicating a 97% conversion from the 3.1 mol % primary amine present in the starting material, BT-1268.

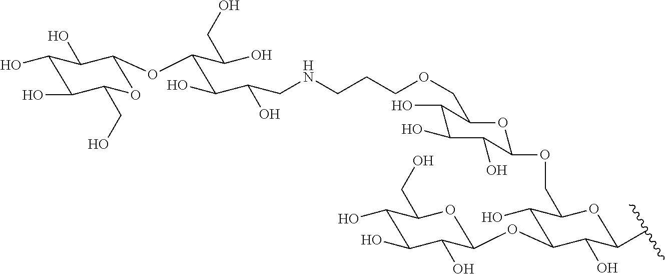 [SMILES: *C1OC(COC2OC(COCCCNCC(O)C(O)C(OC3OC(CO)C(O)C(O)C3O)C(O)CO)C(O)C(O)C2O)C(O)C(OC2OC(CO)C(O)C(O)C2O)C1O]

### Cellobiose Propyl Amine MMW Yeast Glucan Analog BT-1250

The procedure used to synthesize BT-1243 was repeated using cellobiose (63 mg) and propyl amine MMW yeast glucan analog BT-1268 (60 mg) to afford 60 mg of BT-1250. The loss of primary amine and proof of reductive amination was confirmed by an OPA assay. BT-1250 contained 0.2 mol % primary amine, indicating a 94% conversion from the 3.1 mol % primary amine present in the starting material, BT-1268.

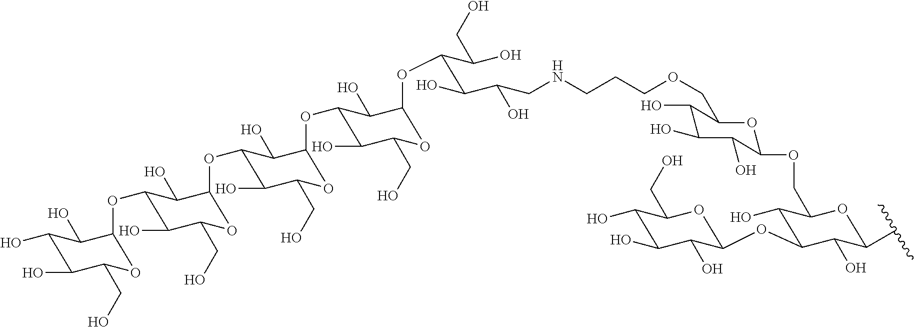 [SMILES: *C1OC(COC2OC(COCCCNCC(O)C(O)C(OC3OC(CO)C(O)C(OC4OC(CO)C(O)C(OC5OC(CO)C(O)C(OC6OC(CO)C(O)C(O)C6O)C5O)C4O)C3O)C(O)CO)C(O)C(O)C2O)C(O)C(OC2OC(CO)C(O)C(O)C2O)C1O]

### Maltopentaose Propyl Amine MMW Yeast Glucan Analog BT-1251

The procedure used to synthesize BT-1243 was repeated using maltopentaose (102 mg) and propyl amine MMW yeast glucan analog BT-1268 (40 mg) to afford 40 mg of BT-1251. The loss of primary amine and proof of reductive amination was confirmed by an OPA assay. BT-1251 contained 0.4 mol % primary amine, indicating a 87% conversion from the 3.1 mol % primary amine present in the starting material, BT-1268.

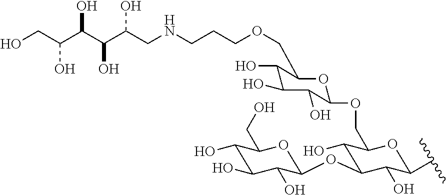 [SMILES: *C1OC(COC2OC(COCCCNC[C@@H](O)[C@@H](O)[C@H](O)[C@H](O)CO)C(O)C(O)C2O)C(O)C(OC2OC(CO)C(O)C(O)C2O)C1O]

### Mannose Propyl Amine LMW Yeast Glucan Analog BT-1278

The procedure used to synthesize BT-1243 was repeated using mannose (35 mg) and propyl amine LMW yeast glucan analog BT-1276 (50 mg) to afford 40 mg of BT-1278. The loss of primary amine and extent of reductive amination was confirmed by an OPA assay. BT-1278 contained less than 0.1 mol % primary amine, indicating a complete conversion from the 3.3 mol % primary amine present in the starting material, BT-1276.

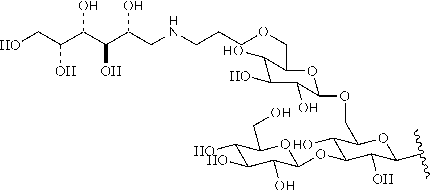 [SMILES: *C1OC(COC2OC(COCCCNC[C@@H](O)[C@@H](O)[C@@H](O)[C@H](O)CO)C(O)C(O)C2O)C(O)C(OC2OC(CO)C(O)C(O)C2O)C1O]

### Galactose Propyl Amine LMW Yeast Glucan Analog BT-1277

The procedure used to synthesize BT-1243 was repeated using galactose (35 mg) and propyl amine LMW yeast glucan analog BT-1276 (50 mg) to afford 42 mg of BT-1277. The loss of primary amine and extent of reductive amination was confirmed by an OPA assay. BT-1278 contained less than 0.1 mol % primary amine, indicating a complete conversion from the 3.3 mol % primary amine present in the starting material, BT-1276.

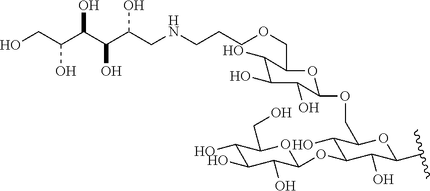 [SMILES: *C1OC(COC2OC(COCCCNC[C@@H](O)[C@@H](O)[C@H](O)[C@H](O)CO)C(O)C(O)C2O)C(O)C(OC2OC(CO)C(O)C(O)C2O)C1O]

### Mannose Propyl Amine Laminarin Analog BT-1281

The procedure used to synthesize BT-1243 was repeated using mannose (33 mg) and propyl amine laminarin analog BT-1279 (50 mg) to afford 41 mg of BT-1281. The loss of primary amine and proof of reductive amination was confirmed by an OPA assay. BT-1281 contained less than 0.1 mol % primary amine, indicating a complete conversion from the 2.6 mol % primary amine present in the starting material, BT-1279.

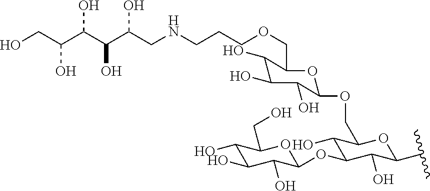 [SMILES: *C1OC(COC2OC(COCCCNC[C@@H](O)[C@@H](O)[C@@H](O)[C@H](O)CO)C(O)C(O)C2O)C(O)C(OC2OC(CO)C(O)C(O)C2O)C1O]

### Galactose Propyl Amine Laminarin Analog BT-1280

The procedure used to synthesize BT-1243 was repeated using galactose (33 mg) and propyl amine laminarin analog BT-1279 (50 mg) to afford 71 mg of BT-1280. The loss of primary amine and proof of reductive amination was confirmed by an OPA assay. BT-1280 contained less than 0.1 mol % primary amine, indicating a complete conversion from the 2.6 mol % primary amine present in the starting material, BT-1279.

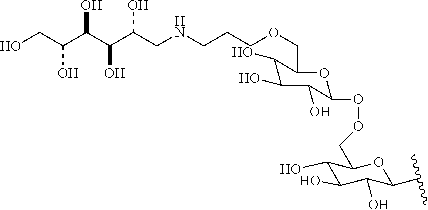 [SMILES: *C1OC(COOC2OC(COCCCNC[C@@H](O)[C@@H](O)[C@H](O)[C@H](O)CO)C(O)C(O)C2O)C(O)C(O)C1O]

### Mannose Propyl Amine Dextran Analog BT-1287

The procedure used to synthesize BT-1243 was repeated using mannose (16 mg) and propyl amine dextran analog BT-1266 (50 mg) to afford 43 mg of BT-1287. The loss of primary amine and proof of reductive amination was confirmed by an OPA assay. BT-1287 contained less than 0.1 mol % primary amine, indicating a complete conversion from the 2.6 mol % primary amine present in the starting material, BT-1266.

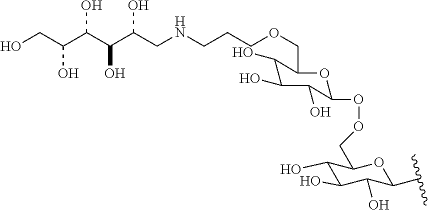 [SMILES: *C1OC(COOC2OC(COCCCNC[C@@H](O)[C@@H](O)[C@@H](O)[C@H](O)CO)C(O)C(O)C2O)C(O)C(O)C1O]

### Galactose Propyl Amine Dextran Analog BT-1286

The procedure used to synthesize BT-1243 was repeated using galactose (16 mg) and propyl amine dextran analog BT-1266 (50 mg) to afford 40 mg of BT-1286. The loss of primary amine and proof of reductive amination was confirmed by an OPA assay. BT-1286 contained less than 0.1 mol % primary amine, indicating a complete conversion from the 2.6 mol % primary amine present in the starting material, BT-1266.

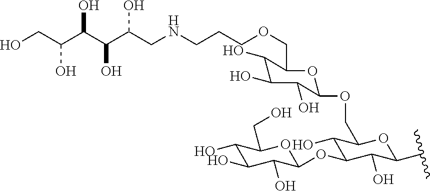 [SMILES: *C1OC(COC2OC(COCCCNC[C@@H](O)[C@@H](O)[C@H](O)[C@H](O)CO)C(O)C(O)C2O)C(O)C(OC2OC(CO)C(O)C(O)C2O)C1O]

### Mannose Propyl Amine Scleroglucan Analog BT-1290

The procedure used to synthesize BT-1243 was repeated using mannose (37 mg) and propyl amine scleroglucan analog BT-1289 (50 mg) to afford 36 mg of BT-1290. The loss of primary amine and proof of reductive amination was confirmed by an OPA assay. BT-1287 contained less than 0.1 mol % primary amine, indicating a complete conversion from the 3.4 mol % primary amine present in the starting material, BT-1289.

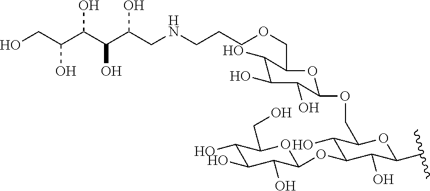 [SMILES: *C1OC(COC2OC(COCCCNC[C@@H](O)[C@@H](O)[C@@H](O)[C@H](O)CO)C(O)C(O)C2O)C(O)C(OC2OC(CO)C(O)C(O)C2O)C1O]

### Galactose Propyl Amine Scleroglucan Analog BT-1291

The procedure used to synthesize BT-1243 was repeated using galactose (37 mg) and propyl amine scleroglucan analog BT-1289 (50 mg) to afford 23 mg of BT-1291. The loss of primary amine and proof of reductive amination was confirmed by an OPA assay. BT-1287 contained less than 0.1 mol % primary amine, indicating a complete conversion from the 3.4 mol % primary amine present in the starting material, BT-1289.

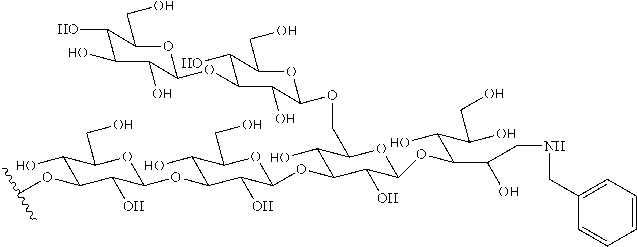 [SMILES: *OC1C(O)C(CO)OC(OC2C(O)C(CO)OC(OC3C(O)C(COC4OC(CO)C(O)C(OC5OC(CO)C(O)C(O)C5O)C4O)OC(OC(C(O)CNCc4ccccc4)C(O)C(O)CO)C3O)C2O)C1O]

### Benzyl Amine Reducing End MMW Yeast Glucan Analog BT-1275

To a 15 mL tube containing MMW yeast glucan (5 mL of a 10 mg/mL aqueous solution) was added benzyl amine (0.05 mL) and the reaction was swirled to mix, sealed, and placed in a 50° C. water bath for 18 hours. The reaction was cooled to rt and an aqueous solution of NaCNBH₃ (100 mg in 1.0 mL of dPBS) was added and the reaction was heated for an additional 18 hours at 50° C. The reaction mixture was combined with water and exhaustively dialyzed into sterile water using 3K centrifugal filtration units. The concentrated material was diluted into water and sterile filtered through a 0.2 μm syringe filter to afford an aqueous solution containing 31 mg of BT-1275. Elemental analysis by combustion on lyophilized material gave a % N of 0.23%. This translates to 2.7 mol % of benzyl amine with respect to the individual glucose monomers of the glucan.

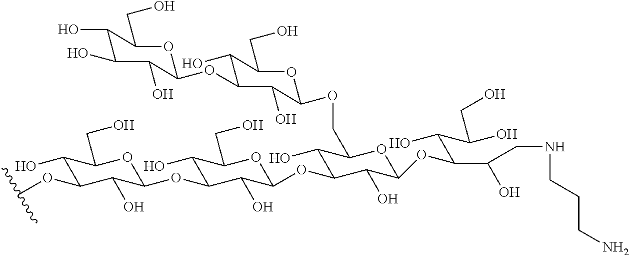 [SMILES: *OC1C(O)C(CO)OC(OC2C(O)C(CO)OC(OC3C(O)C(COC4OC(CO)C(O)C(OC5OC(CO)C(O)C(O)C5O)C4O)OC(OC(C(O)CNCCCN)C(O)C(O)CO)C3O)C2O)C1O]

### 1,3-Diaminopropane Reducing End MMW Yeast Glucan Analog BT-1294

To a 50 mL tube containing MMW yeast glucan (30 mg) in dPBS (10 mL) was added 1,3-diaminopropane (5 mL) and NaCNBH₃ (190 mg). The resulting reactions mixture was swirled to mix, sealed, and placed in a 50° C. water bath for 6 days. The product was precipitated with cold ethanol and collected by centrifugation. The pellet was washed with additional ethanol and the product was dialyzed using 3K centrifugal filtration units. The concentrated material was diluted into water and sterile filtered through a 0.2 μm syringe filter to afford an aqueous solution containing 12 mg of BT-1294. The presence of the primary amine was measured by an OPA assay and the compound was found to contain 2.3 mol % primary amine.

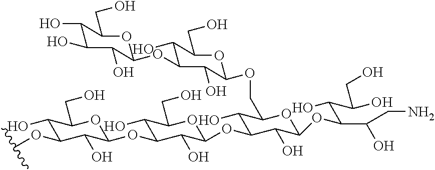 [SMILES: *OC1C(O)C(CO)OC(OC2C(O)C(CO)OC(OC3C(O)C(COC4OC(CO)C(O)C(OC5OC(CO)C(O)C(O)C5O)C4O)OC(OC(C(O)CN)C(O)C(O)CO)C3O)C2O)C1O]

### Ammonium Acetate Reducing End MMW Yeast Glucan Analog BT-1288

To a 125 mL bottle containing MMW yeast glucan (200 mg) in sterile water (100 mL) was added ammonium acetate (30 g) and NaCNBH₃ (1.3 g). The resulting reactions mixture was swirled to mix, sealed, and placed in a 50° C. water bath for 6 days. The product was precipitated with cold ethanol and collected by centrifugation. The pellet was washed with additional ethanol and the product was dialyzed using 3K centrifugal filtration units. The concentrated material was diluted into water and sterile filtered through a 0.2 μm syringe filter to afford an aqueous solution containing 161 mg of BT-1288. The presence of the primary amine was measured by an OPA assay and the compound was found to contain 2.0 mol % primary amine.

### De-Branched Scleroglucan Analogs BT-1322 and BT-1323

To 2 individual solutions of solubilized scleroglucan (120 mg), each in 10 mL sterile water, see above for the preparation of soluble scleroglucan within the procedure for making Oxidized Scleroglucan 4, was added NaIO₄ (61 mg) in 26 mL sterile water for reaction A (BT-1322) and (41 mg) in 26 mL sterile water for reaction B (BT-1323). Both were sealed and placed in the dark for 2 d. The reactions were quenched with ethylene glycol (0.03 mL) and desalted over 10K Pellicon tangential flow filtration devices. To solution A (60 mL volume) was added NH₄OH (2.4 mL, 5 N) and to solution B (70 mL volume) was added NH₄OH (2.8 mL, 5 N). NaBH₄ (0.25 g) was then added to each and the resulting solutions were loosely capped and held at room temperature overnight. Both reactions were quenched with glacial AcOH and dialyzed into sterile water over 10K Pellicon tangential flow filtration devices. To the resulting solution of reaction A (40 mL reaction volume) was added trifluoroacetic acid (TFA) (0.31 mL) and to reaction B (55 mL reaction volume) was added TFA (0.42 mL). The resulting solutions were held at 40° C. overnight and then dialyzed into sterile water over 10K Pellicon tangential flow filtration devices to afford 30 mg BT-1322 and 48 mg of BT-1323. Linkage analysis showed that the branching frequency was reduced from 33% for the soluble scleroglucan starting material to 12% for BT-1322 and to 17% for BT-1323. GPC/MALS analysis was used to prove that the reduction in the frequency of branching caused the formation of higher order aggregates and therefore higher molecular weights. The Mw values were increased from 89 kD for the starting material to 303 kD for BT-1322 and to 162 kD for BT-1323.

### Reduced [O] MMW Yeast Glucan Analog/SM BT-1156

To MMW Yeast Glucan (100 mg in 8.8 mL sterile water) was added NaIO₄ (24 mg in 1.2 mL water) and the reaction was placed in the dark and held over night at room temperature. To the resulting solution was added ethylene glycol (0.1 mL) and the resulting solution was allowed to stand at room temperature for 1 h at which point the solution was cooled to 0° C. and NaBH₄ (500 mg in 5 mL water) was added. The reaction was allowed to stand at room temperature over night. The reaction was dialyzed into sterile PBS over 3K centrifugal filtration devices and sterile 0.2 μm filtered to afford a solution containing 91 mg of BT-1156.

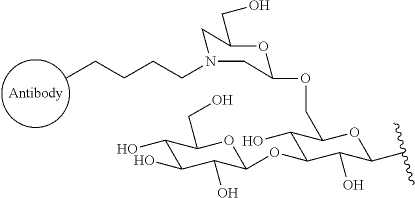 [SMILES: *C1OC(COC2CN(CCCCC)CC(CO)O2)C(O)C(OC2OC(CO)C(O)C(O)C2O)C1O]

### Erbitux MMW Yeast Glucan Analog BT-1200

To [O] MMW yeast glucan 1-H (50 mg) and sterile water (5 mL) was added Erbitux (25 mg in 12.5 mL 0.15 M aqueous NaCl). The reaction was swirled and placed in a 40° C. water bath for 24 hours. The reaction was removed from the water bath and cooled to 0° C. To the resulting solution was added NaCNBH₃ (100 mg in 1.0 mL PBS) and the reaction was swirled to mix, sealed, and placed in a 40° C. water bath for 24 hours. After cooling to room temperature the reaction mixture was dialyzed into sterile PBS using 10K centrifugal filtration units. The concentrated material was diluted into PBS and sterile filtered through a 0.2 μm syringe filter to afford 37 mg of BT-1215. The ratio of glucan to protein was 1.5:1, which was calculated by anthrone and the absorbance at 280 nm.

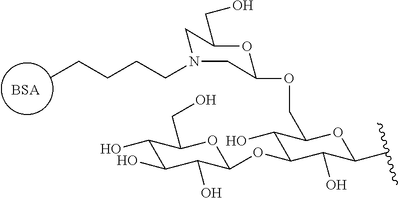 [SMILES: "BSA" ; *C1OC(COC2CN(CCCCC)CC(CO)O2)C(O)C(OC2OC(CO)C(O)C(O)C2O)C1O]

### Bovine Serum Albumin (BSA) MMW Yeast Glucan Analog BT-1215

To six 50 mL sterile centrifuge tubes each containing [O] MMW yeast glucan 1-H (10 mL of a 10 mg/mL aqueous solution) were added BSA (75 mg in 3.0 mL PBS). The reactions were swirled and placed in a 40° C. water bath for 24 hours. The reactions were removed from the water bath and cooled to 0° C. To the resulting solutions were added NaCNBH₃ (200 mg in 2.0 mL PBS) and the reactions were swirled to mix, sealed, and placed in a 40° C. water bath for 24 hours. After cooling to room temperature the reaction mixtures were next dialyzed into sterile PBS using 100K centrifugal filtration units. The concentrated material was diluted into PBS and sterile filtered through a 0.2 μm syringe filter to afford 750 mg of BT-1215. The ratio of glucan to protein was 1.2:1, which was calculated by subtracting the concentration of BSA, as determined by using the absorbance at 280 nm, from the overall concentration of the conjugate, as determined by integration of the GPC refractive index trace.

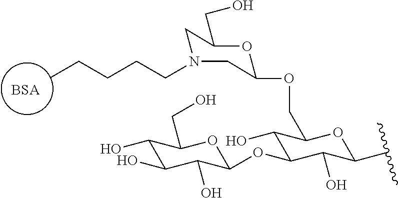 [SMILES: "BSA" ; *C1OC(COC2CN(CCCCC)CC(CO)O2)C(O)C(OC2OC(CO)C(O)C(O)C2O)C1O]

### Bovine Serum Albumin (BSA) Whole Glucan Particle (WGP) Yeast Glucan Analog BT-1213

### [O] WGP Yeast Glucan 5

To a thoroughly sonicated aqueous solution of insoluble WGP yeast glucan of known concentration was added NaIO₄ to obtain a ratio of 0.3 mg of NaIO₄ per mg of glucan. The final reaction concentration, with respect to glucan, was adjusted to 10 mg/mL with sterile water. The reaction was swirled to mix and placed in the dark for 20 hours. The oxidized product was collected and washed with sterile PBS by centrifugation.

### Reductive Amination of [O] WGP Yeast Glucan 5 with BSA

To [O] WGP Yeast Glucan 5 (1.5 g) in 150 mL PBS was added BSA (3.0 g). The reaction was swirled and placed in a 40° C. water bath for 24 hours. The reaction was removed from the water bath and cooled to 0° C. To the resulting solution was added NaCNBH₃ (3.0 g in 30.0 mL PBS) and the reaction was swirled to mix, sealed, and placed in a 40° C. water bath for 24 hours. After cooling to room temperature the mixture was centrifuged and washed with sterile PBS to afford 1.2 g of BT-1213. The ratio of glucan to protein was 1.7:1, which was calculated by measuring the amount of BSA, as determined by elemental analysis, and the concentration of glucan, as determined by the anthrone assay.

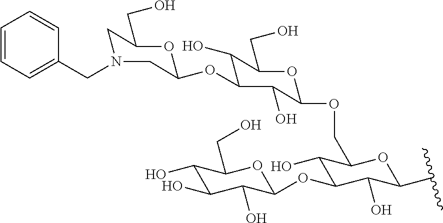 [SMILES: *C1OC(COC2OC(CO)C(O)C(OC3CN(Cc4ccccc4)CC(CO)O3)C2O)C(O)C(OC2OC(CO)C(O)C(O)C2O)C1O]

### Benzyl Amine WGP Yeast Glucan Analog BT-1357

To [O] WGP Yeast Glucan 5 (600 mg) in sterile water (60 mL) was added benzylamine (1.2 mL). The reaction was swirled and placed in a 50° C. water bath for 24 hours. The reaction was removed from the water bath and cooled to 0° C. To the resulting solution was added NaCNBH₃ (1.2 g in 12.0 mL PBS) and the reaction was swirled to mix, sealed, and placed in a 50° C. water bath for 24 hours. After cooling to room temperature the mixture was centrifuged and washed with sterile water to afford 310 mg of BT-1357. Elemental analysis by combustion on lyophilized material gave a % N of 2.14%. This translates to 27 mol % of benzyl amine with respect to the individual glucose monomers of the glucan.

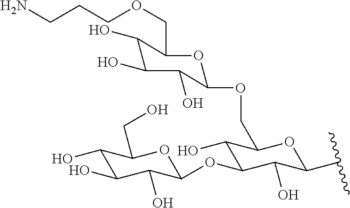 [SMILES: *C1OC(COC2OC(COCCCN)C(O)C(O)C2O)C(O)C(OC2OC(CO)C(O)C(O)C2O)C1O]

### Propyl Amine MMW Yeast Glucan Analog BT-1379

The procedure used to synthesize BT-1268 was repeated to afford BT-1379. Elemental analysis by combustion gave a % N of 0.32%, which translates to 3.8 mol % propyl amine incorporation for BT-1379. The presence of the primary amine was confirmed by an OPA assay and was found to contain 1.74 mol % primary amine.

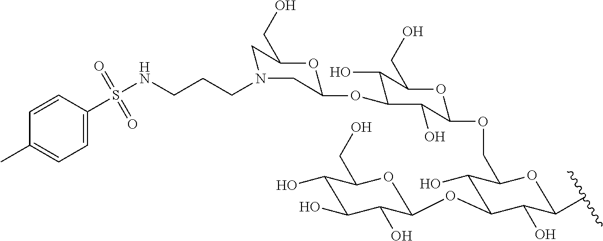 [SMILES: *C1OC(COC2OC(CO)C(O)C(OC3CN(CCCNS(=O)(=O)c4ccc(C)cc4)CC(CO)O3)C2O)C(O)C(OC2OC(CO)C(O)C(O)C2O)C1O]

### Toluene Sulfonamide MMW Yeast Glucan Analog BT-1381

To a solution of diaminopropane MMW yeast glucan analog BT-1253 (50 mg) in sterile water (2.9 mL) was added aqueous sodium carbonate (0.67 mL, 0.04 M) and sterile water (1.4 mL). To the resulting solution was added p-toluene sulfonyl chloride (2.6 mg dissolved in acetone (0.26 mL)) dropwise. The solution was rotated end over end for 1 hour and placed at 4° C. overnight. The solution was dialyzed into sterile water over 10K centrifugal filtration devices to afford 38 mg of BT-1381. The loss of primary amine and sulfonamide formation was an 80% conversion from the sm, BT-1253. The retention of nitrogen was confirmed by combustion analysis, giving a % N of 0.54% for BT-1381 compared to 0.69% for the sm, BT-1253.

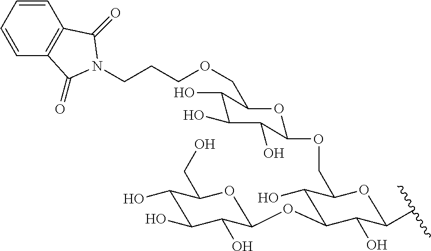 [SMILES: *C1OC(COC2OC(COCCCN3C(=O)c4ccccc4C3=O)C(O)C(O)C2O)C(O)C(OC2OC(CO)C(O)C(O)C2O)C1O]

### Propyl Phthalimide MMW Yeast Glucan Analog BT-1382

The procedure used to synthesize BT-1268 was repeated using MMW yeast glucan (100 mg), with the exceptions of using N-(3-bromopropyl)phthalimide in place of 1-bromo-3-aminopropane hydrobromide and dialyzing directly without precipitation, to afford 29 mg of BT-1382. Elemental analysis by combustion gave a % N of 0.13%, which translates to 1.5 mol % propyl amine incorporation for BT-1382.

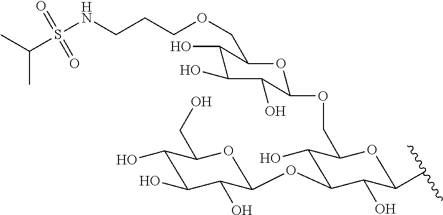 [SMILES: *C1OC(COC2OC(COCCCNS(=O)(=O)C(C)C)C(O)C(O)C2O)C(O)C(OC2OC(CO)C(O)C(O)C2O)C1O]

### 2-Propane Sulfonamide MMW Yeast Glucan Analog BT-1397

To a solution of propyl amine MMW yeast glucan analog BT-1379 (50 mg) in sterile water (1.85 mL) was added aqueous sodium carbonate (0.04 M, 0.34 mL) and sterile water (2.8 mL). To the resulting solution was added 2-propanesulfonyl chloride (1.2 μL dissolved in acetone (0.12 mL)) dropwise. The solution was rotated end over end for 1 hour and placed at 4° C. overnight. The solution was dialyzed into sterile water over 10K centrifugal filtration devices to afford 35 mg of BT-1397. The loss of primary amine and sulfonamide formation was confirmed by an OPA assay. BT-1397 contained 1.53 mol % primary amine, indicating a 12% incorporation from the 1.74 mol % primary amine present in the sm, BT-1379. The retention of nitrogen was confirmed by combustion analysis, giving a % N of 0.34% for BT-1397 compared to 0.32% for the sm, BT-1379.

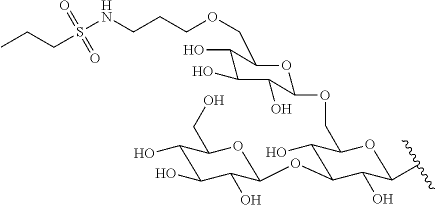 [SMILES: *C1OC(COC2OC(COCCCNS(=O)(=O)CCC)C(O)C(O)C2O)C(O)C(OC2OC(CO)C(O)C(O)C2O)C1O]

### 1-Propane Sulfonamide MMW Yeast Glucan Analog BT-1398

The procedure used to synthesize BT-1397 was repeated using 1-propane sulfonyl chloride (1.2 μL) and propyl amine MMW yeast glucan analog BT-1379 (50 mg) to afford 40 mg of BT-1398. The loss of primary amine and sulfonamide formation was confirmed by an OPA assay. BT-1398 contained 1.46 mol % primary amine, indicating a 16% incorporation from the 1.74 mol % primary amine present in the starting material, BT-1379. The retention of nitrogen was confirmed by combustion analysis, giving a % N of 0.32% for BT-1398 compared to 0.32% for the sm, BT-1379.

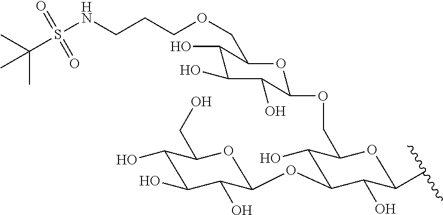 [SMILES: *C1OC(COC2OC(COCCCNS(=O)(=O)C(C)(C)C)C(O)C(O)C2O)C(O)C(OC2OC(CO)C(O)C(O)C2O)C1O]

### Isobutyl Sulfonamide MMW Yeast Glucan Analog BT-1399

The procedure used to synthesize BT-1397 was repeated using isobutyl sulfonyl chloride (1.3 μL) and propyl amine MMW yeast glucan analog BT-1379 (50 mg) to afford 40 mg of BT-1399. The loss of primary amine and sulfonamide formation was confirmed by an OPA assay. BT-1399 contained 1.44 mol % primary amine, indicating a 17% incorporation from the 1.74 mol % primary amine present in the starting material, BT-1379. The retention of nitrogen was confirmed by combustion analysis, giving a % N of 0.28% for BT-1399 compared to 0.32% for the sm, BT-1379.

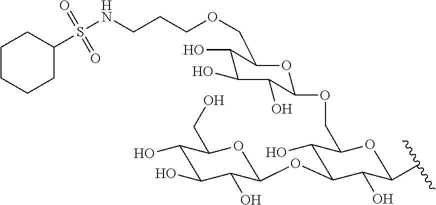 [SMILES: *C1OC(COC2OC(COCCCNS(=O)(=O)C3CCCCC3)C(O)C(O)C2O)C(O)C(OC2OC(CO)C(O)C(O)C2O)C1O]

### Cyclohexyl Sulfonamide MMW Yeast Glucan Analog BT-1400

The procedure used to synthesize BT-1397 was repeated using cyclohexyl sulfonyl chloride (1.5 μL) and propyl amine MMW yeast glucan analog BT-1379 (50 mg) to afford 42 mg of BT-1400. The loss of primary amine and sulfonamide formation was confirmed by an OPA assay. BT-1400 contained 1.59 mol % primary amine, indicating a 8.6% incorporation from the 1.74 mol % primary amine present in the starting material, BT-1379. The retention of nitrogen was confirmed by combustion analysis, giving a % N of 0.19% for BT-1400 compared to 0.32% for the sm, BT-1379.

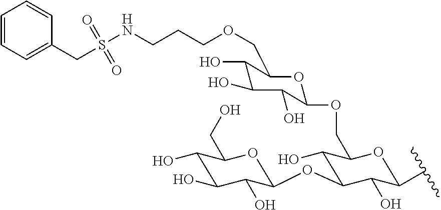 [SMILES: *C1OC(COC2OC(COCCCNS(=O)(=O)Cc3ccccc3)C(O)C(O)C2O)C(O)C(OC2OC(CO)C(O)C(O)C2O)C1O]

### Phenylmethyl Sulfonamide MMW Yeast Glucan Analog BT-1401

The procedure used to synthesize BT-1397 was repeated using Isobutyl sulfonyl chloride (1.5 μL) and propyl amine MMW yeast glucan analog BT-1379 (50 mg) to afford 39 mg of BT-1401. The loss of primary amine and sulfonamide formation was confirmed by an OPA assay. BT-1401 contained 1.59 mol % primary amine, indicating a 8.6% incorporation from the 1.74 mol % primary amine present in the starting material, BT-1379. The retention of nitrogen was confirmed by combustion analysis, giving a % N of 0.26% for BT-1401 compared to 0.32% for the sm, BT-1379.

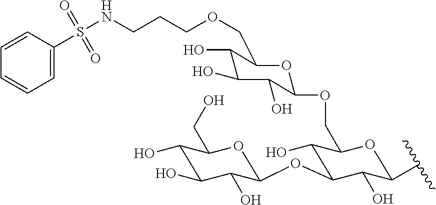 [SMILES: *C1OC(COC2OC(COCCCNS(=O)(=O)c3ccccc3)C(O)C(O)C2O)C(O)C(OC2OC(CO)C(O)C(O)C2O)C1O]

### Phenyl Sulfonamide MMW Yeast Glucan Analog BT-1402

The procedure used to synthesize BT-1397 was repeated using phenyl sulfonyl chloride (1.4 μL) and propyl amine MMW yeast glucan analog BT-1379 (50 mg) to afford 40 mg of BT-1402. The loss of primary amine and sulfonamide formation was confirmed by an OPA assay. BT-1402 contained 0.86 mol % primary amine, indicating a 51% incorporation from the 1.74 mol % primary amine present in the starting material, BT-1379. The retention of nitrogen was confirmed by combustion analysis, giving a % N of 0.16% for BT-1402 compared to 0.32% for the sm, BT-1379.

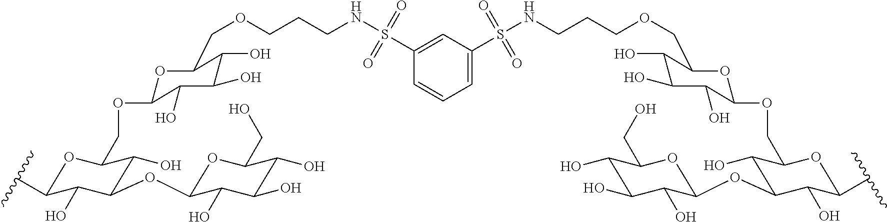 [SMILES: *C1OC(COC2OC(COCCCNS(=O)(=O)c3cccc(S(=O)(=O)NCCCOCC4OC(OCC5OC(*)C(O)C(OC6OC(CO)C(O)C(O)C6O)C5O)C(O)C(O)C4O)c3)C(O)C(O)C2O)C(O)C(OC2OC(CO)C(O)C(O)C2O)C1O]

### 1,3-Phenyl Bis-Sulfonamide MMW Yeast Glucan Analog BT-1403

The procedure used to synthesize BT-1397 was repeated using 1,3-phenyl bis sulfonyl chloride (2.2 μL) and propyl amine MMW yeast glucan analog BT-1379 (50 mg) to afford 43 mg of BT-1403. The loss of primary amine and sulfonamide formation was confirmed by an OPA assay. BT-1403 contained 0.92 mol % primary amine, indicating a 47% incorporation from the 1.74 mol % primary amine present in the starting material, BT-1379. The retention of nitrogen was confirmed by combustion analysis, giving a % N of 0.25% for BT-1403 compared to 0.32% for the sm, BT-1379.

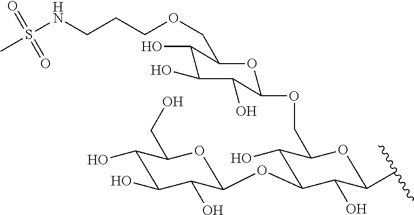 [SMILES: *C1OC(COC2OC(COCCCNS(C)(=O)=O)C(O)C(O)C2O)C(O)C(OC2OC(CO)C(O)C(O)C2O)C1O]

### Methane Sulfonamide MMW Yeast Glucan Analog BT-1404

The procedure used to synthesize BT-1397 was repeated using methane sulfonyl chloride (1.5 μL) and propyl amine MMW yeast glucan analog BT-1379 (50 mg) to afford 40 mg of BT-1404. The loss of primary amine and sulfonamide formation was confirmed by an OPA assay. BT-1404 contained 1.53 mol % primary amine, indicating a 12% incorporation from the 1.74 mol % primary amine present in the starting material, BT-1379. The retention of nitrogen was confirmed by combustion analysis, giving a % N of 0.25% for BT-1404 compared to 0.32% for the sm, BT-1379.

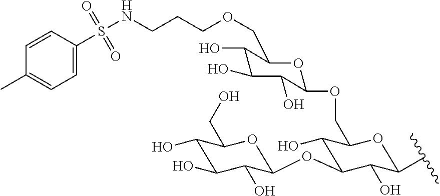 [SMILES: *C1OC(COC2OC(COCCCNS(=O)(=O)c3ccc(C)cc3)C(O)C(O)C2O)C(O)C(OC2OC(CO)C(O)C(O)C2O)C1O]

### p-Toluene Sulfonamide MMW Yeast Glucan Analog BT-1405

The procedure used to synthesize BT-1397 was repeated using p-toluene sulfonyl chloride (2.6 μL) and propyl amine MMW yeast glucan analog BT-1379 (50 mg) to afford 40 mg of BT-1405. The loss of primary amine and sulfonamide formation was confirmed by an OPA assay. BT-1405 contained 1.02 mol % primary amine, indicating a 41% incorporation from the 1.74 mol % primary amine present in the starting material, BT-1379. The retention of nitrogen was confirmed by combustion analysis, giving a % N of 0.22% for BT-1405 compared to 0.32% for the sm, BT-1379.

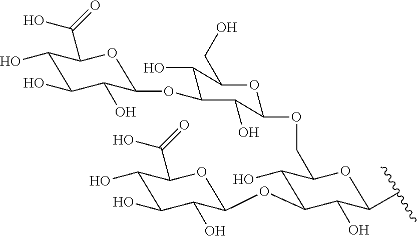 [SMILES: *C1OC(COC2OC(CO)C(O)C(OC3OC(C(=O)O)C(O)C(O)C3O)C2O)C(O)C(OC2OC(C(=O)O)C(O)C(O)C2O)C1O]

### TEMPO oxidized MMW Yeast Glucan Analog BT-1407

To MMW Yeast Glucan (200 mg) in sterile water (20 mL) was added (2,2,6,6-Tetramethylpiperidin-1-yl)oxyl (3.6 mg) and sodium bromide (20 mg). While the solution was stirring, aqueous sodium hydroxide (0.1 mL, 2M) was added to raise the pH above 11. Then aqueous sodium hypochlorite (0.72 mL, 12.5%) was added to the mixing solution. Once the solution had a stable pH of 11, sodium borohydride (20 mg) was added. The resulting solution was neutralized using aqueous HCl (4M) and then dialyzed into sterile water over 10K centrifugal filtration devices and sterile 0.2 μm filtered to afford a solution containing 135 mg of BT-1407 with 11 mol % glucoronic acid. The amount of oxidation was determined by first converting a sample of the acid to the nitrophenyl hydrazide and then detecting by fluorescence. (Analytical Letters, 1982, 15(A20) 1629-1642). This procedure was repeated with a second sample of MMW Yeast Glucan (1 g) to afford an additional sample of BT-1407 with 5.8 mol % glucoronic acid.

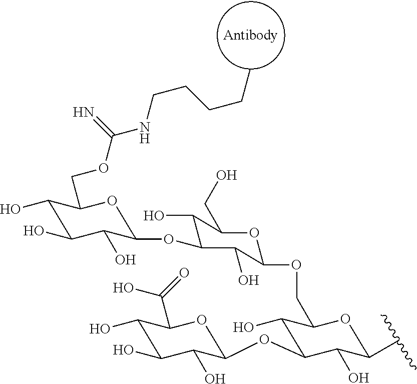 [SMILES: *C1OC(COC2OC(CO)C(O)C(OC3OC(COC(=N)NCCCCC)C(O)C(O)C3O)C2O)C(O)C(OC2OC(C(=O)O)C(O)C(O)C2O)C1O]

### Erbitux MMW Yeast Glucan Analog BT-1409

To a stirring solution of MMW Yeast Glucan (25 mg) in sterile water (2.5 mL) was added 1-cyano-4-dimethylaminopyridinium tetrafluoroborate (CDAP) (0.333 mL of 100 mg/mL CDAP in acetonitrile). After 1 minute of stirring, 0.3 M triethylamine in water (2.5 mL) was added to get the pH above 9. The resulting solution was added to a stirring solution of Erbitux (12.5 mL, 2 mg/mL in citrate saline). Ethanolamine (1 mL, 1M aqueous) was added and the solution was held overnight at 4° C. The solution was dialyzed into sterile PBS using a 10K tangential flow filtration device and sterile 0.2 μm filtered to afford a solution containing 14 mg of BT-1409. A 1:1.3 ratio of glucan to protein was determined by anthrone and absorbance at 280 nm.

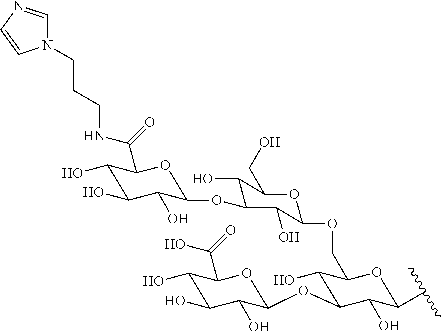 [SMILES: *C1OC(COC2OC(CO)C(O)C(OC3OC(C(=O)NCCCn4ccnc4)C(O)C(O)C3O)C2O)C(O)C(OC2OC(C(=O)O)C(O)C(O)C2O)C1O]

### Imidazole Propyl Amide MMW Yeast Glucan Analog BT-1512

To TEMPO oxidized MMW Yeast Glucan Analog BT-1407 (40 mg) and sterile water (4 mL) was added 1-(3 aminopropyl)-imidazole (0.36 mL), N-ethoxycarbonyl-2-ethoxy-1,2-dihydroquinoline (EEDQ) (180 mg), a stir bar, and 1M aqueous HCl (1 mL). The resulting solution was stirred overnight at rt at which point the solution was dialyzed into sterile water over a 10K centrifugal filtration device, and sterile 0.2 μm filtered to afford a solution containing 24 mg of BT-1512 that contained 5.3 weight % glucuronic acid as compared to the 5.8 weight % present in the sm, BT-1407, indicating an 8% conversion.

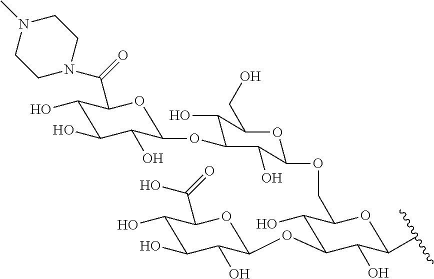 [SMILES: *C1OC(COC2OC(CO)C(O)C(OC3OC(C(=O)N4CCN(C)CC4)C(O)C(O)C3O)C2O)C(O)C(OC2OC(C(=O)O)C(O)C(O)C2O)C1O]

### 1-Methyl Piperazine Amide MMW Yeast Glucan Analog BT-1513

The procedure used to synthesize BT-1512 was repeated using 1-methyl piperazine (0.34 mL) and TEMPO oxidized MMW Yeast Glucan Analog BT-1407 (40 mg) to afford 23 mg of BT-1513 that contained 5.5 weight % glucuronic acid as compared to the 5.8 weight % present in the sm, BT-1407, indicating a 5% conversion.

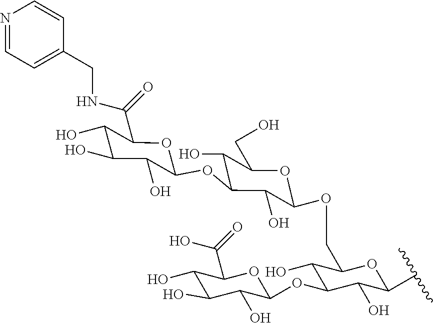 [SMILES: *C1OC(COC2OC(CO)C(O)C(OC3OC(C(=O)NCc4ccncc4)C(O)C(O)C3O)C2O)C(O)C(OC2OC(C(=O)O)C(O)C(O)C2O)C1O]

### 4-Methyl Pyridine Amide MMW Yeast Glucan Analog BT-1514

The procedure used to synthesize BT-1512 was repeated using 4-methyl pyridine (0.31 mL) and TEMPO oxidized MMW Yeast Glucan Analog BT-1407 (40 mg) to afford 23 mg of BT-1514 that contained 5.2 weight % glucuronic acid as compared to the 5.8 weight % present in the sm, BT-1407, indicating a 11% conversion.

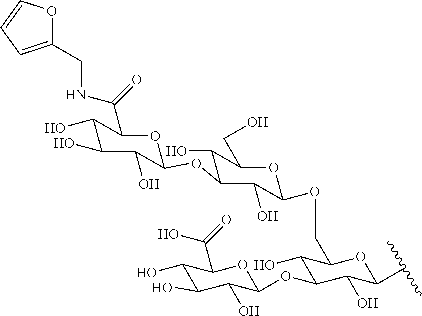 [SMILES: *C1OC(COC2OC(CO)C(O)C(OC3OC(C(=O)NCc4ccco4)C(O)C(O)C3O)C2O)C(O)C(OC2OC(C(=O)O)C(O)C(O)C2O)C1O]

### 2-Furfuryl Amide MMW Yeast Glucan Analog BT-1515

The procedure used to synthesize BT-1512 was repeated using 2-furfuryl amine (310 mg) and TEMPO oxidized MMW Yeast Glucan Analog BT-1407 (40 mg) to afford 25 mg of BT-1515 that contained 3.7 weight % glucuronic acid as compared to the 5.8 weight % present in the sm, BT-1407, indicating a 36% conversion.

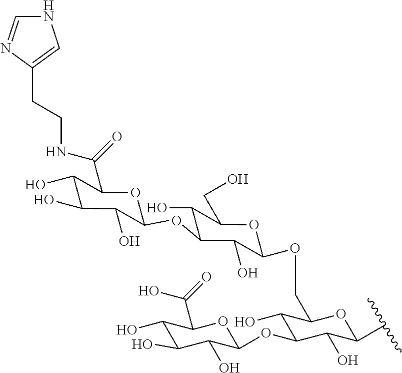 [SMILES: *C1OC(COC2OC(CO)C(O)C(OC3OC(C(=O)NCCc4c[nH]cn4)C(O)C(O)C3O)C2O)C(O)C(OC2OC(C(=O)O)C(O)C(O)C2O)C1O]

### Histamine Amide MMW Yeast Glucan Analog BT-1516

The procedure used to synthesize BT-1512 was repeated using histamine bis-HCl (560 mg) and TEMPO oxidized MMW Yeast Glucan Analog BT-1407 (40 mg) to afford 25 mg of BT-1516 that contained 5.1 weight % glucuronic acid as compared to the 5.8 weight % present in the sm, BT-1407, indicating a 11% conversion.

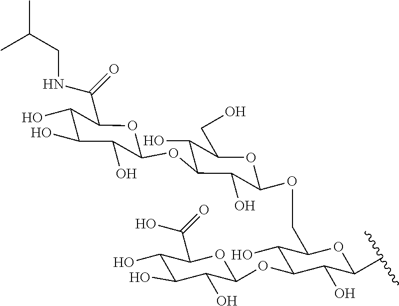 [SMILES: *C1OC(COC2OC(CO)C(O)C(OC3OC(C(=O)NCC(C)C)C(O)C(O)C3O)C2O)C(O)C(OC2OC(C(=O)O)C(O)C(O)C2O)C1O]

### Isobutyl Amide MMW Yeast Glucan Analog BT-1517

The procedure used to synthesize BT-1512 was repeated using isobutylamine HCl (330 mg) and TEMPO oxidized MMW Yeast Glucan Analog BT-1407 (40 mg) to afford 5 mg of BT-1517 that contained 5.2 weight % glucuronic acid as compared to the 5.8 weight % present in the sm, BT-1407, indicating a 10% conversion.

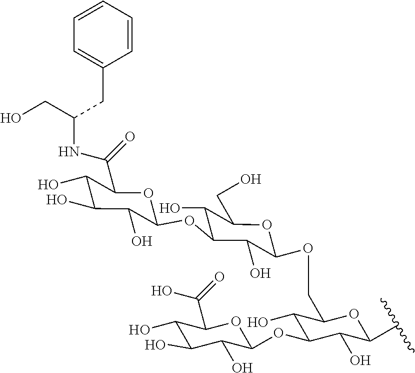 [SMILES: *C1OC(COC2OC(CO)C(O)C(OC3OC(C(=O)N[C@H](CO)Cc4ccccc4)C(O)C(O)C3O)C2O)C(O)C(OC2OC(C(=O)O)C(O)C(O)C2O)C1O]

### L-(−)-2-Amino-3-Phenyl-1-Propanol Amide MMW Yeast Glucan Analog BT-1518

The procedure used to synthesize BT-1512 was repeated using L-(−)-2-amino-3-phenyl-1-propanol (460 mg) and TEMPO oxidized MMW Yeast Glucan Analog BT-1407 (40 mg) to afford 22 mg of BT-1518 that contained 4.0 weight % glucuronic acid as compared to the 5.8 weight % present in the sm, BT-1407, indicating a 31% conversion.

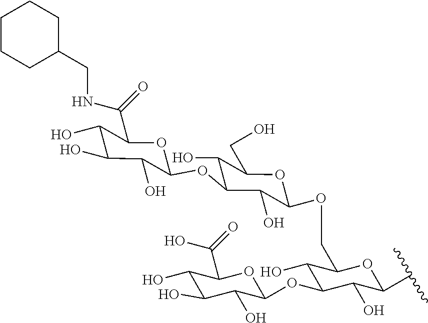 [SMILES: *C1OC(COC2OC(CO)C(O)C(OC3OC(C(=O)NCC4CCCCC4)C(O)C(O)C3O)C2O)C(O)C(OC2OC(C(=O)O)C(O)C(O)C2O)C1O]

### Cyclohexylmethyl Amide MMW Yeast Glucan Analog BT-1519

The procedure used to synthesize BT-1512 was repeated using cyclohexylmethylamine (0.39 mL) and TEMPO oxidized MMW Yeast Glucan Analog BT-1407 (40 mg) to afford 23 mg of BT-1519 that contained 3.8 weight % glucuronic acid as compared to the 5.8 weight % present in the sm, BT-1407, indicating a 34% conversion.

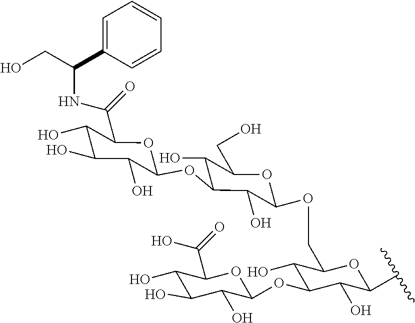 [SMILES: *C1OC(COC2OC(CO)C(O)C(OC3OC(C(=O)N[C@@H](CO)c4ccccc4)C(O)C(O)C3O)C2O)C(O)C(OC2OC(C(=O)O)C(O)C(O)C2O)C1O]

### R-(−)-2-Phenylglycinol Amide MMW Yeast Glucan Analog BT-1520

The procedure used to synthesize BT-1512 was repeated using R-(−)-2-phenylglycinol amine (420 mg) and TEMPO oxidized MMW Yeast Glucan Analog BT-1407 (40 mg) to afford 22 mg of BT-1520 that contained 3.6 weight % glucuronic acid as compared to the 5.8 weight % present in the sm, BT-1407, indicating a 38% conversion.

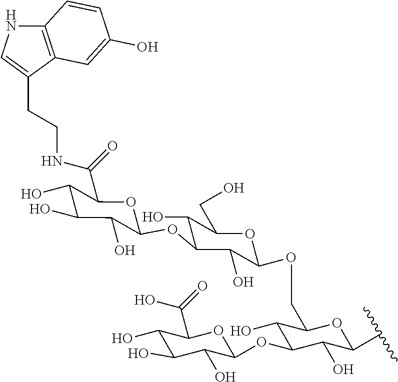 [SMILES: *C1OC(COC2OC(CO)C(O)C(OC3OC(C(=O)NCCc4c[nH]c5ccc(O)cc45)C(O)C(O)C3O)C2O)C(O)C(OC2OC(C(=O)O)C(O)C(O)C2O)C1O]

### Serotonin Amide MMW Yeast Glucan Analog BT-1521

The procedure used to synthesize BT-1512 was repeated using serotonin HCl (600 mg) and TEMPO oxidized MMW Yeast Glucan Analog BT-1407 (40 mg) to afford 28 mg of BT-1521 that contained 5.2 weight % glucuronic acid as compared to the 5.8 weight % present in the sm, BT-1407, indicating a 11% conversion.

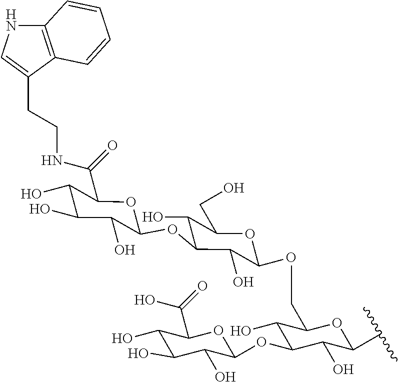 [SMILES: *C1OC(COC2OC(CO)C(O)C(OC3OC(C(=O)NCCc4c[nH]c5ccccc45)C(O)C(O)C3O)C2O)C(O)C(OC2OC(C(=O)O)C(O)C(O)C2O)C1O]

### Tryptamine Amide MMW Yeast Glucan Analog BT-1522

The procedure used to synthesize BT-1512 was repeated using tryptamine HCl (600 mg) and TEMPO oxidized MMW Yeast Glucan Analog BT-1407 (40 mg) to afford 28 mg of BT-1522 that contained 5.0 weight % glucuronic acid as compared to the 5.8 weight % present in the sm, BT-1407, indicating a 14% conversion.

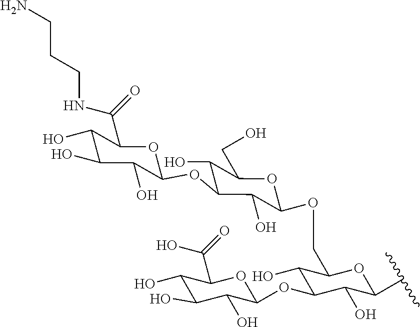 [SMILES: *C1OC(COC2OC(CO)C(O)C(OC3OC(C(=O)NCCCN)C(O)C(O)C3O)C2O)C(O)C(OC2OC(C(=O)O)C(O)C(O)C2O)C1O]

### Aminopropyl Amide MMW Yeast Glucan Analog BT-1523

The procedure used to synthesize BT-1512 was repeated using 1,3-diaminopropane (0.26 mL) and TEMPO oxidized MMW Yeast Glucan Analog BT-1407 (40 mg) to afford 8 mg of BT-1523 that contained 3.8 weight % glucuronic acid as compared to the 5.8 weight % present in the sm, BT-1407, indicating a 34% conversion.

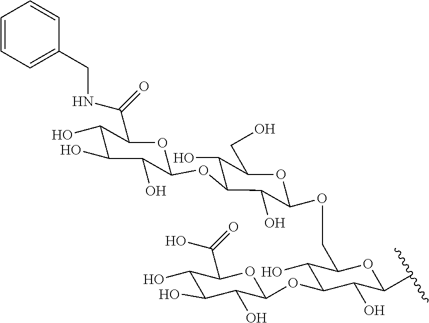 [SMILES: *C1OC(COC2OC(CO)C(O)C(OC3OC(C(=O)NCc4ccccc4)C(O)C(O)C3O)C2O)C(O)C(OC2OC(C(=O)O)C(O)C(O)C2O)C1O]

### Benzyl Amide MMW Yeast Glucan Analog BT-1628

The procedure used to synthesize BT-1512 was repeated using benzylamine (0.33 mL) and TEMPO oxidized MMW Yeast Glucan Analog BT-1407 (50 mg) to afford BT-1628 that contained 2.9 weight % glucuronic acid as compared to the 5.8 weight % present in the sm, BT-1407, indicating a 50% conversion.

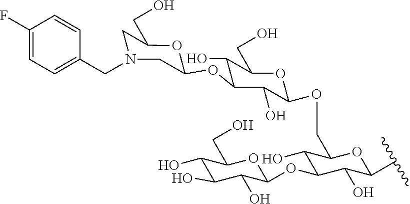 [SMILES: *C1OC(COC2OC(CO)C(O)C(OC3CN(Cc4ccc(F)cc4)CC(CO)O3)C2O)C(O)C(OC2OC(CO)C(O)C(O)C2O)C1O]

### 4-F-Benzylamine MMW Yeast Glucan Analog BT-1527

The procedure used to synthesize BT-1222 was repeated using 4-fluorobenzylamine (50 mg) and [O] MMW yeast glucan 1-L (50 mg), with the exception that both heating steps were performed at 50° C. to afford 19 mg of BT-1527. Elemental analysis by combustion gave a % N of 0.27%, which translates to 3.2 mol % 4-fluorobenzylamine incorporation.

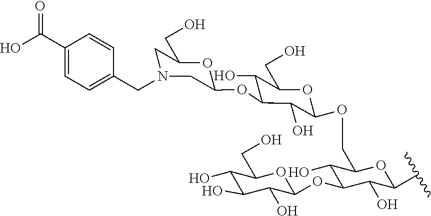 [SMILES: *C1OC(COC2OC(CO)C(O)C(OC3CN(Cc4ccc(C(=O)O)cc4)CC(CO)O3)C2O)C(O)C(OC2OC(CO)C(O)C(O)C2O)C1O]

### 4-Aminomethyl Benzoic Acid MMW Yeast Glucan Analog BT-1528

The procedure used to synthesize BT-1222 was repeated using 4-aminomethyl benzoic acid (50 mg) and [O] MMW yeast glucan 1-L (50 mg), with the exception that both heating steps were performed at 50° C., to afford 38 mg of BT-1528. Elemental analysis by combustion gave a % N of 0.25%, which translates to 2.9 mol % 4-aminomethyl benzoic acid incorporation.

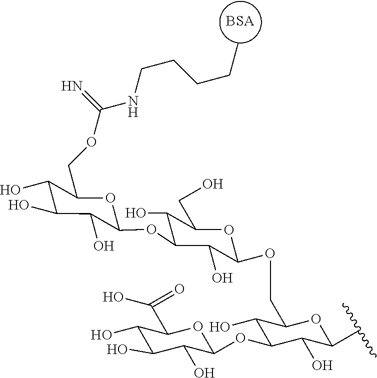 [SMILES: "BSA" ; *C1OC(COC2OC(CO)C(O)C(OC3OC(COC(=N)NCCCCC)C(O)C(O)C3O)C2O)C(O)C(OC2OC(C(=O)O)C(O)C(O)C2O)C1O]

### BSA Imidocarbamate MMW Yeast Glucan Analog BT-1551

To stirring MMW Yeast Glucan (333 mg in 33 mL sterile water) was added CDAP (2.22 mL of 100 mg/mL CDAP in acetonitrile). After 1 minute of stirring, 0.3M triethylamine (3 mL) was added to get pH above 9. The resulting solution was added to a stirring solution of BSA (11 mL, 15 mg/mL BSA in 0.15 M aqueous NaCl) and the resulting solution was stirred 10 min at rt. Ethanolamine (3 mL of 1M) was added and the solution was held overnight at 4° C. This procedure was repeated 5 times and all reactions were pooled into one solution. The solution was dialyzed into sterile PBS using a 500K tangential flow filtration device to afford a solution containing 2.8 g of BT-1551. A dry weight measurement and anthrone provided a glucan:protein ratio of 2:1.

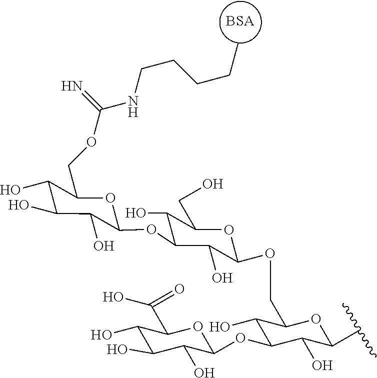 [SMILES: "BSA" ; *C1OC(COC2OC(CO)C(O)C(OC3OC(COC(=N)NCCCCC)C(O)C(O)C3O)C2O)C(O)C(OC2OC(C(=O)O)C(O)C(O)C2O)C1O]

### BSA Imidocarbamate WGP Yeast Glucan Analog BT-1553

To stirring WGP Yeast Glucan (500 mg in 50 mL sterile water) was added CDAP (3.33 mL of 100 mg/mL CDAP in acetonitrile). After 1 minute of stirring, 0.3M triethylamine (5 mL) was added to get pH above 9. The resulting solution was added to a stirring solution of BSA (16.6 mL of a 15 mg/mL BSA in 0.15 M, aqueous NaCl) and the resulting solution was stirred 10 min at rt. Ethanolamine (5 mL of 1M) was added and the solution was held overnight at 4° C. This procedure was repeated 3 times and all reactions were pooled into one solution. The product was washed with sterile water using centrifugation to afford a solution containing BT-1553. Elemental analysis by combustion gave a % N of 6.35%, which translates to a glucan:protein ratio of 1.5:1.

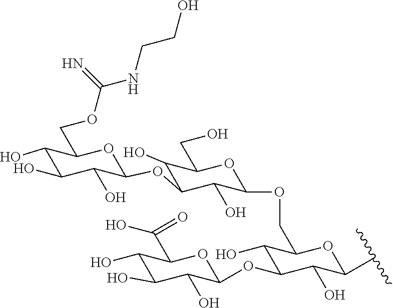 [SMILES: *C1OC(COC2OC(CO)C(O)C(OC3OC(COC(=N)NCCO)C(O)C(O)C3O)C2O)C(O)C(OC2OC(C(=O)O)C(O)C(O)C2O)C1O]

### Ethanolamine Imidocarbamate MMW Yeast Glucan Analog BT-1557

To a stirring solution of MMW Yeast Glucan (50 mg) in sterile water (5 mL) was added CDAP (33 uL of 100 mg/mL CDAP in acetonitrile). After 1 minute of stirring, 0.3 M triethylamine in water (1 mL) was added to get the pH above 9. Ethanolamine (1 mL, 1M aqueous) was and the resulting solution was held overnight at 4° C. The solution was dialyzed into sterile water using a 10K tangential flow filtration device and sterile 0.2 μm filtered to afford a solution containing 28 mg of BT-1557.

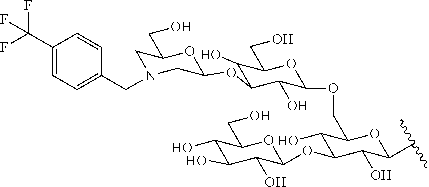 [SMILES: *C1OC(COC2OC(CO)C(O)C(OC3CN(Cc4ccc(C(F)(F)F)cc4)CC(CO)O3)C2O)C(O)C(OC2OC(CO)C(O)C(O)C2O)C1O]

### 4-CF₃—Benzylamine MMW Yeast Glucan Analog BT-1564

The procedure used to synthesize BT-1222 was repeated using 4-trifluoromethylbenzylamine (50 mg) and [O] MMW yeast glucan 1-H (50 mg), with the exceptions that both heating steps were performed at 50° C. and sodium borohydride was used instead of sodium cyanoborohydride, to afford 12 mg of BT-1564. Elemental analysis by combustion gave a % N of 0.46%, which translates to 5.6 mol % 4-trifluoromethylbenzylamine incorporation.

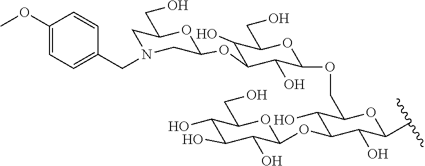 [SMILES: *C1OC(COC2OC(CO)C(O)C(OC3CN(Cc4ccc(OC)cc4)CC(CO)O3)C2O)C(O)C(OC2OC(CO)C(O)C(O)C2O)C1O]

### 4-MeO-Benzylamine MMW Yeast Glucan Analog BT-1565

The procedure used to synthesize BT-1222 was repeated using 4-methoxybenzylamine (50 mg) and [O] MMW yeast glucan 1-H (50 mg), with the exceptions that both heating steps were performed at 50° C. and sodium borohydride was used instead of sodium cyanoborohydride, to afford 8 mg of BT-1565. Elemental analysis by combustion gave a % N of 0.47%, which translates to 5.6 mol % 4-trifluoromethylbenzylamine incorporation.

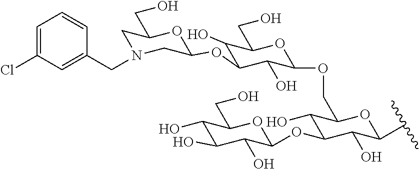 [SMILES: *C1OC(COC2OC(CO)C(O)C(OC3CN(Cc4cccc(Cl)c4)CC(CO)O3)C2O)C(O)C(OC2OC(CO)C(O)C(O)C2O)C1O]

### 3-Cl-Benzylamine MMW Yeast Glucan Analog BT-1572

The procedure used to synthesize BT-1222 was repeated using 3-chlorobenzylamine (50 mg) and [O] MMW yeast glucan 1-L (50 mg), with the exceptions that both heating steps were performed at 50° C. and sodium borohydride was used instead of sodium cyanoborohydride, to afford 14 mg of BT-1572. Elemental analysis by combustion gave a % N of 0.39%, which translates to 4.6 mol % 3-chlorobenzylamine incorporation.

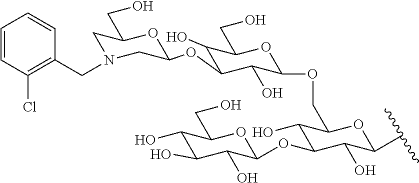 [SMILES: *C1OC(COC2OC(CO)C(O)C(OC3CN(Cc4ccccc4Cl)CC(CO)O3)C2O)C(O)C(OC2OC(CO)C(O)C(O)C2O)C1O]

### 2-Cl-Benzylamine MMW Yeast Glucan Analog BT-1573

The procedure used to synthesize BT-1222 was repeated using 2-chlorobenzylamine (50 mg) and [O] MMW yeast glucan 1-L (50 mg), with the exceptions that both heating steps were performed at 50° C. and sodium borohydride was used instead of sodium cyanoborohydride, to afford 26 mg of BT-1573. Elemental analysis by combustion gave a % N of 0.38%, which translates to 4.5 mol % 2-chlorobenzylamine incorporation.

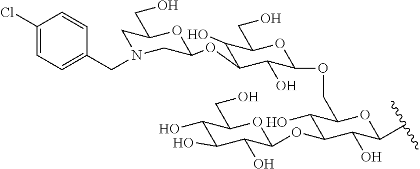 [SMILES: *C1OC(COC2OC(CO)C(O)C(OC3CN(Cc4ccc(Cl)cc4)CC(CO)O3)C2O)C(O)C(OC2OC(CO)C(O)C(O)C2O)C1O]

### 4-Cl-Benzylamine MMW Yeast Glucan Analog BT-1574

The procedure used to synthesize BT-1222 was repeated using 4-chlorobenzylamine (50 mg) and [O] MMW yeast glucan 1-L (50 mg), with the exceptions that both heating steps were performed at 50° C. and sodium borohydride was used instead of sodium cyanoborohydride, to afford 9 mg of BT-1574. Elemental analysis by combustion gave a % N of 0.40%, which translates to 4.8 mol % 4-chlorobenzylamine incorporation.

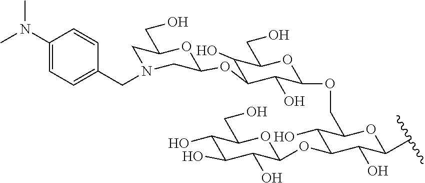 [SMILES: *C1OC(COC2OC(CO)C(O)C(OC3CN(Cc4ccc(N(C)C)cc4)CC(CO)O3)C2O)C(O)C(OC2OC(CO)C(O)C(O)C2O)C1O]

### 4-Me₂N-Benzylamine MMW Yeast Glucan Analog BT-1583

The procedure used to synthesize BT-1222 was repeated using 4-dimethylaminebenzylamine (50 mg) and [O] MMW yeast glucan 1-L (50 mg), with the exceptions that both heating steps were performed at 50° C. and sodium borohydride was used instead of sodium cyanoborohydride, to afford 36 mg of BT-1583. Elemental analysis by combustion gave a % N of 0.22%, which translates to 2.6 mol % 4-dimethylaminebenzylamine incorporation.

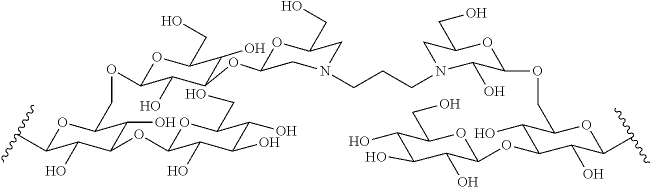 [SMILES: *C1OC(COC2OC(CO)C(O)C(OC3CN(CCCN4CC(CO)OC(OCC5OC(*)C(O)C(OC6OC(CO)C(O)C(O)C6O)C5O)C4O)CC(CO)O3)C2O)C(O)C(OC2OC(CO)C(O)C(O)C2O)C1O]

### Diaminopropane Crosslinked MMW Yeast Glucan Analog BT-1584

The procedure used to synthesize BT-1222 was repeated using the amine in diaminopropane MMW yeast glucan analog BT-1253 (50 mg) instead of benzyl amine and [O] MMW yeast glucan 1-L (50 mg), with the exceptions that both heating steps were performed at 50° C., to afford 30 mg of BT-1584. Elemental analysis by combustion gave a % N of 0.24%, which translates to 1.4 mol % incorporation of the bis-morpholine propane adduct.

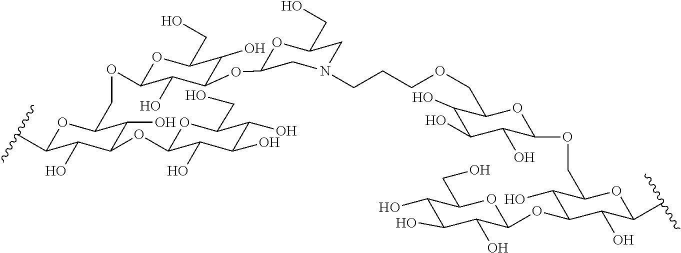 [SMILES: *C1OC(COC2OC(COCCCN3CC(CO)OC(OC4C(O)C(CO)OC(OCC5OC(*)C(O)C(OC6OC(CO)C(O)C(O)C6O)C5O)C4O)C3)C(O)C(O)C2O)C(O)C(OC2OC(CO)C(O)C(O)C2O)C1O]

### Propylamino Crosslinked MMW Yeast Glucan Analog BT-1587

The procedure used to synthesize BT-1222 was repeated using the amine in propyl amine MMW yeast glucan analog BT-1267 (50 mg) instead of benzyl amine and [O] MMW yeast glucan 1-L (50 mg), with the exceptions that both heating steps were performed at 50° C. and sodium borohydride was used instead of sodium cyanoborohydride, to afford 28 mg of BT-1587. Elemental analysis by combustion gave a % N of 0.29%, which translates to 3.5 mol % incorporation of the propyl amine morpholine adduct.

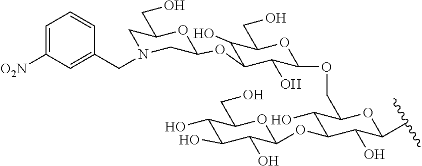 [SMILES: *C1OC(COC2OC(CO)C(O)C(OC3CN(Cc4cccc([N+](=O)[O-])c4)CC(CO)O3)C2O)C(O)C(OC2OC(CO)C(O)C(O)C2O)C1O]

### 3-NO₂—Benzylamine MMW Yeast Glucan Analog BT-1592

The procedure used to synthesize BT-1222 was repeated using 3-nitrobenzylamine (50 mg) and [O] MMW yeast glucan 1-L (50 mg), with the exception that both heating steps were performed at 50° C., to afford 41 mg of BT-1592. Elemental analysis by combustion gave a % N of 0.41%, which translates to 2.4 mol % 3-nitrobenzylamine incorporation.

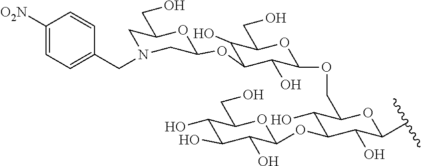 [SMILES: *C1OC(COC2OC(CO)C(O)C(OC3CN(Cc4ccc([N+](=O)[O-])cc4)CC(CO)O3)C2O)C(O)C(OC2OC(CO)C(O)C(O)C2O)C1O]

### 4-NO₂—Benzylamine MMW Yeast Glucan Analog BT-1593

The procedure used to synthesize BT-1222 was repeated using 4-nitrobenzylamine (50 mg) and [O] MMW yeast glucan 1-L (50 mg), with the exception that both heating steps were performed at 50° C., to afford 36 mg of BT-1593. Elemental analysis by combustion gave a % N of 0.44%, which translates to 2.6 mol % 4-nitrobenzylamine incorporation.

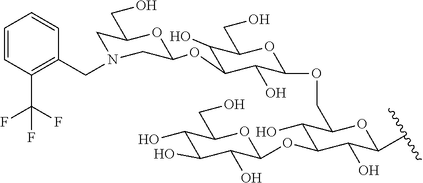 [SMILES: *C1OC(COC2OC(CO)C(O)C(OC3CN(Cc4ccccc4C(F)(F)F)CC(CO)O3)C2O)C(O)C(OC2OC(CO)C(O)C(O)C2O)C1O]

### 2-CF₃—Benzylamine MMW Yeast Glucan Analog BT-1594

The procedure used to synthesize BT-1222 was repeated using 2-trifluoromethylbenzylamine (50 mg) and [O] MMW yeast glucan 1-L (50 mg), with the exceptions that both heating steps were performed at 50° C. and sodium borohydride was used instead of sodium cyanoborohydride, to afford 35 mg of BT-1594. Elemental analysis by combustion gave a % N of 0.28%, which translates to 3.3 mol % 2-trifluoromethylbenzylamine incorporation.

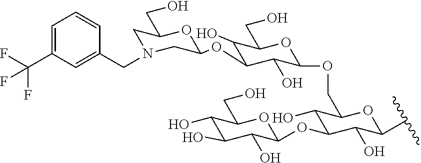 [SMILES: *C1OC(COC2OC(CO)C(O)C(OC3CN(Cc4cccc(C(F)(F)F)c4)CC(CO)O3)C2O)C(O)C(OC2OC(CO)C(O)C(O)C2O)C1O]

### 3-CF₃—Benzylamine MMW Yeast Glucan Analog BT-1595

The procedure used to synthesize BT-1222 was repeated using 3-trifluoromethylbenzylamine (50 mg) and [O] MMW yeast glucan 1-L (50 mg), with the exceptions that both heating steps were performed at 50° C. and sodium borohydride was used instead of sodium cyanoborohydride, to afford 35 mg of BT-1595. Elemental analysis by combustion gave a % N of 0.25%, which translates to 3.0 mol % 3-trifluoromethylbenzylamine incorporation.

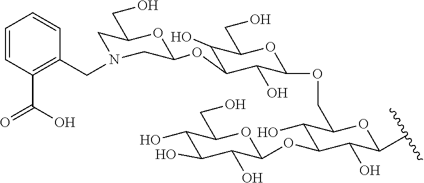 [SMILES: *C1OC(COC2OC(CO)C(O)C(OC3CN(Cc4ccccc4C(=O)O)CC(CO)O3)C2O)C(O)C(OC2OC(CO)C(O)C(O)C2O)C1O]

### 2-Aminomethylbenzoic Acid MMW Yeast Glucan Analog BT-1597

The procedure used to synthesize BT-1222 was repeated using 2-aminomethylbenzoic acid (50 mg) and [O] MMW yeast glucan 1-L (50 mg), with the exception that both heating steps were performed at 50° C., to afford 37 mg of BT-1597. Elemental analysis by combustion gave a % N of 0.10%, which translates to 1.2 mol % 2-aminomethylbenzoic acid incorporation.

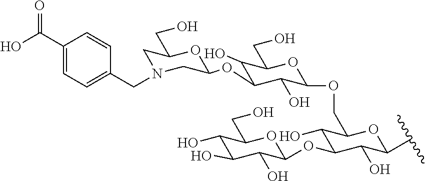 [SMILES: *C1OC(COC2OC(CO)C(O)C(OC3CN(Cc4ccc(C(=O)O)cc4)CC(CO)O3)C2O)C(O)C(OC2OC(CO)C(O)C(O)C2O)C1O]

### 4-Aminomethylbenzoic Acid MMW Yeast Glucan Analog BT-1598

The procedure used to synthesize BT-1222 was repeated using 4-aminomethylbenzoic acid (50 mg) and [O] MMW yeast glucan 1-L (50 mg), with the exception that both heating steps were performed at 50° C., to afford 36 mg of BT-1598. Elemental analysis by combustion gave a % N of 0.24%, which translates to 2.9 mol % 4-aminomethylbenzoic acid incorporation.

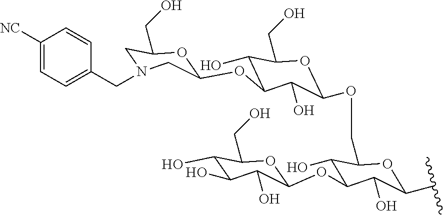 [SMILES: *C1OC(COC2OC(CO)C(O)C(OC3CN(Cc4ccc(C#N)cc4)CC(CO)O3)C2O)C(O)C(OC2OC(CO)C(O)C(O)C2O)C1O]

### 4-Aminomethylbenzonitrile MMW Yeast Glucan Analog BT-1599

The procedure used to synthesize BT-1222 was repeated using 4-aminomethylbenzonitrile (50 mg) and [O] MMW yeast glucan 1-L (50 mg), with the exception that both heating steps were performed at 50° C., to afford 33 mg of BT-1599. Elemental analysis by combustion gave a % N of 0.38%, which translates to 2.2 mol % 4-aminomethylbenzonitrile incorporation.

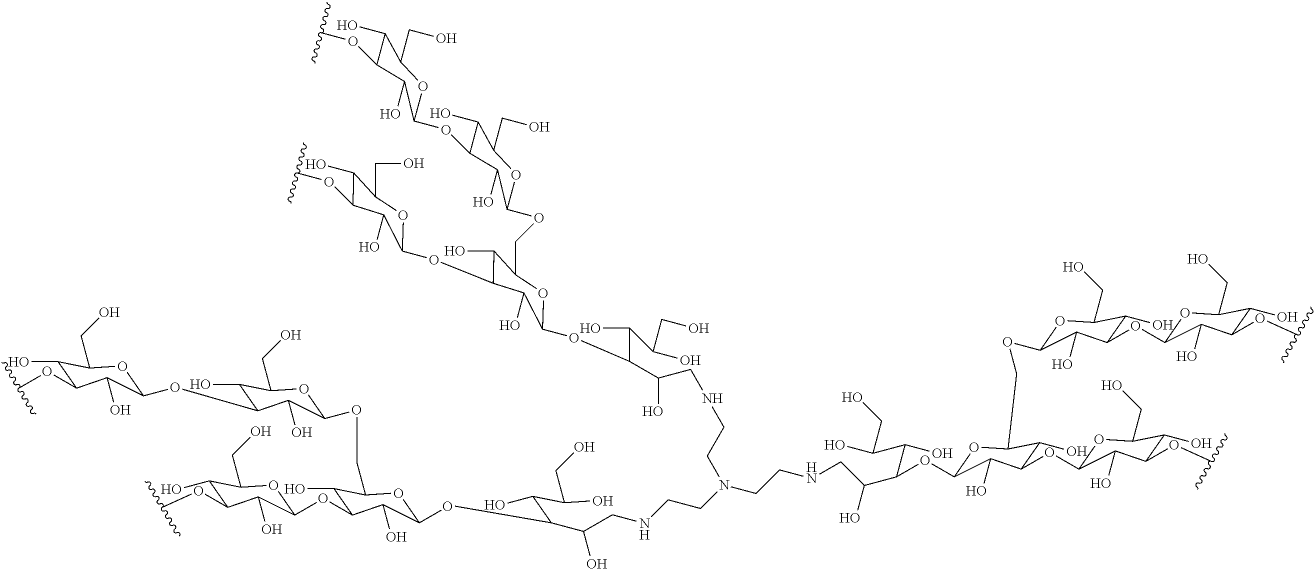 [SMILES: *OC1C(O)C(CO)OC(OC2C(O)C(CO)OC(OCC3OC(OC(C(O)CNCCN(CCNCC(O)C(OC4OC(COC5OC(CO)C(O)C(OC6OC(CO)C(O)C(O*)C6O)C5O)C(O)C(OC5OC(CO)C(O)C(O*)C5O)C4O)C(O)C(O)CO)CCNCC(O)C(OC4OC(COC5OC(CO)C(O)C(OC6OC(CO)C(O)C(O*)C6O)C5O)C(O)C(OC5OC(CO)C(O)C(O*)C5O)C4O)C(O)C(O)CO)C(O)C(O)CO)C(O)C(OC4OC(CO)C(O)C(O*)C4O)C3O)C2O)C1O]

### Triamine MMW Yeast Glucan Analog BT-1600

To MMW yeast glucan (100 mg) and sterile water (2.2 mL) was added tris-2 aminoethyl amine (0.2 mL of 10 mg/mL in water). This solution was stirred at 65° C. for 24 hours. Sodium cyanoborohydride (100 mg) was then added to the solution and it was stirred at 65° C. for 6 days. An OPA assay showed that 54% of the amines had reacted. The solution was dialyzed into sterile water over a 3K centrifugal filtration device to afford 46 mg of BT-1600. Elemental analysis by combustion gave a % N of 0.19%, which translates to 0.6 mol % tris-2 aminoethyl amine incorporation.

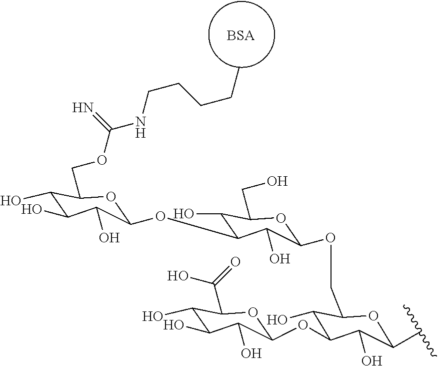 [SMILES: "BSA" ; *C1OC(COC2OC(CO)C(O)C(OC3OC(COC(=N)NCCCCC)C(O)C(O)C3O)C2O)C(O)C(OC2OC(C(=O)O)C(O)C(O)C2O)C1O]

### BSA LMW Yeast Glucan Analog BTH-1601

To stirring LMW Yeast Glucan (50 mg in 6.8 mL sterile water) was added CDAP (0.333 mL of 100 mg/mL CDAP in acetonitrile). After 1 minute of stirring, 0.3M triethylamine (500 uL) was added to get the pH above 9. The resulting solution was added to a stirring solution of aqueous BSA (1.66 mL of 15 mg/mL BSA in 0.15 M saline) and the resulting solution was stirred for 10 min at rt. Ethanolamine (1 mL of 1M) was added and the solution was held overnight at 4° C. The solution was dialyzed into sterile PBS using a 10K tangential flow filtration device and sterile 0.2 μm filtered to afford a solution containing 27 mg of BT-1601. A 2.3:1 ratio of glucan:protein was found by anthrone and 280 nm absorbance.

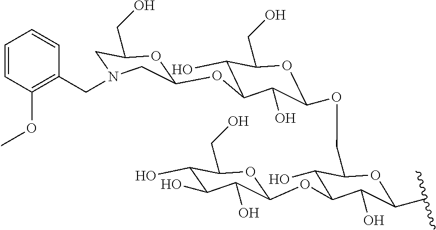 [SMILES: *C1OC(COC2OC(CO)C(O)C(OC3CN(Cc4ccccc4OC)CC(CO)O3)C2O)C(O)C(OC2OC(CO)C(O)C(O)C2O)C1O]

### 2-MeO-Benzylamine MMW Yeast Glucan Analog BT-1622

The procedure used to synthesize BT-1222 was repeated using 2-methoxybenzylamine (50 mg) and [O] MMW yeast glucan 1-L (50 mg), with the exceptions that both heating steps were performed at 50° C. and sodium borohydride was used instead of sodium cyanoborohydride, to afford 33 mg of BT-1622. Elemental analysis by combustion gave a % N of 0.28%, which translates to 3.3 mol % 2-methoxybenzylamine incorporation.

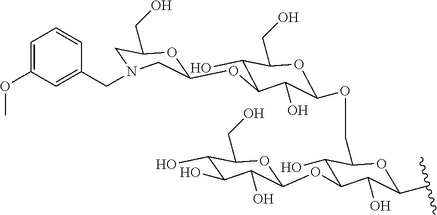 [SMILES: *C1OC(COC2OC(CO)C(O)C(OC3CN(Cc4cccc(OC)c4)CC(CO)O3)C2O)C(O)C(OC2OC(CO)C(O)C(O)C2O)C1O]

### 3-MeO-Benzylamine MMW Yeast Glucan Analog BT-1623

The procedure used to synthesize BT-1222 was repeated using 3-methoxybenzylamine (50 mg) and [O] MMW yeast glucan 1-L (50 mg), with the exceptions that both heating steps were performed at 50° C. and sodium borohydride was used instead of sodium cyanoborohydride, to afford 33 mg of BT-1623. Elemental analysis by combustion gave a % N of 0.22%, which translates to 2.6 mol % 3-methoxybenzylamine incorporation.

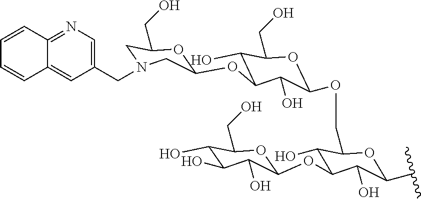 [SMILES: *C1OC(COC2OC(CO)C(O)C(OC3CN(Cc4cnc5ccccc5c4)CC(CO)O3)C2O)C(O)C(OC2OC(CO)C(O)C(O)C2O)C1O]

### 2-Aminoquinoline MMW Yeast Glucan Analog BT-1624

The procedure used to synthesize BT-1222 was repeated using 2-aminoquinoline (50 mg) and [O] MMW yeast glucan 1-L (50 mg), with the exception that both heating steps were performed at 50° C., to afford 33 mg of BT-1624. Elemental analysis by combustion gave a % N of 0.12%, which translates to 0.7 mol % 2-aminoquinoline incorporation.

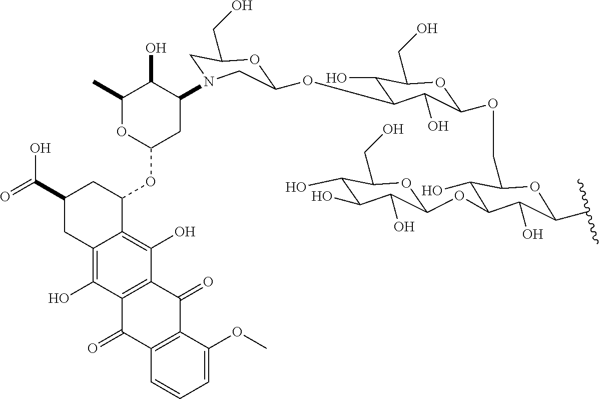

### Doxorubicin MMW Yeast Glucan Analog BT-1631

The procedure used to synthesize BT-1222 was repeated using doxorubicin (5 mg) and [O] MMW yeast glucan 1-L (50 mg), with the exception that both heating steps were performed at 50° C., to afford 19 mg of BT-1631. Elemental analysis by combustion gave a % N of 0.17%, which translates to 2.1 mol % doxorubicin incorporation.

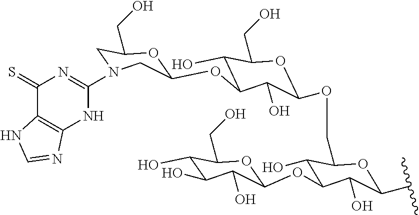 [SMILES: *C1OC(COC2OC(CO)C(O)C(OC3CN(c4nc(=S)c5[nH]cnc5[nH]4)CC(CO)O3)C2O)C(O)C(OC2OC(CO)C(O)C(O)C2O)C1O]

### Thioguanine MMW Yeast Glucan Analog BT-1632

The procedure used to synthesize BT-1222 was repeated using 2-amino-7H-purine-6-thiol (50 mg) and [O] MMW yeast glucan 1-L (50 mg), with the exception that both heating steps were performed at 50° C., to afford 36 mg of BT-1632. Elemental analysis by combustion gave a % N of 0.19%, which translates to 0.4 mol % 2-amino-7H-purine-6-thiol incorporation.

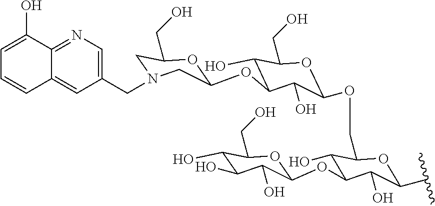 [SMILES: *C1OC(COC2OC(CO)C(O)C(OC3CN(Cc4cnc5c(O)cccc5c4)CC(CO)O3)C2O)C(O)C(OC2OC(CO)C(O)C(O)C2O)C1O]

### 2-Amino-8-Hydroxyquinoline MMW Yeast Glucan Analog BT-1633

The procedure used to synthesize BT-1222 was repeated using 2-Amino-8-hydroxyquinoline (50 mg) and [O] MMW yeast glucan 1-L (50 mg), with the exception that both heating steps were performed at 50° C., to afford 15 mg of BT-1633. Elemental analysis by combustion gave a % N of 1.93%, which translates to 12.1 mol % 2-amino-8-hydroxyquinoline incorporation.

### Oxidized VHMW Yeast Glucan 6-L and 6-H

To an aqueous solution of VHMW yeast glucan of known concentration was added an aqueous 25 mg/mL NaIO₄ solution to obtain a ratio of either 0.08 mg or 0.12 mg of NaIO₄ per mg of glucan. The final reaction concentration, with respect to glucan, was adjusted to 10 mg/mL with sterile water. The reactions were swirled and mixed to dissolve all reagent and the reactions was placed in the dark for 20 hours. The oxidized products, 6-L and 6-H, were carried directly into the reductive amination step without purification and correspond to 0.08 mg and 0.12 mg of NaIO₄ per mg of glucan respectively.

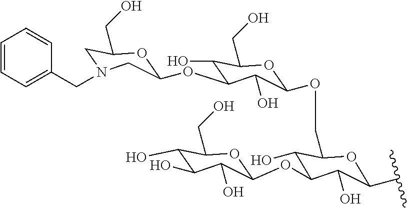 [SMILES: *C1OC(COC2OC(CO)C(O)C(OC3CN(Cc4ccccc4)CC(CO)O3)C2O)C(O)C(OC2OC(CO)C(O)C(O)C2O)C1O]

### Benzyl Amine VHMW Yeast Glucan Analogs BT-1629 and BT-1630

The procedure used to synthesize BT-1222 was repeated using benzyl amine and [O] VHMW yeast glucan 6-L (100 mg) and 6-H (100 mg), with the exception of using 10K tangential flow filtration instead of centrifugal filtration, to afford 53 mg of BT-1629 and 26 mg of BT-1630 respectively. Elemental analysis by combustion gave a % N of 0.19% and 0.24% respectively, which translates to 2.2 mol % benzyl amine for BT-1629 and 2.8 mol % benzyl amine for BT-1630.

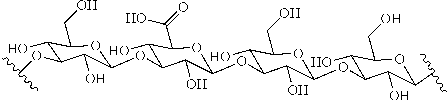 [SMILES: *OC1C(O)C(CO)OC(OC2C(O)C(OC3C(O)C(CO)OC(OC4C(O)C(*)OC(CO)C4O)C3O)OC(C(=O)O)C2O)C1O]

### TEMPO Oxidized Curdlan BT-1354

To a suspension of Curdlan (2 g) in sterile water (200 mL) was added (2,2,6,6-Tetramethylpiperidin-1-yl)oxyl (36 mg) and sodium bromide (200 mg). While the solution was stirring, aqueous sodium hydroxide (2M) was added to raise the pH above 11. Then aqueous sodium hypochlorite (7.1 mL, 12.5%) was added to the mixing solution. Additional NaOH was added during the reaction to keep the pH above 10. Once the solution had a stable pH of 11, sodium borohydride (400 mg) was added and stirred 30 min at rt. The resulting homogeneous solution was precipitated with an equal volume of acetone and the precipitate was collected via centrifugation and dried in a vacuum oven to afford a white solid. The solid was dissolved in sterile water by heating to 50° C. and mixing. The solution was neutralized to a pH of 7 with HCl, 1.2 micron filtered, and 0.45 micron filtered to afford a solution containing 1.6 g of BT-1354. The presence of acid was confirmed by the same method used for BT-1407.

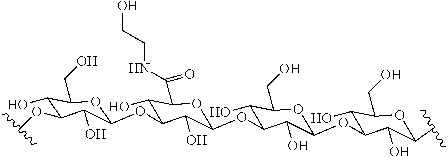 [SMILES: *OC1C(O)C(CO)OC(OC2C(O)C(OC3C(O)C(CO)OC(OC4C(O)C(*)OC(CO)C4O)C3O)OC(C(=O)NCCO)C2O)C1O]

### Hydroxyethyl Amide Curdlan Analog 163-006B

The procedure used to synthesize BT-1512 was repeated using 1-amino-2-ethanol (0.091 mL) and TEMPO oxidized Curdlan Analog BT-1354 (25 mg) to afford 20 mg of 163-006B that contained 3.7 weight % glucuronic acid. The starting acid, BT-1512, contained 6.9 weight % acid, indicating a 46% conversion to the amide.

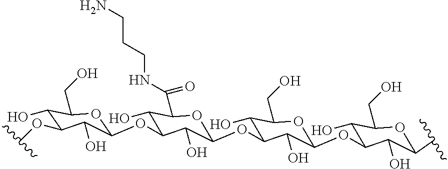 [SMILES: *OC1C(O)C(CO)OC(OC2C(O)C(OC3C(O)C(CO)OC(OC4C(O)C(*)OC(CO)C4O)C3O)OC(C(=O)NCCCN)C2O)C1O]

### Aminopropyl Amide Curdlan Analog 163-028

The procedure used to synthesize BT-1512 was repeated using 1,3-diaminopropane (0.039 mL) and TEMPO oxidized Curdlan Analog BT-1354 (40 mg) to afford 24 mg of 163-028. An OPA assay showed that the analog contained 14.9 mole % primary amine.

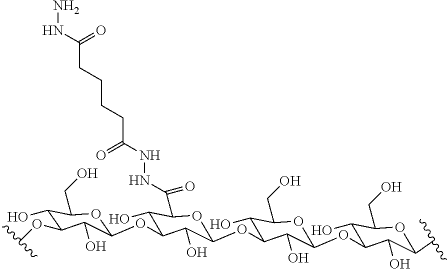 [SMILES: *OC1C(O)C(CO)OC(OC2C(O)C(OC3C(O)C(CO)OC(OC4C(O)C(*)OC(CO)C4O)C3O)OC(C(=O)NNC(=O)CCCCC(=O)NN)C2O)C1O]

### Aminopropyl Amide Curdlan Analog 163-071C

The procedure used to synthesize BT-1512 was repeated using adipic dihydrazide (2.0 g) and TEMPO oxidized Curdlan Analog BT-1354 (100 mg) to afford 73 mg of 163-071C in which 43% of the acid residues had reacted, as measured by the loss of acid residues in a phenyl hydrazine fluorescence assay.

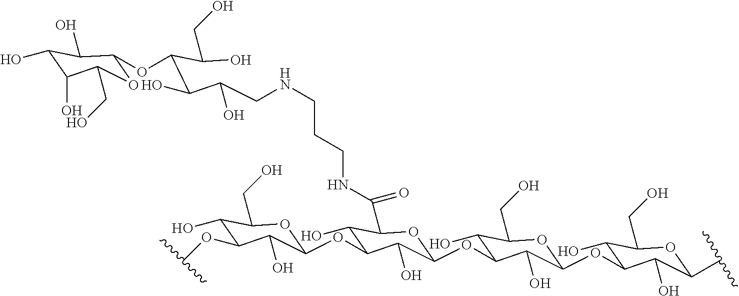 [SMILES: *OC1C(O)C(CO)OC(OC2C(O)C(OC3C(O)C(CO)OC(OC4C(O)C(*)OC(CO)C4O)C3O)OC(C(=O)NCCCNCC(O)C(O)C(OC3OC(CO)C(O)C(O)C3O)C(O)CO)C2O)C1O]

### Lactose Aminopropyl Amide Curdlan Analog 163-057A

To Aminopropyl Amide Curdlan Analog 163-028 (6 mg) in sterile water (14 mL) was added lactose (210 mg), and sodium cyanoborohydride (125 mg). The resulting mixture was placed in a 50° C. water bath for 1 day. The reaction was cooled and additional sodium cyanoborohydride (125 mg) was added and the reaction was held at 50° C. for 2 days. The reaction was cooled to room temperature and an equivalent volume of acetone was added to precipitate the product. The resulting pellet was washed with acetone, dried in a vacuum oven, and dissolved in sterile water to afford a solution containing 4 mg of 163-057A. An OPA assay showed the level of primary amine had decreased from 14.0 mole % to 0.4 mole %, indicating a 97% conversion.

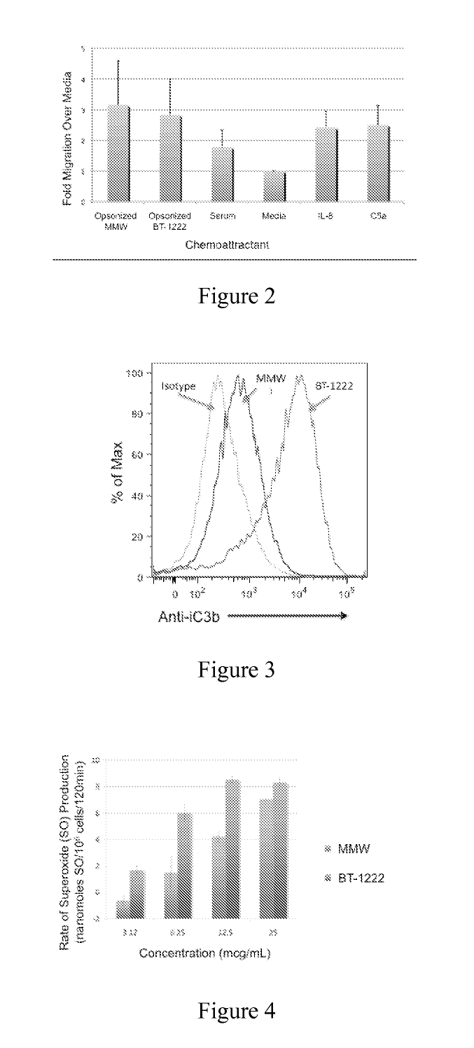

FIG. 2 demonstrates that derivatization of β-glucans do not negatively affect the immune cells to migrate. This ability was measured by comparing the migratory capacity of human neutrophils to parent medium molecular weight (MMW) yeast β-glucan versus one of its derivatives, BT-1222. Neutrophils migrate in a comparable manner to both the parent MMW and BT-1222. In contrast, the migration of neutrophils to MMW β-glucan or BT-1222 was 3.1 fold and 2.8 fold over medium alone, respectively.

### Migration Methodology:

Heparinized peripheral blood was obtained from healthy volunteers, and neutrophils were isolated by density gradient centrifugation with Ficoll-Paque followed by 3% dextrose sedimentation. Residual erythrocytes were removed by hypotonic lysis. Neutrophils were resuspended at 200,000 cells/ml in RPMI with 3% autologous serum (AS). MMW (3.3 mg/mL), BT-1222 (3.3 mg/mL) and PBS control were mixed with AS in 1:1 ratio and placed in a 37° C. water bath for 30 minutes. After incubation, Imprime and benzylamine-Imprime were diluted in RPMI to achieve 100, 50, 25, and 10 μg/ml concentrations with final concentration of serum being 3%. PBS control was diluted in a similar manner. 31 μl of each dilution of MMW, BT-1222 or PBS control were added to 3 wells in the bottom well of Neuroprobe chemotaxis plates. Chemoattractants, C5a and IL-8 at 50 ng/mL served as positive controls. The 8 μm chemotaxis membrane was applied to the plate and 6×10³ number of neutrophils in 30 μl volume were added over each well containing chemoattractants. The plate was placed in 37° C. incubator for 1 hour, lysed with 10 μl Cell-titer glo and read for luminescence on the M5 plate reader. Fold over media migration (chemotaxis) was calculated by dividing the RLU (relative luminescence units) for the treatment groups over migration to media (chemokinesis).

FIG. 3 demonstrates that the ability of certain derivatives of β-glucans to activate complement is higher than that of the parent glucan. This ability was evaluated by comparing the levels of iC3b complement fragment detected on human neutrophils with the derivative versus parent β-glucan-bound. The histogram shows that staining of iC3b on BT-1222-bound neutrophils is higher than that on MMW-bound neutrophils.

iC3b Deposition Analysis Methodology:

Enriched neutrophils were resuspended at 1×10⁶ cells/mL in RPMI 1640 supplemented with 10% serum. The parent or the derivatized glucans at 200, μg/mL hexose concentration were added to neutrophils and incubated in a 37° C., 5% C02 humidified incubator for 2 hr. After incubation, cells were washed twice with FACS buffer (HBSS supplemented with 1% FBS and 0.1% sodium azide) to remove any unbound β-glucan, stained with with a neo-epitope specific anti-iC3b Ab and PE-conjugated goat anti-mouse IgG and and subsequently analyzed by flow cytometry.

Table 1 below lists the fold change of mean fluorescence intensity (MFI) values of iC3b staining on neutrophils treated with each of the parent or derivatized glucans over that of PBS control-treated cells.

TABLE 1
iC3b deposition on neutrophils (Compound vs. Parent MFIs)

| | MFI_{(ic3b)} | Approximate Fold |
|---|---|---|
| | compound/MFI_{(ic3b)} | Increase/Decrease in MFI |
| Compounds | PBS | over Parent Compound |
| Medium molecular | 2.0 ± 0.7 | |
| weight yeast β-glucan | | |
| (MMW, parent) | | |
| BT-1222 | 5.9 ± 2.2 | 3.0 |
| BT-1275 | 1.7 ± 0.1 | 0.9 |
| BT-1220 | 10.1 | 5.1 |
| BT-1297 | 2.3 | 1.2 |
| BT-1304 | 1.2 | 0.6 |
| BT-1300 | 1.6 | 0.8 |
| BT-1267 | 8.0 ± 3.9 | 4.0 |
| BT-1248 | 3.5 ± 0.4 | 1.8 |
| BT-1244 | 3.0 ± 0.1 | 1.5 |
| BT-1243 | 8.6 ± 0.3 | 4.3 |
| BT-1245 | 4.5 ± 1.2 | 2.3 |
| Low molecular weight | 1.1 | |
| yeast β-glucan | | |
| (LMW, parent) | | |
| BT-1276 | 1.2 ± 0.1 | 1.1 |
| BT-1273 | 2.4 | 2.2 |
| BT-1277 | 1.0 | 0.9 |
| BT-1278 | 1.0 | 0.9 |
| Laminarin (parent) | 1.3 | |
| BT-1234 | 5.1 | 3.9 |
| BT-1281 | 1.1 | 0.8 |
| Scleroglucan (parent) | 1.6 | |
| BT-1272 | 3.9 | 2.4 |
| BT-1290 | 1.1 | 0.7 |
| Dextran (control) | 1.1 ± 0.2 | |
| BT-1266 | 1.0 | 0.9 |
| BT-1287 | 1.0 | 0.9 |
| BT-1286 | 1.0 | 0.9 |

FIG. 4 below demonstrates higher binding of certain derivatives of β-glucans as compared to the parent b-glucan on human neutrophils. This ability was evaluated by comparing the levels of neutrophil-bound parent versus the derivatives of β-glucans detected by BfD IV, a monoclonal antibody (MAb) specific for β-1,3/1,6 glucans. FIG. 4 shows flow cytometric detection of BfD IV staining on neutrophils that have been allowed to bind either the MMW or BT-1222. The histogram shows that staining of BfD IV on BT-1222-bound neutrophils is higher than that on MMW-bound neutrophils. Table 2 lists the fold change of MFI values of BfD IV staining on neutrophils treated with each of the parent and derivatized glucans over that of PBS control-treated cells.

β-Glucan Binding Analysis Methodology:

Enriched neutrophils were resuspended at 1×10⁶ cells/mL in RPMI 1640 supplemented with 10% serum. The parent or the derivatized glucans at 200, μg/mL hexose concentration were added to neutrophils and incubated in a 37° C., 5% CO₂ humidified incubator for 2 hr. After incubation, cells were washed twice with FACS buffer (HBSS supplemented with 1% FBS and 0.1% sodium azide) to remove any unbound β-glucan, and subsequently treated with Fc block. Post Fc block step, cells were stained with the BfD IV Ab for 30 min at 4° C. and washed twice with cold FACS buffer. Cells were then incubated with FITC-conjugated F(ab′)2 goat anti-mouse IgM for 30 min at 4° C. and washed once with cold FACS buffer before fixing with 1% paraformaldehyde. Events were collected on a LSRII flow cytometer and analysis was performed using FlowJo software.

Table 2 summarizes the fold change of MFI values of BfD IV staining on neutrophils treated with each of the parent, or derivatized β-glucans, over that of PBS control-treated cells.

TABLE 2
β-glucan binding on neutrophils (Compound vs. Parent MFIs)

| | MFI_{(BID IV)} | |
|---|---|---|
| | compound/ | Approximate Fold |
| | MFI_{(BID IV)} | Increase/Decrease in MFI |
| Compounds | PBS | over Parent Compound |
| Medium molecular | 4.2 ± 2.2 | |
| weight yeast β-glucan | | |
| (MMW, parent) | | |
| BT-1222 | 13.2 ± 3.6 | 3.1 |
| BT-1275 | 2.4 ± 1.2 | 0.6 |
| BT-1220 | 20.3 | 4.8 |
| BT-1297 | 2.2 | 0.5 |
| BT-1304 | 2.1 | 0.5 |
| BT-1300 | 2.5 | 0.6 |
| BT-1267 | 23.6 ± 16.4 | 5.6 |
| BT-1248 | 4.7 ± 3.8 | 1.1 |
| BT-1244 | 4.0 ± 1.9 | 1.0 |
| BT-1243 | 19.7 ± 13.4 | 4.7 |
| BT-1245 | 6.6 ± 3.78 | 1.6 |
| Low molecular weight | 1.5 ± 0.4 | |
| yeast β-glucan (LMW, parent) | | |
| BT-1276 | 1.5 ± 0.1 | 1.0 |
| BT-1273 | 17.0 | 11.3 |
| BT-1277 | 1.3 | 0.9 |
| BT-1278 | 1.3 | 0.9 |
| Laminarin (parent) | 1.1 | |
| BT-1234 | 77 | 70.0 |
| BT-1281 | 1.2 | 1.1 |
| Scleroglucan (parent) | 1.9 | |
| BT-1272 | 3.2 | 1.7 |
| BT-1290 | 1.4 | 0.7 |
| Dextran (control) | 1.0 | |
| BT-1266 | 1.1 | 1.1 |
| BT-1287 | 1.0 | 1.0 |
| BT-1286 | 1.0 | 1.0 |

FIG. 4 demonstrates that both modified and derivatives of β-glucans can induce a higher oxidative burst response in immune cells as compared to the parent β-glucan. Both parent MMW and derivativatized BT-1222 induced a dose dependent oxidative burst as measured by rate of superoxide production, but in comparison to MMW, the rate of superoxide production was higher for the derivative at each concentration tested.

Oxidative Burst Methodology:

Dilutions of MMW and BT-1222 were prepared out-of-plate in water. Subsequently, 50 μL of each dilution were added to triplicate wells of Costar® Universal-Bind™ microtiter plates. For immobilizing the glucans on the plate, the plate was irradiated for 5 min in a UV cross-linker, and then incubated at 50° C. until completely dried. After checking for complete dryness of the plate, it was cross-linked again under UV for 5 minutes. The microtiter plates were then blocked with 0.25% bovine serum albumin (BSA) in DPBS for 30 min at room temperature. Each microtiter plate included positive control wells and negative control wells to which only water with no polysaccharide was added at this point.

The oxidative burst response was then determined by standard methods by measuring SO production through the reduction of cytochrome c. Peripheral blood mononuclear cells (PBMCs) were resuspended in HBSS/HEPES buffer with 0.25% BSA at a concentration of 4×10⁶ cells/mL and maintained at 37° C. until added to the plate. A 100 μL aliquot of 200 μM bovine cytochrome c solution in HBSS/HEPES buffer previously incubated at 37° C. was added to each well. Approximately 4.0×10⁵ cells, in a 100 μL volume, were subsequently added to each well of the microtiter plate. The negative assay control consisting of cells and cytochrome c was added in the uncoated negative control wells. As a positive assay control, just before reading the plate, phorbol myristate acetate (PMA) at 100 ng/mL was added as the stimulus to the cells in the uncoated positive control wells to achieve a final concentration of 50 ng/mL. The plate was maintained at 37° C., and optical density (OD) in each well was read at 550 nm every 15 min for 120 min using a spectrophotometer. For each concentration of polysaccharide, the OD change (ΔOD) was calculated from the time of minimum response (1 min) to the time of maximum response (120 mins). The rate of SO production (nmoles of SO/106 cells/120 mins) was calculated from the ΔOD value and the extinction coefficient of cytochrome c (21.1×103 M-1 cm-1).

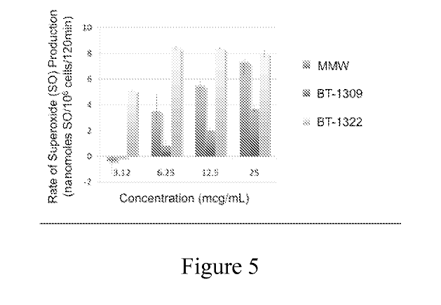

FIG. 5 demonstrates that the parent scleroglucan (BT-1309) when modified by debranching (BT-1322), induces higher oxidative burst than the parent scleroglucan and also the parent MMW yeast β-glucan.

Oxidative Burst Methodology:

Dilutions of MMW, parent scleroglucan (BT-1309), and debranched scleroglucan (BT-1322) were prepared out-of-plate in water. Subsequently, 50 μL of each dilution were added to triplicate wells of Costar® Universal-Bind™ microtiter plates. For immobilizing the glucans on the plate, the plate was irradiated for 5 min in a UV cross-linker, and then incubated at 50° C. until completely dried. After checking for complete dryness of the plate, it was cross-linked again under UV for 5 minutes. The microtiter plates were then blocked with 0.25% bovine serum albumin (BSA) in DPBS for 30 min at room temperature. Each microtiter plate included positive control wells and negative control wells to which only water with no polysaccharide was added at this point.

The oxidative burst response was then determined by standard methods by measuring SO production through the reduction of cytochrome c. Peripheral blood mononuclear cells (PBMCs) were resuspended in HBSS/HEPES buffer with 0.25% BSA at a concentration of 4×10⁶ cells/mL and maintained at 37° C. until added to the plate. A 100 μL aliquot of 200 μM bovine cytochrome c solution in HBSS/HEPES buffer previously incubated at 37° C. was added to each well. Approximately 4.0×10⁵ cells, in a 100 μL volume, were subsequently added to each well of the microtiter plate. The negative assay control consisting of cells and cytochrome c was added in the uncoated negative control wells. As a positive assay control, just before reading the plate, phorbol myristate acetate (PMA) at 100 ng/mL was added as the stimulus to the cells in the uncoated positive control wells to achieve a final concentration of 50 ng/mL. The plate was maintained at 37° C., and optical density (OD) in each well was read at 550 nm every 15 min for 120 min using a spectrophotometer. For each concentration of polysaccharide, the OD change (ΔOD) was calculated from the time of minimum response (1 min) to the time of maximum response (120 mins). The rate of SO production (nmoles of SO/106 cells/120 mins) was calculated from the ΔOD value and the extinction coefficient of cytochrome c (21.1×103 M-1 cm-1).

The modification and derivatization of β-glucans may also be combined such that any b-glucan can be modified and derivatized to increase its activity.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-41. (canceled)

42. A composition comprising a chemically modified beta 1,3 linked glucose polymer of at least 3 glucose residues having a backbone with at least one glucose residue linked to the backbone via a beta 1,6 linkage resulting in a sidechain, wherein at least one glucose residue containing a vicinal diol within the polymer has been transformed into a substituted morpholine or [1,4]-oxazepane via periodate oxidation and reductive amination with a protein.

43. The composition of claim 42 wherein protein is bovine serum albumin.

44. The composition f claim 42 wherein the protein comprises an antibody.

45. The composition of claim 44 herein the antibody is used as a tumor therapy.

46. The composition of claim 45 wherein the antibody is cetuximab.

47. A composition comprising a chemically modified beta 1,3 linked glucose polymer of at least 3 glucose residues having at least one hydroxyl group of the polymer linked to a second amine via an imidocarbamate linkage formed via cyanogen bromide or 1-cyano-4-dimethylaminopyridinium tetrafluoroborate.

48. The composition of claim 47 wherein the second amine is a peptide or protein.

49. The composition of claim 48 wherein the protein is bovine serum albumin.

50. The composition of claim 47 wherein the protein is an antibody.

51. A composition comprising a chemically modified beta 1,3 linked glucose polymer of at least 3 glucose residues wherein at least one hydroxyl group of the polymer is alkylated with a HBr salt of 1-amino-3-bromopropane resulting in a primary propyl amine.

52. The composition of claim 51 wherein the primary amine is reductively aminated with an aldehyde.

53. The composition of claim 52 wherein the aldehyde is a reducing sugar.

54. The composition of claim 53 wherein the reducing sugar is glucose, galactose, mannose, maltose, melbiose, cellobiose, maltopentaose, N-acetylglucosamine, or lactose.

55. The composition of claim 51 wherein the primary amine has been transformed into one of a substituted sulfonamide, amide, urea, or thiourea.

56. The composition of claim 55 wherein the substitution on the sulfonamide is methyl, 2-propyl, 1-propyl, isobutyl, cyclohexyl, benzyl, phenyl, or p-toluenyl.

57. The composition of claim 55 in which the amine is reacted with benzenedisulfonyl chloride resulting in a crosslinked glucan.

58. A composition comprising a chemically modified beta 1,3 linked glucose polymer of at least 3 glucose residues in which at least one primary hydroxyl group of the polymer is oxidized to a carboxylic acid.

59. The composition of claim 58 wherein the carboxylic acid is converted to a substituted amide by coupling with an amine.

60. The composition of claim 59 in which the amine comprises one of benzyl amine, 1,3-diaminopropane, serotonin, tryptamine, histamine, 4-aminomethylpyridine, R-(−)-2-phenyl glycinol L-(−)-2-amino-3-phenyl-1-propanol, furfuryl amine, or 1-(3-aminopropyl) imidazole.
